(19) United States
(12) Patent Application Publication
(10) Pub. No.: US 2015/0057243 A1
(43) Pub. Date: Feb. 26, 2015

(54) Compositions and Methods for the Inhibition of Methyltransferases

(71) Applicant: Northeastern University, Boston, MA (US)

(72) Inventors: Zhaohui Sunny Zhou, Wellesley, MA (US); Wanlu Qu, Yantai (CN); Tianyi Bai, Shanghai (CN)

(73) Assignee: Northern University, Boston, MA (US)

(21) Appl. No.: 14/389,984

(22) PCT Filed: Apr. 2, 2013
(86) PCT No.: PCT/US13/34913

§ 371 Date: Oct. 1, 2014

Related U.S. Application Data
(60) Provisional application No. 61/619,113, filed on Apr. 2, 2012.

(51) Int. Cl. C07H 19/16; C12N 9/10; C07C 323/58; C12Q 1/48; C07H 1/00; C12P 19/40

(52) U.S. Cl. CPC C07H 19/16; C07H 1/00; C12P 19/40; C07C 323/58; C12Q 1/48; C12N 9/1007
USPC 514/45; 536/27.6; 435/88; 562/556; 435/184; 435/15; 435/193

(57) ABSTRACT

Methods and compositions disclosed herein relate to detecting, analyzing, isolating and inhibiting methyltransferases, methyltransferase substrates, S-adenosyl-methionine-binding proteins and RNA, including for the treatment of disease.

### PRIORITY

This application claims priority to U.S. Provisional Patent Application No. 61/619,113, filed Apr. 2, 2012, the contents of which are hereby incorporated by reference in their entirety.

### TECHNICAL FIELD

The field of this application generally relates to the medical and detection fields. More specifically, the field relates to detecting, analyzing, isolating and inhibiting methyltransferases, methyltransferase substrates, S-adenosyl-methionine-binding proteins and RNA, including for the treatment of disease.

### BACKGROUND

Often dubbed as Nature's machinery, enzymes play central roles in biology and diseases. Naturally, it is critical to know what each machine builds or breaks; in other words, the substrates of each enzyme. This however remains a tall order. Fundamentally, the interaction between an enzyme and its substrates or products is transient, i.e. to carry its function, an enzyme does not form a “permanently-associated” complex with its substrates or products.

S-Adenosyl-methionine (AdoMet or SAM)-dependent methylations play diverse and pivotal roles in biology and diseases. Methylation is a common protein post-translational modification (PTM) and also a common modification of DNA, RNA, drugs, xenobiotics, metabolites, lipids and carbohydrates. From the perspective of enzymology, methylation is a group-transfer reaction that typically involves a methyltransferase (“MTase”), AdoMet as the methyl donor and a nucleophilic substrate (e.g., histone), typically via an S_{N}2 mechanism without intermediates. AdoMet serves as methyl donor for a large family of methyltransferases (MTases) and is also used in other enzymes (such as radical SAM family) and proteins (such as cystathionine beta-lyase and cystathionine beta-synthase) and AdoMet-binding RNAs (aka riboswitches, natural regulatory RNA aptamers that appear to sense small molecules). See, e.g., Epshtein, V. et al. Proc. Nat'l Acad. Sci. U.S.A. 2003, 100(9), 5056-5056.

However, methylation reactions are not particularly amenable to study and the reactants can be difficult to isolate. First, methylation products readily dissociate from the MTases, making it difficult to match the substrates with their corresponding MTases. Second, methylation poses significant challenges for its analysis. This can largely be attributed to the intrinsic chemical properties of the methyl group: it is small (15 Da), free of charge, and inert. For these reasons alone, commonly used approaches (e.g., mass spectrometry, immunoassays, selective tagging and affinity enrichment) have limited utility, particularly for global analysis and discovery proteomics.

Thus, there remains a need for a facile, efficient, and accurate method for detecting, isolating, and studying the methyltransferases that participate in the reactions and the substrates that these enzymes utilize, in addition to methods for inhibiting selected methyltransferases for various applications, including the treatment of diseases.

### SUMMARY

The present disclosure provides methods and compositions for the study of AdoMet-binding proteins, such as methyltransferases, and the reactants in reactions, including methylation reactions. The present disclosure takes advantage of methyltransferase's and other AdoMet-binding proteins' high binding affinity for AdoMet, typically binding in the low micromolar to submicromolar range. As such, the use of AdoMet analogs, as described herein, that can be covalently linked to a substrate allow for the isolation, detection, and study of methyltransferase reactions and the actors participating in the reaction. As shown in FIG. 14, an AdoMet analog can be bound by a methyltransferase (MTase). The MTase can also bind to a natural or artificial substrate or “hook” on an AdoMet analog and catalyze the methyl transfer reaction. However, rather than generating a labile methylation product, the substrate is covalently linked to the AdoMet analog. The resulting “bi-substrate adduct” (substrate-AdoMet analog) may bind to the enzyme with even higher affinity due to the additional favorable binding interaction between the MTase and its nucleophilic substrate.

For substrate identification, the bisubstrate adduct can be formed in situ, catalyzed by the MTase of interest via cross-linking of the substrate with a suitable AdoMet analog, as illustrated in FIG. 14. Again, synergetic interactions between the MTase and the resulting adduct will likely result in tight binding, allowing the substrate to be affinity-enriched. Even if the adduct dissociates from the enzyme, the substrate is covalently attached to an adenosyl group. Because most proteins do not contain adenosine, commercially available anti-adenosine antibodies can be used for detection and affinity enrichment to facilitate the subsequent analysis (e.g., mass spectrometry).

In other aspects, the present disclosure provides compositions and methods for the inhibition of methyltransferases using a “bi-substrate adduct.” In certain embodiments, the bisubstrate adduct is formed in situ, catalyzed by the MTase of interest via cross-linking of the substrate with a suitable AdoMet analog, as illustrated in FIG. 14. Synergetic interactions between the MTase and the resulting adduct result in tight binding, allowing the bisubstrate-adduct to compete with MTase substrates for binding and thereby inhibit the enzyme.

In other aspects, the present disclosure provides methods for modifying AdoMet-binding proteins using AdoVin and AdoVin analogs. Because the close structural similarity to AdoMet (the natural ligand), AdoVin and its analogs bind to AdoMet-binding proteins with similar binding affinity. Due to the higher intrinsic chemical reactivity of the vinyl sulfonium group in AdoVin and its analogs, nucleophilic groups at or near the AdoMet-binding sites of the proteins react with AdoVin and its analogs, resulting in protein modification.

The methods and compounds disclosed herein are also useful for inhibiting, isolating, purifying any enzyme that binds to AdoMet.

### BRIEF DESCRIPTION OF THE DRAWINGS

The following figures are provided for the purpose of illustration only and are not intended to be limiting.

FIG. 1 shows HPLC chromatograms of AdoVin synthesis reactions.

FIG. 2 shows an extracted ion chromatogram of an AdoVin synthesis reaction.

FIG. 3 shows a mass spectrum of an AdoVin synthesis reaction.

FIG. 4 shows an MS/MS spectrum of the precursor ion of AdoVin.

FIG. 5 shows an HPLC chromatogram of a reaction between AdoVin and TNB catalyzed by TPMT.

FIG. 6 shows HPLC chromatograms of a reaction between AdoVin and TNB with and without TPMT and without AdoMet synthetase.

FIG. 7 shows overlayed UV spectra of adenosine, methyl-TNB and an AdoVin-TNB adduct.

FIG. 8 shows a ¹H Nuclear Magnetic Resonance (NMR) spectrogram of vinthionine.

FIG. 9 shows the calculated mass for TNB, AdoVin and their adduct.

FIG. 10 shows an extracted ion chromatogram of reactions between AdoVin and TNB with or without TPMT.

FIG. 11 shows a mass spectrum of a reaction between AdoVin and TNB.

FIG. 12 shows an MS/MS spectrum of the precursor ion of an AdoVin-TNB adduct.

FIG. 13 shows a scheme demonstrating the reactions between methyltransferase and AdoMet and an analog or derivative of AdoMet.

FIG. 14 shows the formation of a bisubstrate adduct for both AdoMet and an AdoMet analog or derivative.

FIG. 15 shows reactions between AdoMet and an AdoMet aziridinium analog or derivative and methyltransferase.

FIG. 16 shows a reaction between methyltransferase and AdoVin to form a bisubstrate adduct.

FIG. 17 shows a synthesis of AdoMet from methionine and ATP and AdoVin from vinthionine and ATP catalyzed by AdoMet synthetase.

FIG. 18 shows a synthesis of Ado-SeVin from selenovinthionine and ATP catalyzed by AdoMet synthetase.

FIG. 19 shows a generic scheme depicting synthesis of AdoVin analogs or derivatives.

FIG. 20 shows a generic scheme depicting synthesis of AdoVin analogs or derivatives.

FIG. 21 shows a generic scheme depicting synthesis of AdoVin analogs or derivatives.

FIG. 22 shows a generic scheme depicting synthesis of AdoVin analogs or derivatives.

FIG. 23 shows a generic scheme depicting synthesis of AdoVin analogs or derivatives.

FIG. 24 shows illustrations of transmethylation reactions of AdoMet and AdoVin and its analogs.

FIG. 25A illustrates the mechanism for the transfer of a methyl group from AdoMet to DNA substrate catalyzed by DNA methyltransferase and the role of the catalytic cysteine in the active site. FIG. 25B illustrates the mechanism by which AdoVin forms a bisubstrate-adduct with the DNA substrate.

FIG. 26 shows a UV/Vis absorbance spectrum overlay of pure AdoMet and synthesized AdoVin.

FIG. 27 shows a strong cation exchange (SCX) HPLC chromatogram of AdoVin and AdoMet synthesis reactions.

FIG. 28 shows absorbance changes associated with formation of an AdoVin-TNB adduct.

FIG. 29 shows a bisubstrate-adduct and TPMT complex binding assay.

FIG. 30 shows an extracted ion chromatogram (XIC) of AdoVin-TNB binding assays.

FIG. 31 HPLC chromatograms of reactions between AdoVin and 4-nitrobenzenethiol (4-NBT).

FIG. 32 shows HPLC chromatograms of reactions between AdoVin and 4-nitrobenzenethiol (4-NBT) in the presence and absence of TPMT and MAT.

FIG. 33 shows UV spectra of 4-NBT and the AcoVin-4-NBT adduct.

FIG. 34 shows the time-dependent UV absorbance change of 4-methoxybenzylthiol and 4-nitrophenyl in a specificity assay with AdoVin.

FIG. 35 shows a ¹³C Nuclear Magnetic Resonance (NMR) spectrogram of vinthionine.

FIG. 36 shows a selected ion monitoring (XIC) chromatogram of 546 m/x (the expected AdoVin-Hcy adduct at 1.47 min.

FIG. 37A illustrates the transfer of a methyl group from AdoMet to DNA catalyzed by DNA methyltransferase. FIG. 37B illustrates the direct covalent modification of the catalytic cysteine in the active site by AdoVin.

FIG. 38 shows a tandem mass spectrum of an AdoVin-Hcy adduct.

FIG. 39 shows a tandem mass spectrum of an AdoVin-Hcy adduct in a negative control reaction.

### DETAILED DESCRIPTION

The patent and scientific literature referred to herein establishes knowledge that is available to those of skill in the art. The issued U.S. patents, allowed applications, published foreign applications, and references, that are cited herein are hereby incorporated by reference to the same extent as if each was specifically and individually indicated to be incorporated by reference.

For convenience, certain terms employed in the specification, examples and claims are collected here. Unless defined otherwise, all technical and scientific terms used in this disclosure have the same meanings as commonly understood by one of ordinary skill in the art to which this disclosure belongs. The initial definition provided for a group or term provided in this disclosure applies to that group or term throughout the present disclosure individually or as part of another group, unless otherwise indicated.

The compounds of this disclosure include any and all possible isomers, stereoisomers, enantiomers, diastereomers, tautomers, pharmaceutically acceptable salts, hydrates and solvates thereof, as well as crystalline polymorphic forms of the disclosed compounds and any and all possible isomers, stereoisomers, enantiomers, diastereomers, tautomers, pharmaceutically acceptable salts, hydrates and solvates thereof. Thus, the terms “compound” and “compounds” as used in this disclosure refer to the compounds of this disclosure and any and all possible isomers, stereoisomers, enantiomers, diastereomers, tautomers, pharmaceutically acceptable salts, solvates, hydrates and crystalline polymorphs thereof.

### DEFINITIONS

The term “alkyl” as used herein refers to a straight or branched carbon chain, wherein alkyl chain length is indicated by a range of numbers. In exemplary embodiments, “alkyl” refers to an alkyl chain as defined above containing 1, 2, 3, 4, 5, 6, 7, 8, 9 or 10 carbons (i.e. C₁₋₁₀alkyl). Examples of an alkyl group include, but are not limited to, methyl, ethyl, propyl, iso-propyl, butyl, iso-butyl, secondary-butyl, tertiary-butyl, pentyl, iso-pentyl, neo-pentyl, hexyl, iso-hexyl, 3-methylpentyl, 2,3-dimethylbutyl and neo-hexyl.

The term “alkenyl” as used herein refers to a straight or branched carbon chain containing at least one carbon-carbon double bond. In exemplary embodiments, “alkenyl” refers to a carbon chain as defined above containing 2, 3, 4, 5, 6, 7, 8, 9 or 10 carbons (i.e. C₂₋₁₀alkenyl). Examples of an alkenyl group include, but are not limited to, ethene, propene, butene, pentene, hexene, heptene, octene, nonene and decene.

The term “alkynyl” as used herein refers to a straight or branched carbon chain containing at least one carbon-carbon triple bond. In exemplary embodiments, “alkynyl” refers to a carbon chain as defined above containing 2, 3, 4, 5, 6, 7, 8, 9 or 10 carbons (i.e. C₂₋₁₀alkynyl). Examples of an alkynyl group include, but are not limited to, ethyne, propyne, butyne, pentyne, hexyne, heptyne, octyne, nonyne and decyne.

The term “aryl” as used herein refers to a cyclic hydrocarbon, where the ring is characterized by delocalized π electrons (aromaticity) shared among the ring members, and wherein the number of ring atoms is indicated by a range of numbers. In exemplary embodiments, “aryl” refers to a cyclic hydrocarbon as described above containing 6, 7, 8, 9, or 10 ring atoms (i.e. C₆₋₁₀aryl). Examples of an aryl group include, but are not limited to, benzene, naphthalene, tetralin, indene, and indane.

The term “cycloalkyl” as used herein refers to a monocyclic saturated carbon ring, wherein the number of ring atoms is indicated by a range of numbers. In exemplary embodiments, “cycloalkyl” refers to a carbon ring as defined above containing 3, 4, 5, 6, 7, or 8 ring atoms (i.e. C₃₋₈cycloalkyl). Examples of a cycloalkyl group include, but are not limited to, cyclopropyl, cyclobutyl, cyclopentyl, cyclohexyl, cycloheptyl, and cyclooctyl.

The term “heterocycle” or “heterocyclyl” as used herein refers to a cyclic hydrocarbon, wherein at least one of the ring atoms is an O, N, S, P or Se, wherein the number of ring atoms is indicated by a range of numbers. Heterocyclyl moieties as defined herein have C, N, S, P or Se bonding hands. For example, in some embodiments, a ring N atom from the heterocyclyl is the bonding atom to —C(O) to form an amide, carbamate, or urea. In exemplary embodiments, “heterocyclyl” refers to a cyclic hydrocarbon as described above containing 4, 5, or 6 ring atoms (i.e. C₄₋₆heterocyclyl). Examples of a heterocycle group include, but are not limited to, aziridine, oxirane, thiirane, azetidine, oxetane, thietane, pyrrolidine, tetrahydrofuran, pyran, thiopyran, thiomorpholine, thiomorpholine S-oxide, thiomorpholine S-dioxide, oxazoline, tetrahydrothiophene, piperidine, tetrahydropyran, thiane, imidazolidine, oxazolidine, thiazolidine, dioxolane, dithiolane, piperazine, oxazine, dithiane, and dioxane.

The term “heteroaryl” as used herein refers to a cyclic hydrocarbon, where at least one of the ring atoms is an O, N, S, P or Se, the ring is characterized by delocalized π electrons (aromaticity) shared among the ring members, and wherein the number of ring atoms is indicated by a range of numbers. Heteroaryl moieties as defined herein have C, N, S, P or Se bonding hands. For example, in some embodiments, a ring N atom from the heteroaryl is the bonding atom to —C(O) to form an amide, carbamate, or urea. In exemplary embodiments, “heteroaryl” refers to a cyclic hydrocarbon as described above containing 5 or 6 ring atoms (i.e. C₅₋₆heteroaryl). Examples of a heteroaryl group include, but are not limited to, pyrrole, furan, thiene, oxazole, thiazole, isoxazole, isothiazole, imidazole, pyrazole, oxadiazole, thiadiazole, triazole, tetrazole, pyridine, pyrimidine, pyrazine, pyridazine, and triazine.

The term “ketone” as used herein refers to a moiety containing at least one carbonyl group where the carbonyl carbon is bound to two other carbon atoms. In exemplary embodiments, “ketone” refers to a carbonyl-containing moiety as described above containing 3, 4, 5, 6, 7, 8, 9 or 10 carbon atoms (i.e. C₃₋₁₀ketone). Examples of a ketone group include, but are not limited to, acetone, butanone, pentanone, hexanone, heptanone, octanone, nonanone, decanone, cyclobutanone, cyclopentanone, cyclohexanone, cycloheptanone, cyclooctanone, cyclononanone and cyclodecanone.

The term “aldehyde” as used herein refers to a moiety containing at least one carbonyl group where the carbonyl carbon is bound to a carbon atom and a hydrogen atom. In exemplary embodiments, “aldehyde” refers to a carbonyl-containing moiety as described above containing 2, 3, 4, 5, 6, 7, 8, 9 or 10 carbon atoms (i.e. C₂₋₁₀aldehyde). Examples of an aldehyde group include, but are not limited to, formaldehyde, acetaldehyde, propionaldehyde, butyraldehyde, pentanal, hexanal, heptanal, octanal, nonanal, decanal, cyclopropanecarbaldehyde, cyclobutanecarbaldehyde, cyclopentanecarbaldehyde, cyclohexanecarbaldehyde, cycloheptanecarbaldehyde, cyclooctanecarbaldehyde and cyclononanecarbaldehyde.

The term “carboxylic acid” as used herein refers to a group containing a carbonyl group where the carbonyl carbon is bound to an oxygen atom bearing either a hydrogen atom or a negative charge (i.e. an “acid” or “carboxylate” group). In exemplary embodiments, “carboxylic acid” refers to an acid or carboxylate-containing moiety as described above containing 1, 2, 3, 4, 5, 6, 7, 8, 9, 10, 11 or 12 carbon atoms (i.e. C₁₋₁₂carboxylic acid). Examples of carboxylic acids include, but are not limited to formic acid, acetic acid, propionic acid, butanoic acid, pentanoic acid, hexanoic acid, heptanoic acid, octanoic acid, nonanoic acid, decanoic acid, cyclopropanecarboxylic acid, cyclobutanecarboxylic acid, cyclopentanecarboxylic acid, cyclohexanecarboxylic acid, cycloheptanecarboxylic acid, cyclooctanecarboxylic acid, or cyclononanecarboxylic acid.

The term “amino alcohol” as used herein refers to a functional group containing both an alcohol and an amine group. As used herein, “amino alcohols” also refers to amino acids as defined above having a carbon bound to an alcohol in place of the carboxylic acid group. In exemplary embodiments, the term “amino alcohol” refers to an amino alcohol as defined above wherein the amine is bound to the carbon adjacent to the alcohol-bearing carbon. In exemplary embodiments, “amino alcohol” refers to an amine and alcohol-containing moiety as described above containing 1, 2, 3, 4, 5, 6, 7, 8, 9, 10, 11 or 12 carbon atoms (i.e. C₁₋₁₂amino alcohol). Examples of amino alcohols include, but are not limited to, ethanolamine, heptaminol, isoetarine, norepinephrine, propanolamine, sphingosine, methanolamine, 2-amino-4-mercaptobutan-1-ol, 2-amino-4-(methylthio)butan-1-ol, cysteinol, phenylglycinol, prolinol, 2-amino-3-phenyl-1-propanol, 2-amino-1-propanol, cyclohexylglycinol, 4-hydroxy-prolinol, leucinol, tert-leucinol, phenylalaminol, α-phenylglycinol, 2-pyrrolidinemethanol, tyrosinol, valinol, serinol, 2-dimethylaminoethanol, histidinol, isoleucinol, leucinol, methioninol, 1-methyl-2-pyrrolidinemethanol, threoninol, tryptophanol, alaminol, argininol, glycinol, glutaminol, 4-amino-5-hydroxypentanamide, 4-amino-5-hydroxypentanoic acid, 3-amino-4-hydroxybutanoic acid, lysinol, 3-amino-4-hydroxybutanamide, and 4-hydroxy-prolinol.

The term “nucleoside” as used herein refers to natural and synthetic glycosylamines comprising a nucleobase bound to a ribose, pentose, hexose, open-chain, deoxyribose, deoxypentose, deoxyhexose, or deoxy-open-chain sugar via a beta-glycosidic linkage. Examples of a nucleoside include, but are not limited to, adenosine, deoxyadenosine, guanosine, deoxyguanosine, 5-methyluridine, thymidine, uridine, deoxyuridine, cytidine, deoxycytidine, formycin, aristeromycin, didanosine, inosine, acyclovir, and deoxyinosine.

The term “nucleotide” as used herein refers to a nucleoside molecule as defined above bonded to one or more phosphate groups.

The term “nucleoside derivative” as used herein refers to natural and synthetic nucleoside analogs which are modified at at least one position, either by addition of a functional group or atom, removal of a functional group or atom or change of a functional group or atom to a different functional group or atom (including, but not limited to, isotopes). Examples of nucleoside derivatives include, but are not limited to, abacavir, N4-acetylcytidine, allopurinol riboside, 2′-O-allyladenosine, 3′-O-allyladenosine, 3′-O-allylcytidine, 2′-O-allylcytidine, 2′-O-allylguanosine, 3′-O-allylguanosine, 2′-O-allyluridine, 3′-O-allyluridine, bromodeoxyuridine, cytarabine, azacitidine, decitabine, pseudouridine, S-adenosyl-L-homocysteine, pentostatin, regadenoson, telbivudine, 8-oxo-2′-deoxyguanosine, CGS-21680, floxuridine, 5-methyluridine, dihydrouridine, nelarabine, xanthosine, maribavir, 8-hydroxyguanosine, N4-chloroacetylcytosine arabinoside, sapacitabine, orotidine, queuosine, lysidine, fialuridine, CP-532,903, cordycepin, tezacitabine, dexelvucitabine, N6-cyclopentyladenosine, iododeoxyuridine, PSI-6130, 5,6-dichloro-1-beta-D-ribofuranosylbenzimidazole, S-adenosylmethioninamine, FV-100 and 5-ethynyl-2′-deoxyuridine, 9-β-D-allopyranosyl-9H-Purin-6-amine, (S)-9-(2,3-dihydroxypropyl)adenine (DHPA), D-eritadenine, 9-(2-bromo-4-hydroxy-3-hydroxymethyl-2-butenyl)adenine, 1-(6-amino-9H-purin-9-yl)-1,5-dideoxy-D-Arabinitol, S-8-aza-adenosylmethionine (8-aza-SAM), S-2-aminopurinylmethionine (2AP-SAM), S-2,6-diaminopurinylmethionine (DAPSAM), and 2,6-diaminopurine (DAP).

The term “nucleotide derivative” as used herein refers to a nucleoside derivative molecule as defined above bonded to one or more phosphate groups.

The term “amino acid” as used herein refers to a group containing a carboxylic acid and an amine bound to the carbon atom immediately adjacent to the carboxylate group, and includes both natural and synthetic amino acids. Examples of amino acids include, but are not limited to, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan

The terms “analog” and “derivative” as used herein are interchangeable and refer to a natural or non-natural modification of at least one position of a given molecule. For example, a derivative of a given compound or molecule is modified either by addition of a functional group or atom, removal of a functional group or atom or change of a functional group or atom to a different functional group or atom (including, but not limited to, isotopes)

The term “electrophile” as used herein refers to a functional group which can participate in a chemical reaction by accepting an electron pair in order to bond to a nucleophile. Examples of electrophiles include, but are not limited to, alkenes, alkynes, epoxides, aziridines, oxiranes, azetidines, aldehydes, ketones, esters, carboxylic acids, carboxylates, imines, imides, azides, azo groups, eneamines, alkyl halides, alkenyl halides, alkynyl halides, aryl halides, phosphines, phosphine oxides, phosphinites, phosphonites, phosphites, phohsphonates, phosphates, sulfates, sulfoxides, sulfonyl groups, sulfoxyl groups, sulfonates, nitrates, nitrites, nitriles, nitro groups, nitroso groups, cyanates, thiocyanates, isothiocyanates, carbonates, acyl halides, peroxides, hydroperoxides, hemiacetals, hemiketals, acetals, ketals, orthoesters, orthocarbonate esters, sulfides, disulfides, sulfonic acids, sulfonic acids, thiones, thials, phosphodiesters, vinyl sulfoxides, vinyl sulfones, alpha-beta unsaturated carbonyl compounds (including, but not limited to ketones, aldehydes, acids, esters and amides), boronic acids, boronic esters, boronic acids and boronic esters.

The term “heteroatom” as used herein refers to any atom that is not carbon or hydrogen. Examples of heteroatoms include, but are not limited to, He, Li, Be, B, N, O, F, Ne, Na, Mg, Al, Si, P, S, Cl, Ar, K, Ca, Sc, Ti, V, Cr, Mn, Fe, Co, Ni, Cu, Zn, Ga, Ge, As, Se, Br, Kr, Rb, Sr, Y, Zr, Nb, Mo, Tc, Ru, Rh, Pd, Ag, Cd, In, Sn, Sb, Te, I, Xe, Cs, Ba, Lu, Hf, Ta, W, Re, Os, Ir, Pt, Au, Hg, Tl, Pb, Bi, Po, At, Rn, Fr, Ra, Lr, Rf, Db, Sg, Bh, Hs, Mt, Ds, Rg, Cn, Uut, Fl, Uup, Lv, Uus, Uuo, La, Ce, Pr, Nd, Pm, Sm, Eu, Gd, Tb, Dy, Ho, Er, Tm, Yb, Ac, Th, Pa, U, Np, Pu, Am, Cm, Bk, Cf, Es, Fm, Md and No.

In the compounds described herein, heteroatoms are capable of bearing multiple different valencies. By way of non-limiting example, S, Se and N can be neutral or hold a positive charge, and O can be neutral or hold a positive or negative charge.

In the structures described herein, a dashed line indicates that the bond can be a single, double or triple bond.

The term “substituted” in connection with a moiety as used herein refers to a further substituent which is attached to the moiety at any acceptable location on the moiety. Unless otherwise indicated, moieties can bond through a carbon, nitrogen, oxygen, sulfur, or any other acceptable atom. Examples of substituents include, but are not limited to amines, alcohols, thiols, ethers, alkenes, alkynes, epoxides, aziridines, oxiranes, azetidines, dihydrofurans, pyrrolidines, pyrans, piperidines, aldehydes, ketones, esters, carboxylic acids, carboxylates, imines, imides, azides, azo groups, eneamines, alkyl halides, alkenyl halides, alkynyl halides, aryl halides, phosphines, phosphine oxides, phosphinites, phosphonites, phosphites, phohsphonates, phosphates, sulfates, sulfoxides, sulfonyl groups, sulfoxyl groups, sulfonates, nitrates, nitrites, nitriles, nitro groups, nitroso groups, cyanates, thiocyanates, isothiocyanates, carbonates, acyl halides, peroxides, hydroperoxides, hemiacetals, hemiketals, acetals, ketals, orthoesters, orthocarbonate esters, sulfides, disulfides, sulfonic acids, sulfonic acids, thiones, thials, phosphodiesters, boronic acids, boronic esters, boronic acids and boronic esters.

The term “halogen” as used herein refers to a fluorine, chlorine, bromine or iodine atom.

The term “halide” as used herein refers to a functional group containing an atom bond to a fluorine, chlorine, bromine or iodine atom. Exemplary embodiments disclosed herein may include “alkyl halide,” “alkenyl halide,” “alkynyl halide,” “cycloalkyl halide,” “heterocyclyl halide,” or “heteroaryl halide” groups. In exemplary embodiments, “alkyl halide” refers to a moiety containing a carbon-halogen bond containing 1, 2, 3, 4, 5, 6, 7, 8, 9 or 10 carbon atoms (i.e. C₁₋₁₀alkyl halide). Examples of an alkyl halide group include, but are not limited to, fluoromethyl, fluoroethyl, chloromethyl, chloroethyl, bromomethyl, bromoethyl, iodomethyl and iodoethyl groups. Unless otherwise indicated, any carbon-containing group referred to herein can contain one or more carbon-halogen bonds. By way of non-limiting example, a C₁alkyl group can be, but is not limited to, methyl, fluoromethyl, difluoromethyl, trifluoromethyl, chloromethyl, dichloromethyl, trichloromethyl, bromomethyl, dibromomethyl, tribromomethyl, iodomethyl, diiodomethyl, triiodomethyl, chlorofluoromethyl, dichlorofluoromethyl, and difluorochloromethyl.

The term “salts” as used herein embraces pharmaceutically acceptable salts commonly used to form alkali metal salts of free acids and to form addition salts of free bases. The nature of the salt is not critical, provided that it is pharmaceutically acceptable. Suitable pharmaceutically acceptable acid addition salts may be prepared from an inorganic acid or from an organic acid. Exemplary pharmaceutical salts are disclosed in Stahl, P. H., Wermuth, C. G., Eds. Handbook of Pharmaceutical Salts: Properties, Selection and Use; Verlag Helvetica Chimica Acta/Wiley-VCH: Zurich, 2002, the contents of which are hereby incorporated by reference in their entirety. Specific non-limiting examples of inorganic acids are hydrochloric, hydrobromic, hydroiodic, nitric, carbonic, sulfuric and phosphoric acid. Appropriate organic acids include, without limitation, aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric or galacturonic acid. Suitable pharmaceutically acceptable salts of free acid-containing compounds disclosed herein include, without limitation, metallic salts and organic salts. Exemplary metallic salts include, but are not limited to, appropriate alkali metal (group Ia) salts, alkaline earth metal (group IIa) salts, and other physiological acceptable metals. Such salts can be made from aluminum, calcium, lithium, magnesium, potassium, sodium and zinc. Exemplary organic salts can be made from primary amines, secondary amines, tertiary amines and quaternary ammonium salts, for example, tromethamine, diethylamine, tetra-N-methylammonium, N,N′-dibenzylethylenediamine, chloroprocaine, choline, diethanolamine, ethylenediamine, meglumine (N-methylglucamine) and procaine.

The term “hydrate” as used herein refers to a compound disclosed herein which is associated with water in the molecular form, i.e., in which the H—OH bond is not split, and may be represented, for example, by the formula R.H₂O, where R is a compound disclosed herein. A given compound may form more than one hydrate including, for example, monohydrates (R.H₂O), dihydrates (R.2H₂O), trihydrates (R.3H₂O), and the like.

The term “solvate” as used herein refers to a compound disclosed herein which is associated with solvent in the molecular form, i.e., in which the solvent is coordinatively bound, and may be represented, for example, by the formula R.(solvent), where R is a compound disclosed herein. A given compound may form more than one solvate including, for example, monosolvates (R.(solvent)) or polysolvates (R.n(solvent)) wherein n is an integer greater than 1) including, for example, disolvates (R.2(solvent)), trisolvates (R.3(solvent)), and the like, or hemisolvates, such as, for example, R.n/2(solvent), R.n/3(solvent), R.n/4(solvent) and the like, wherein n is an integer. Solvents herein include mixed solvents, for example, methanol/water, and as such, the solvates may incorporate one or more solvents within the solvate.

Enantiomers are defined as one of a pair of molecular entities which are mirror images of each other and non-superimposable.

Diastereomers or diastereoisomers are defined as stereoisomers other than enantiomers. Diastereomers or diastereoisomers are stereoisomers not related as mirror images. Diastereoisomers are characterized by differences in physical properties, and by some differences in chemical behavior towards achiral as well as chiral reagents.

The term “tautomer” as used herein refers to compounds produced by the phenomenon wherein a proton of one atom of a molecule shifts to another atom. See March, Advanced Organic Chemistry: Reactions, Mechanisms and Structures, 4th Ed., John Wiley & Sons, pp. 69-74 (1992). Tautomerism is defined as isomerism of the general form

G-X—Y═Z⇄X═Y—Z-G

where the isomers (called tautomers) are readily interconvertible; the atoms connecting the groups X, Y and Z are typically any of C, H, O, or S, and G is a group which becomes an electrofuge or nucleofuge during isomerization. The most common case, when the electrofuge is H′, is also known as “prototropy.” Tautomers are defined as isomers that arise from tautomerism, independent of whether the isomers are isolable.

The following abbreviations are used in this disclosure and have the following definitions: ATP is adenosine triphosphate, Hcy is homocysteine, HPLC is high performance (or pressure) liquid chromatography, LC is liquid chromatography, MAT is methionine adenosyltransferase or AdoMet synthetase, MS is mass spectrometry, TFA is trifluoroacetic acid, TNB is 5-thio-2-nitrobenzoic Acid, TPMT is thiopurine methyltransferase, and UV is ultra-violet.

### 1. General

Disclosed herein are compositions for the detection of methyltransferases and methyltransferase substrates. Exemplary MTases include DNA methyltransferases, thiopurine methyltransferase, homocysteine methyltransferase and protein cysteine methyltransferase. Furthermore, such compositions are useful for the isolation, and purification of methyltransferases and their substrates. In particular embodiments, the compositions are used to inhibit specific methyltransferases in a sample or in a subject.

In addition, methods of detecting methyltransferases and their substrates, as well as methods of inhibiting a methyltransferase or a protein, are disclosed. In some embodiments, the protein binds to AdoMet and its analogs, AdoMet-dependent methyltransferases, polyamine synthetase and radical SAM enzymes. Furthermore, methods of purifying and isolating methyltransferases and their substrates are disclosed herein. Such methods utilize the compositions disclosed herein and allow for facile analysis of methyltransferases and their substrates. Additionally, the methods disclosed herein provide analytical tools useful in the detection of methyltransferases and their substrates in tissues and cells.

### 2. Compositions

The compositions disclosed herein comprise a compound of Formula I:

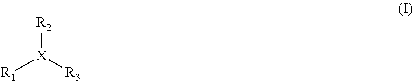

or a salt, hydrate or solvate thereof, wherein:
X is C, CR, N, NR, NOR, P, PR, POR, P(R)R′, P(OR)R′, P(OR)OR′, P(O), S, SR, SOR, S(R)R′, S(OR)R′, S(OR)OR′, S(O), S(R)(R′)R″, S(R)(R′)OR″, S(R)(OR′)OR″, S(OR)(OR′)OR″, S(O)R, S(O)OR, Se, SeR, SeOR, Se(R)R′, Se(OR)R′, Se(OR)OR′, Se(O), Se(R)(R′)R″, Se(R)(R′)OR″, Se(R)(OR′)OR″, Se(OR)(OR′)OR″, Se(O)R, Se(O)OR, SS, SeSe, SSe or SeS; and
R₁ is —R, a substituted or unsubstituted amino acid, C₁₋₁₂amino alcohol, C₁₋₁₂carboxylic acid, —OR, ═O, or R₁ and X taken together are:

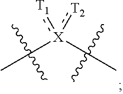

where T₁ and T₂ are each independently —OR or ═O;
R₂ is an electrophile;
X and R₂ taken together can form a 3-to-10-membered ring;
R₃ is a nucleotide, nucleoside or a derivative thereof; and
R, R′ and R″ are each independently H or a substituted or unsubstituted C₁₋₁₀alkyl, C₂₋₁₀alkenyl, C₂₋₁₀alkynyl, C₆₋₁₀aryl, C₃₋₈cycloalkyl, C₄₋₆heterocyclyl or C₅₋₆heteroaryl.

In certain embodiments disclosed herein, R₃ is one of adenosine, deoxyadenosine, guanosine, deoxyguanosine, 5-methyluridine, thymidine, uridine, deoxyuridine, cytidine, deoxycytidine, formycin, aristeromycin, didanosine, inosine, acyclovir, deoxyinosine, abacavir, N4-acetylcytidine, allopurinol riboside, 2′-O-allyladenosine, 3′-O-allyladenosine, 3′-O-allylcytidine, 2′-O-allylcytidine, 2′-O-allylguanosine, 3′-O-allylguanosine, 2′-O-allyluridine, 3′-O-allyluridine, bromodeoxyuridine, cytarabine, azacitidine, decitabine, pseudouridine, S-adenosyl-L-homocysteine, pentostatin, regadenoson, telbivudine, 8-oxo-2′-deoxyguanosine, CGS-21680, floxuridine, 5-methyluridine, dihydrouridine, nelarabine, xanthosine, maribavir, 8-hydroxyguanosine, N4-chloroacetylcytosine arabinoside, sapacitabine, orotidine, queuosine, lysidine, fialuridine, CP-532,903, cordycepin, tezacitabine, dexelvucitabine, N6-cyclopentyladenosine, iododeoxyuridine, PSI-6130, 5,6-dichloro-1-beta-D-ribofuranosylbenzimidazole, S-adenosylmethioninamine, FV-100 and 5-ethynyl-2′-deoxyuridine, 9-β-D-allopyranosyl-9H-Purin-6-amine, (S)-9-(2,3-dihydroxypropyl)adenine (DHPA), D-eritadenine, 9-(2-bromo-4-hydroxy-3-hydroxymethyl-2-butenyl)adenine, 1-(6-amino-9H-purin-9-yl)-1,5-dideoxy-D-Arabinitol, S-8-aza-adenosylmethionine (8-aza-SAM), S-2-aminopurinylmethionine (2AP-SAM), S-2,6-diaminopurinylmethionine (DAPSAM), and 2,6-diaminopurine (DAP). In addition, the electrophile can be a substituted or unsubstituted C₂₋₁₀alkene, C₂₋₁₀alkyne, C₂₋₁₀ketone, C₁₋₁₀aldehyde or C₁₋₁₀alkyl halide. Furthermore, the nucleotide, nucleoside or derivative thereof is bound through the pentose ring, hexose ring, or through the open-chain.

In certain embodiments disclosed herein, the electrophile is a substituted or unsubstituted C₂₋₁₀alkene, C₂₋₁₀alkyne, C₂₋₁₀ketone, C₁₋₁₀aldehyde or C₁₋₁₀alkyl halide.

In certain embodiments disclosed herein, the nucleotide, nucleoside or derivative thereof is bound through the pentose ring, hexose ring, or through the open-chain.

In some embodiments, the nucleotide, nucleoside or derivative thereof is bound through the 5′ position of the pentose ring. In other embodiments, the compound is a compound of Formula II or Formula III:

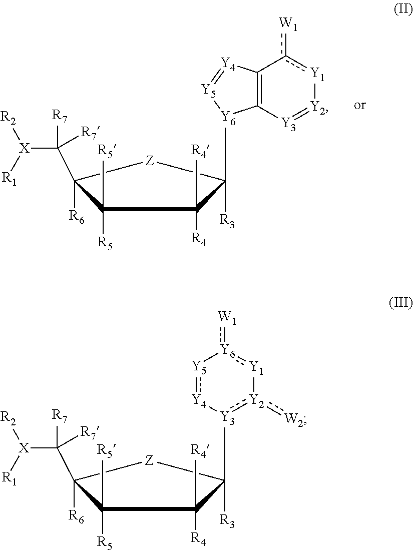

wherein W₁ and W₂ are each independently selected from R, O, OR, OC(O)R, OC(O)OR, OC(O)N(R)R′, N(R)R′, NC(O)R, NC(O)OR, NC(O)N(R)R′, P, PR, POR, P(R)R′, P(OR)R′, P(OR)OR′, P(O), P(R)(R′)R″, P(R)(R′)OR″, P(R)(OR′)OR″, P(OR)(OR′)OR″, P(O)R, P(O)OR, P(R)(R′)(R″)R′, P(R)(R′)(R″)OR″, P(R)(R′)(OR″)OR′″, P(R)(OR′)(OR″)OR′″, P(OR)(OR′)(OR″)OR′, P(O)(R)R′, P(O)(R)OR′, P(O)(OR)OR″, PO₂, S, SR, SOR, S(R)R′, S(OR)R′, S(OR)OR′, S(O), S(R)(R′)R″, S(R)(R′)OR″, S(R)(OR′)OR″, S(OR)(OR′)OR″, S(O)R, S(O)OR, S(R)(R′)(R″)R′″, S(R)(R′)(R″)OR′″, S(R)(R′)(OR″)OR′″, S(R)(OR′)(OR″)OR′, S(OR)(OR′)(OR″)OR′″, S(O)(R)R′, S(O)(R)OR′, S(O)(OR)OR″, SO₂, S(R)(R′)(R″)(R′″)R″″, S(R)(R′)(R″)(R′″)OR″″, S(R)(R′)(R″)(OR′″)OR″″, S(R)(R′)(OR″)(OR′″)OR″, S(R)(OR′)(OR″)(OR′″)OR″″, S(OR)(OR′)(OR″)(OR′″)OR″″, S(O)(R)(R′)R″, S(O)(R)(R′)OR″, S(O)(R)(OR′)OR″, S(O)(OR)(OR′)OR″, SO₂R, SO₂OR, Se, SeR, SeOR, Se(R)R′, Se(OR)R′, Se(OR)OR′, Se(O), Se(R)(R′)R″, Se(R)(R′)OR″, Se(R)(OR′)OR″, Se(OR)(OR′)OR″, Se(O)R, Se(O)R, Se(R)(R′)(R″)R′″, Se(R)(R′)(R″)OR′″, Se(R)(R′)(OR″)OR′″, Se(R)(OR′)(OR″)OR′″, Se(OR)(OR′)(OR″)OR′, Se(RO)(R)R′, Se(RO)(R)OR′, Se(O)(OR)OR″, SeO₂, Se(R)(R′)(R″)(R′″)R″″, Se(R)(R′)(R″)(R′″)OR″″, Se(R)(R′)(R″)(OR′″)OR″, Se(R)(R′)(OR″)(OR′″)OR″, Se(R)(OR′)(OR″)(OR′″)OR″″, Se(OR)(OR′)(OR″)(OR′″)OR″″, Se(RO)(R)(R′)R″, Se(RO)(R)(R′)OR″, Se(RO)(R)(OR′)OR″, Se(O)(OR)(OR′)OR″, SeO₂R, SeO₂OR, SSR, SeSeR, SSeR, or SeSR;

Y₁, Y₂, Y₃, Y₄, Y₅ and Y₆ are each independently selected from C, CR, CC(O)R, CC(O)OR, CC(O)N(R)R′, CN(R)R′, N, NR, NC(O)R, or NC(O)OR and Z is R, O, N(R)R′, S, S(O), or SO₂;

R₃, R₄, R_{4′}, R₅, R_{5′}, R₆, R₇ and R_{7′} are each independently selected from —R, —OR, —N(R)R′, —C(O)R, —C(O)OR, —C(O)N(R)R′, a substituted or unsubstituted amino acid, C₁₋₁₂amino alcohol, or C₁₋₁₂carboxylic acid; and

R, R′ R″, R′ and R″″ are each independently H or a substituted or unsubstituted C₁₋₁₀alkyl, C₂₋₁₀alkenyl, C₂₋₁₀alkynyl, C₆₋₁₀aryl, C₃₋₈cycloalkyl, C₄₋₆heterocyclyl or C₅₋₆heteroaryl.

Furthermore, the disclosed compounds can also have R₂ being selected from —C(R)═C(R)R′, —C□CR, or wherein R₂ and X taken together form:

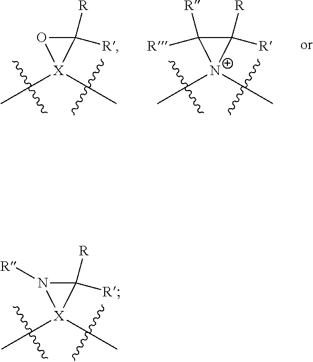

wherein R, R′ R″ and R′ are each independently H or a substituted or unsubstituted C₁₋₁₀alkyl, C₂₋₁₀alkenyl, C₂₋₁₀alkynyl, C₆₋₁₀aryl, C₃₋₈cycloalkyl, C₄₋₆heterocyclyl or C₅₋₆heteroaryl.

In some embodiments, the compound is a compound of Formula V, Formula VI or Formula VII:

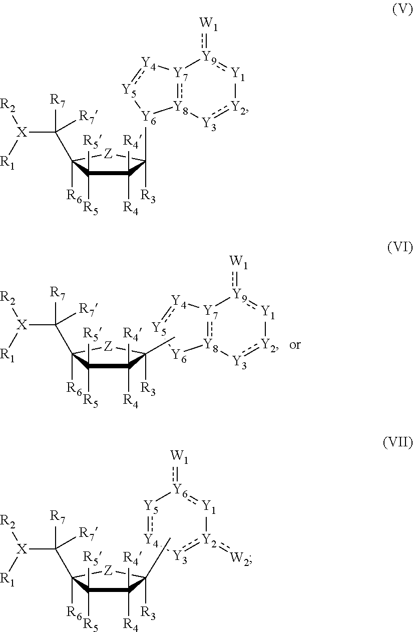

wherein W₁ and W₂ are each independently selected from R, O, OR, OC(O)R, OC(O)OR, OC(O)N(R)R′, N(R)R′, NC(O)R, NC(O)OR, NC(O)N(R)R′, P, PR, POR, P(R)R′, P(OR)R′, P(OR)OR′, P(O), P(R)(R′)R″, P(R)(R′)OR″, P(R)(OR′)OR″, P(OR)(OR′)OR″, P(O)R, P(O)OR, P(R)(R′)(R″)R′, P(R)(R′)(R″)OR′″, P(R)(R′)(OR″)OR′″, P(R)(OR′)(OR″)OR′″, P(OR)(OR′)(OR″)OR′, P(O)(R)R′, P(O)(R)OR′, P(O)(OR)OR″, PO₂, S, SR, SOR, S(R)R′, S(OR)R′, S(OR)OR′, S(O), S(R)(R′)R″, S(R)(R′)OR″, S(R)(OR′)OR″, S(OR)(OR′)OR″, S(O)R, S(O)OR, S(R)(R′)(R″)R′″, S(R)(R′)(R″)OR′″, S(R)(R′)(OR″)OR′″, S(R)(OR′)(OR″)OR′, S(OR)(OR′)(OR″)OR′″, S(O)(R)R′, S(O)(R)OR′, S(O)(OR)OR″, SO₂, S(R)(R′)(R″)(R′″)R″″, S(R)(R′)(R″)(R′″)OR″″, S(R)(R′)(R″)(OR′″)OR″″, S(R)(R′)(OR″)(OR′″)OR′, S(R)(OR′)(OR″)(OR′″)OR″″, S(OR)(OR′)(OR″)(OR′″)OR″″, S(O)(R)(R′)R″, S(O)(R)(R′)OR″, S(O)(R)(OR′)OR″, S(O)(OR)(OR′)OR″, SO₂R, SO₂OR, Se, SeR, SeOR, Se(R)R′, Se(OR)R′, Se(OR)OR′, Se(O), Se(R)(R′)R″, Se(R)(R′)OR″, Se(R)(OR′)OR″, Se(OR)(OR′)OR″, Se(O)R, Se(O)OR, Se(R)(R′)(R″)R′″, Se(R)(R′)(R″)OR′″, Se(R)(R′)(OR″)OR′″, Se(R)(OR′)(OR″)OR′″, Se(OR)(OR′)(OR″)OR′″, Se(RO)(R)R′, Se(RO)(R)OR′, Se(O)(OR)OR″, SeO₂, Se(R)(R′)(R″)(R′″)R″″, Se(R)(R′)(R″)(R′″)OR″″, Se(R)(R′)(R″)(OR′″)OR′, Se(R)(R′)(OR″)(OR′″)OR′, Se(R)(OR′)(OR″)(OR′″)OR″″, Se(OR)(OR′)(OR″)(OR′″)OR″″, Se(O)(R)(R′)R″, Se(O)(R)(R′)OR″, Se(O)(R)(OR′)OR″, Se(O)(OR)(OR′)OR″, SeO₂R, SeO₂OR, SSR, SeSeR, SSeR, or SeSR;

Y₁, Y₂, Y₃, Y₄, Y₅, Y₆, Y₇, Y₈ and Y₉ are each independently selected from C, CR, CC(O)R, CC(O)OR, CC(O)N(R)R′, CN(R)R′, N, NR, NC(O)R, or NC(O)OR and Z is R, O, N(R)R′, S, S(O), or SO₂;

R₃, R₄, R_{4′}, R₅, R_{5′}, R₆, R₇ and R_{7′} are each independently selected from —R, —OR, —N(R)R′, —C(O)R, —C(O)OR, —C(O)N(R)R′, a substituted or unsubstituted amino acid, C₁₋₁₂amino alcohol, or C₁₋₁₂carboxylic acid; and

R, R′ R″, R′ and R″″ are each independently H or a substituted or unsubstituted C₁₋₁₀alkyl, C₂₋₁₀alkenyl, C₂₋₁₀alkynyl, C₆₋₁₀aryl, C₃₋₈cycloalkyl, C₄₋₆heterocyclyl or C₅₋₆heteroaryl.

In some embodiments, the compound is a compound of Formula VI and the carbon atom bound to R₃ is bound to an atom selected from the group consisting of Y₁, Y₂, Y₃, Y₄, Y₅, Y₆, Y₇, Y₈ and Y₉.

In some embodiments, the compound is a compound of Formula VII and the carbon atom bound to R₃ is bound to an atom selected from the group consisting of Y₁, Y₂, Y₃, Y₄, Y₅ and Y₆.

Furthermore, the disclosed compounds can also have R₂ being selected from —C(R)═C(R)R′, —C≡CR, or wherein R₂ and X taken together form:

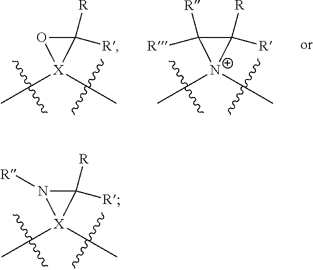

wherein R, R′ R″ and R′″ are each independently H or a substituted or unsubstituted C₁₋₁₀alkyl, C₂₋₁₀alkenyl, C₂₋₁₀alkynyl, C₆₋₁₀aryl, C₃₋₈cycloalkyl, C₄₋₆heterocyclyl or C₅₋₆heteroaryl.

In certain embodiments disclosed herein, the compositions do not include the compounds:

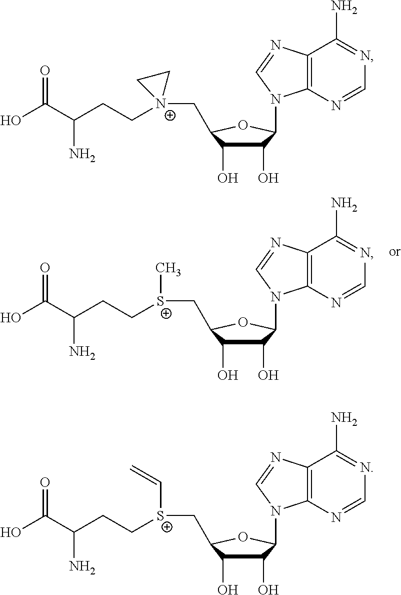

In particular embodiments, the compositions include the compounds:

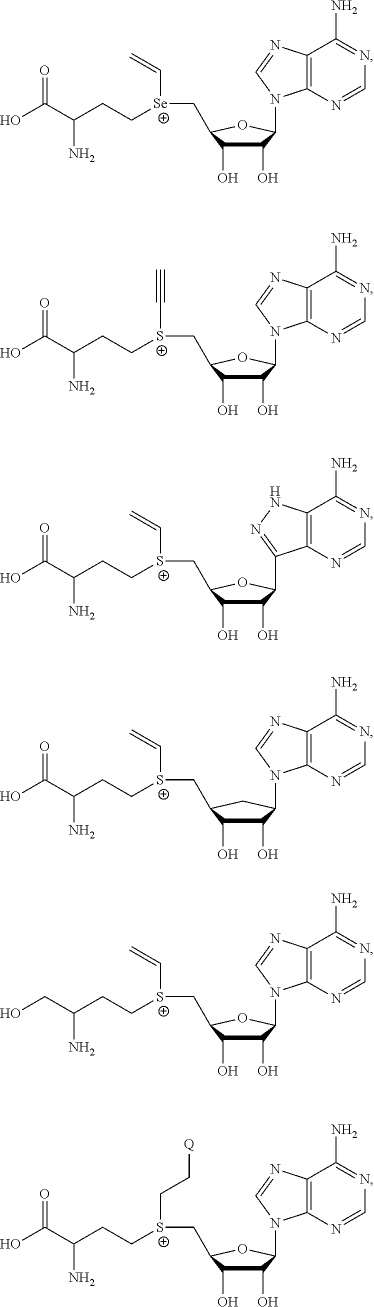

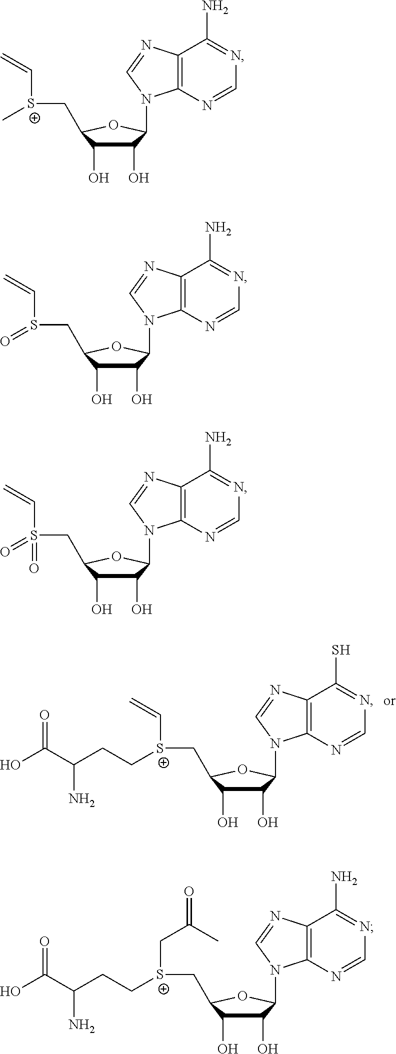

wherein Q is a halogen.

Disclosed herein are compositions comprising a compound of Formula IV:

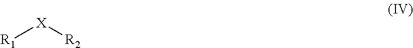

or a salt, hydrate or solvate thereof. In certain aspects, X is C, CR, N, NR, NOR, N(R)OR′, N(OR)OR′, P, PR, POR, P(R)R′, P(OR)R′, P(OR)OR′, P(O), P(R)(R′)R″, P(R)(R′)OR″, P(R)(OR′)OR″, P(OR)(OR′)OR″, P(O)R, P(O)OR, S, SR, SOR, S(R)R′, S(OR)R′, S(OR)OR′, S(O), S(R)(R′)R″, S(R)(R′)OR″, S(R)(OR′)OR″, S(OR)(OR′)OR″, S(O)R, S(O)OR, S(R)(R′)(R″)R′″, S(R)(R′)(R″)OR′″, S(R)(R′)(OR″)OR′″, S(R)(OR′)(OR″)OR′″, S(OR)(OR′)(OR″)OR′″, S(O)(R)R′, S(O)(R)OR′, S(O)(OR)OR″, SO₂, Se, SeR, SeOR, Se(R)R′, Se(OR)R′, Se(OR)OR′, Se(O), Se(R)(R′)R″, Se(R)(R′)OR″, Se(R)(OR′)OR″, Se(OR)(OR′)OR″, Se(O)R, Se(O)OR, Se(R)(R′)(R″)R′″, Se(R)(R′)(R″)OR′″, Se(R)(R′)(OR″)OR′″, Se(R)(OR′)(OR″)OR′″, Se(OR)(OR′)(OR″)OR′″, Se(RO)(R)R′, Se(RO)(R)OR′, Se(O)(OR)OR″, SeO₂, SS, SSe, SeS, or SeSe and R₁ is —R, a substituted or unsubstituted amino acid, C₁₋₁₂amino alcohol, C₁₋₁₂carboxylic acid, —OR, ═O, or R₁ and X taken together are:

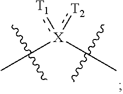

wherein T₁ and T₂ are each independently —OR or ═O; and R₂ is an electrophile. In further embodiments, X and R₂ taken together can form a 3-to-10-membered ring; and R, R′, R″ and R′″ are each independently H or a substituted or unsubstituted C₁₋₁₀alkyl, C₂₋₁₀alkenyl, C₂₋₁₀alkynyl, C₆₋₁₀aryl, C₃₋₈cycloalkyl, C₄₋₆heterocyclyl or C₅₋₆heteroaryl.

In particular embodiments, the compound of Formula IV is selected from:

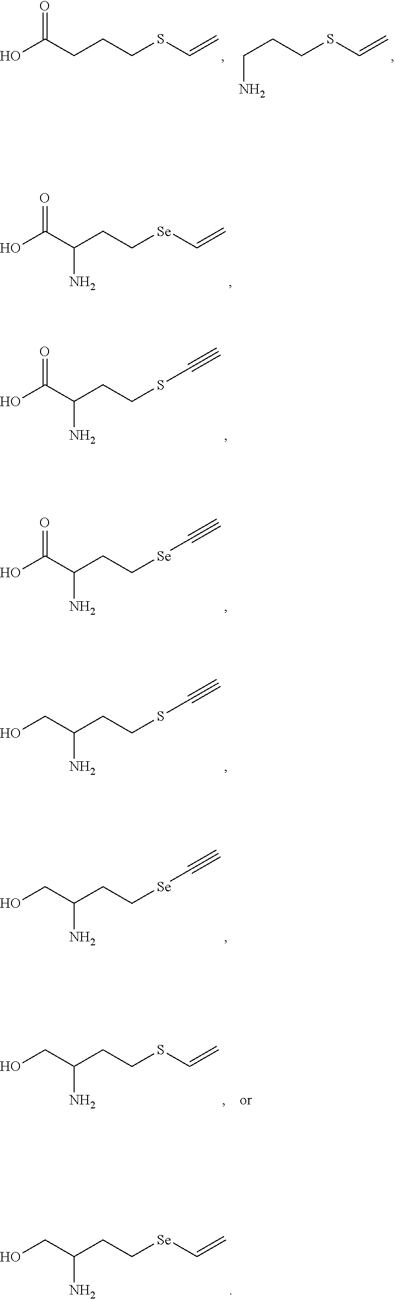

In certain embodiments disclosed herein, the compound of Formula IV is not:

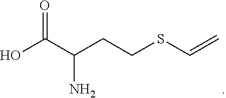

The compositions disclosed herein can further comprise a pharmaceutically acceptable carrier, diluent or excipient. Acceptable diluents are for example physiological salt solutions or phosphate buffered salt solutions Non-limiting examples of such pharmaceutically-acceptable carriers, excipients, and diluents are described in more detail in Remington: The Science and Practice of Pharmacy, Gennaro et al. (eds), 20th Edition, Lippincott Williams & Wilkins, Philadelphia, Pa., 2001 (ISBN 0-683-306472), a standard reference text that is incorporated herein by reference.

Furthermore, the compositions disclosed herein can be delivered to a target by liposomes. Exemplary liposomes include immunoliposomes that incorporate antibodies against cell-specific antigens into liposomes, which carry the compositions (see, e.g., Lasic et al. (1995) Science 267: 1275-76). A number of pre-clinical reports have reported successful targeting and enhanced anti-cancer efficacy with immunoliposomal drugs (Maruyama et al. (1990) J. Pharm. Sci. 74: 978-84); the disclosures of which are incorporated by reference).

### 3. Methods of Use

Disclosed herein are methods of using the disclosed compositions. In some embodiments, a method of inhibiting a methyltransferase is performed. For instance, the method comprises contacting the methyltransferase with a bisubstrate adduct that comprises a methyltransferase substrate and one or more compounds disclosed herein. The compounds disclosed herein can comprise a compound such as:

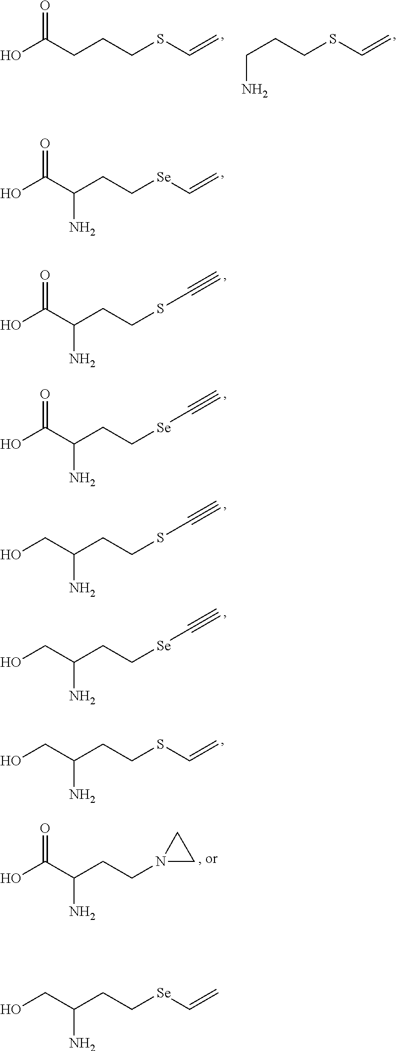

and an adenosine triphosphate. In addition, the bisubstrate adduct inhibits the methyltransferase.

In certain embodiments, the cell is contacted with an AdoMet analog and a compound such as:

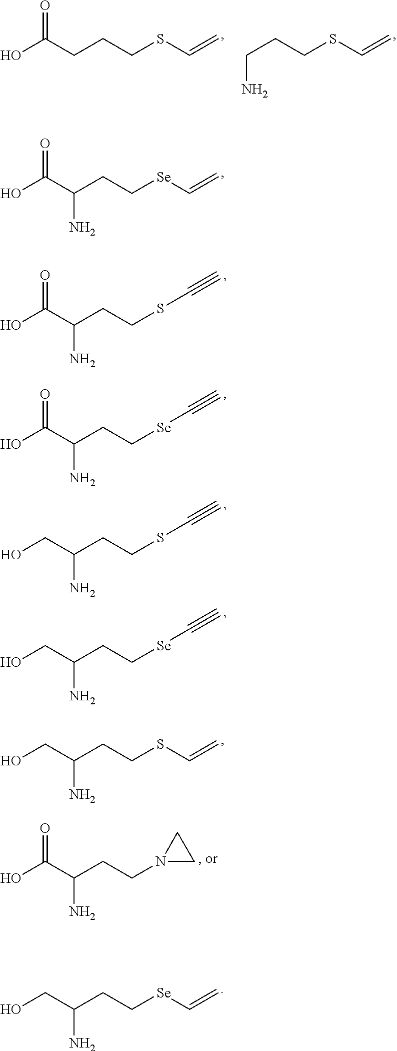

In such embodiments, the compound is generated in the cell by contacting the cell with a compound and a substrate. The compound and the substrate form a bisubstrate adduct in the cell where the methyltransferase exists. In other embodiments, the bisubstrate adduct is formed prior to contacting the cell.

The methods disclosed herein can be used on cells that have been isolated from the tissue or subject of interest. Isolation techniques are well known in the art. Once the cells have been isolated, the compositions can be targeted to the cells for use in the methods. Furthermore, the isolated cells can also be lysed to form lysates. The lysates will comprise active methyltransferase or other AdoMet-binding proteins.

The methods disclosed herein can also be performed in a subject. The subject can be a mammal, fruit fly, or other organism that expresses a methyltransferase protein. In certain embodiments, the compositions are administered in an effective concentration to an mammal systemically, for example, by intravenous, intra-muscular or intraperitoneal administration. Another way of administration comprises perfusion of organs or tissue, be it in vivo or ex vivo, with a perfusion fluid comprising the compositions disclosed herein. The administration may be done as a single dose, as a discontinuous sequence of various doses, or continuously for a period of time sufficient to allow the compositions to perform the functions disclosed herein. In the case of a continuous administration, the duration of the administration may vary depending upon a number of factors that would readily be appreciated by those skilled in the art.

In certain aspects, the method of inhibiting methyltransferase AdoMet-binding proteins, including methyltransferase, treats a disease. In addition, the methods disclosed herein are useful to prevent the uptake of AdoMet into pathogenic organisms to treat a disease. Exemplary diseases associated with methyltransferase other AdoMet-binding proteins, include Parkinson's disease (catechol-O-methyltransferase), tropical parasitic diseases (including diseases caused by Leishmania promastigotes, such as African sleeping sickness and highly tissue destructive disease), fungal infection (including those caused by Candida albicans and various Aspergillus species), Pneumocystis pneumonia, and rheumatoid diseases. The methods disclosed herein can be used to target specific methyltransferases by forming bisubstrate adducts that include the substrate for a particular methyltransferase. In certain embodiments, the substrates include any substrate that allow for methyl transfer reactions. For instance, lipid, a metabolite, a xenobiotic, a drug, or a small molecule can be a substrate. In other embodiments, the methyltransferase substrate is an amino acid, peptide, a protein, a DNA, an RNA, a carbohydrate, a lipid, a metabolite, a xenobiotic, a drug, or a small molecule. The methyltransferase substrate can be a peptide comprising one or more isoaspartyl residues.

Such specificity can be further increased by targeting specific cells that are diseased. Such targeting can be accomplished by the use of liposomes or other agents that target cells.

In other aspects, methods of detecting a methyltransferase substrate are disclosed. The methods comprise contacting a sample comprising the methyltransferase substrate with a compound disclosed herein and a structure such as:

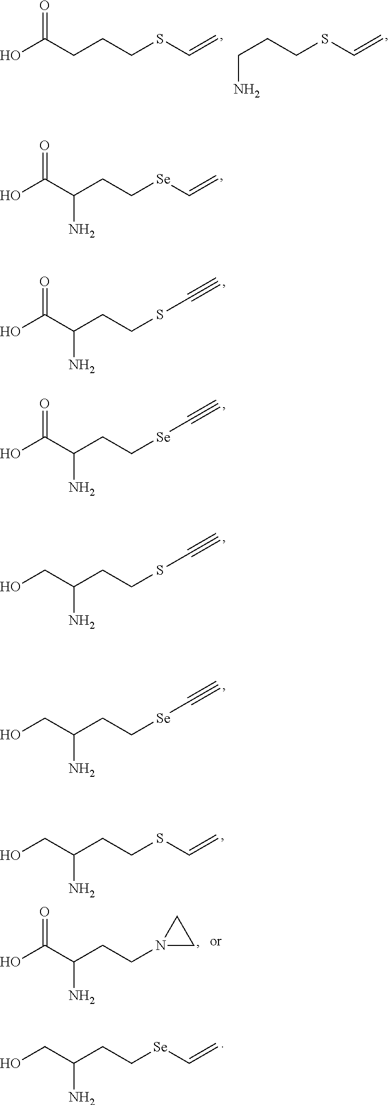

The methods further comprise generating a bisubstrate adduct comprising the AdoMet-binding protein, such as methyltransferase, substrate and the compounds disclosed herein and detecting the methyltransferase substrate in the sample by detecting the bisubstrate adduct in the sample. In such embodiments, the sample includes a cell. The cell can be in a subject, isolated tissue, or isolated individually in a sample. The compound disclosed herein is generated in the cell by contacting the cell with a compound and a substrate. The compound and the substrate form a bisubstrate adduct in the cell where the methyltransferase exists. In other embodiments, the bisubstrate adduct is formed prior to contacting the cell.

In additional embodiments, the compounds disclosed herein are detectably-labeled. As used herein, “detectably labeled” means that a binding agent of the invention is operably linked to a moiety that is detectable. “Operably linked” means that the moiety is attached to the binding agent by either a covalent or non-covalent (e.g., ionic) bond. Methods for creating covalent bonds are known (see general protocols in, e.g., Wong, S. S., Chemistry of Protein Conjugation and Cross-Linking, CRC Press 1991; Burkhart et al., The Chemistry and Application of Amino Crosslinking Agents or Aminoplasts, John Wiley & Sons Inc., New York City, N.Y. 1999). A detectable label is a moiety that can be tracked, and includes, without limitation, fluorophores (e.g., fluorescein (FITC), phycoerythrin, rhodamine), chemical dyes, or compounds that are radioactive, chemoluminescent, magnetic, paramagnetic, promagnetic, or enzymes that yield a product that may be colored, chemoluminescent, or magnetic. In particular embodiments, the detectable label is detectable to a medical imaging device or system. For example, where the medical imaging system is an X-ray machine, the detectable label that can be detected by the X-ray machine is a radioactive label (e.g., ³²P). Note that a binding agent need not be directly conjugated to the detectable moiety. For example, a binding agent (e.g., a mouse anti-human vimentin antibody) that is itself specifically bound by a secondary detectable binding agent (e.g., a FITC labeled goat anti-mouse secondary antibody) is operably linked to a detectable moiety (i.e., the FITC moiety). In certain embodiments, the binding agent is biotin. In further embodiments, the biotin is specifically bound by avidin or a derivative thereof. In certain embodiments, the adenosine triphosphate of the AdoMet analog is a formycin analog or a fluorescent analog. In certain embodiments, the adenosyl group and analogs are covalently linked to a reporter group that can be further derivatized (e.g., alkynes or azides coupled by “click chemistry” to other reporters and detectable labels, see Kobl, H. C. et al. Angewandte Chemie Intl. Ed. 2001, 40(11), 2004-2021. In certain embodiments, the adenosine triphosphate is labeled with one or more of the group consisting of deuterium, tritium, ¹¹C, ¹²C, ¹³C, ¹⁴C, ¹⁶C, ¹⁷O, and ¹⁸O. In other embodiments, compounds such as

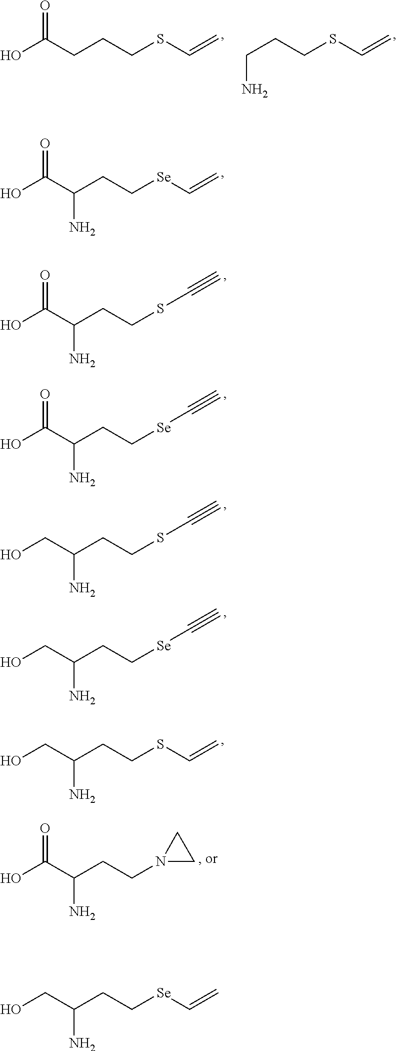

are labeled with one or more of the group consisting of fluorescent labels, deuterium, tritium, ¹¹C, ¹²C, ¹³C, ¹⁴C, ¹⁶O, ¹⁷O, ³²S, ³³S, ³⁴S, ³⁵S, ³⁵S, ⁷²Se, ⁷³Se, ⁷⁴Se, ⁷⁵Se, ⁷⁶Se, ⁷⁷Se, ⁷⁸Se, ⁷⁹Se, ⁸⁰Se, and ⁸²Se.

In certain embodiments, the methods include measuring the amount of methyltransferase substrate in the sample. Standard techniques exist in the art for measuring the amount of methyltransferase. Techniques include fluorescent detection, chemiluminescent detection, and radiolabel detection. In some embodiments, the amount of methyltransferase substrate in the sample is determined by measuring the amount of labeled bisubstrate adduct in the sample. Such methods comprise providing the sample with the compound of Formula I or its component parts. The compound is then bound by the methyltransferase, which further binds to a methyltransferase substrate in the sample.

The methyltransferase forms the bisubstrate adduct, which is can be detected. Detection can be performed if the compound of Formula I, II, III or IV is labeled. Alternatively, it can be performed by direct or indirect immunofluorescence. For example, antibodies can be labeled for detection using chemiluminescent tags affixed to amino acid side chains. Useful tags include, but are not limited to, biotin, fluorescent dyes such as Cy5 and Cy3, and radiolabels (see, e.g., Barry and Soloviev (2000) Proteomics. 4(12): 3717-3726). Tags can be affixed to the amino terminal portion of a protein or the carboxyl terminal portion of a protein (see, e.g., Mattison and Kenney, (2002) J. Biol. Chem., 277(13): 11143-11148; Berne et al., (1990) J. Biol. Chem. 265(32):19551-9). Indirect detection means can also be used to identify the cell markers. Exemplary but non-limiting means include detection of a primary antibody using a fluorescently labeled secondary antibody, or an antibody tagged with biotin such that it can be detected with fluorescently labeled streptavidin.

In certain aspects, methods of isolating a methyltransferase are disclosed. The methods comprise contacting a sample comprising the methyltransferase with a bisubstrate adduct comprising a methyltransferase substrate covalently linked to a compound of Formula I. The compound of Formula I comprising a compound such as

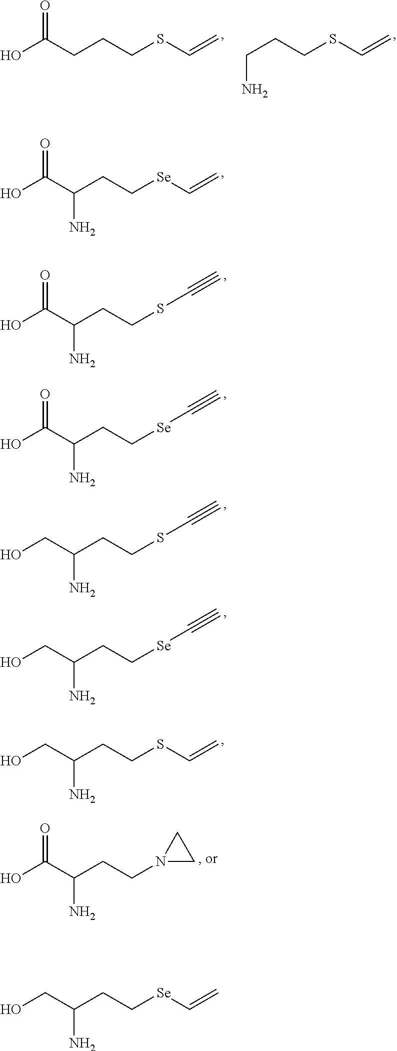

and adenosine triphosphate.

The methods further comprise the sample with the bisubstrate adduct to allow binding of the substrate to methyltransferase and purifying the methyltransferase bound to the bisubstrate adduct from the sample. Standard purification techniques are known in the art. For instance, the purification of methyltransferase can be performed by affinity chromatography, supercritical flow chromatography, and gel electrophoresis. Separation procedures are generally known (see, e.g., Scopes and Scopes, Protein Purification: Principles and Practice, Springer Verlag 1994).

For example, the methyltransferase can be bound to the bisubstrate adduct and isolated by affinity chromatography whereby beads comprising antibodies against the methyltransferase substrate comprise antibodies with affinity for the substrate. The antibodies can be attached to a solid support. Some non-limiting, commonly used support materials include glass, plastics, polystyrene, and metals. Surfaces such as gold, PVDF, silica and polystyrene display high affinities for antibodies (see, e.g., Lal et. al., (2002) DDT (Suppl.) 7(18): S143-S149). The support can be transparent or opaque, flexible or rigid. In some cases, the support is a porous membrane, e.g., nitrocellulose and polyvinylidene difluoride, and the protein capture agents are deposited onto the membrane by physical adsorption. In certain embodiments, the support is a soluble high molecular weight polymer. In certain embodiments, the polymer is polyethylene glycol or a fluorinated polymer.

In addition, chromatography could be used to separate methyltransferase bound to the bisubstrate adduct from other methyltransferase proteins in the sample. This would allow isolation of the methyltransferase of interest. Once the methyltransferase of interest is isolated, the methyltransferase can be separated and thus isolated from the bisubstrate adduct.

In certain embodiments, the methyltransferase is purified by contacting the methyltransferase in a sample of a bisubstrate adduct attached to a solid substrate. The bisubstrate adduct can be disposed on a derivatized solid support utilizing methods practiced by those of ordinary skill in the art (see, e.g., Schena et. al., (1995) Science, 270(5235): 467-470). The bisubstrate adduct can also be “printed” on the solid support. The term “printing”, as used herein, refers to the placement of spots onto the solid support in such close proximity as to allow a maximum number of spots to be disposed onto a solid support. Useful solid supports include, but are not limited to, glass, metal alloy, silicon, and nylon. The support can be a slide derivatized with substances such as aldehydes, epoxies, poly-lysine, silanes, or amines, all of which are well known in the art and provide better deposition of capture probes to the slide.

In other aspects, a method of isolating a methyltransferase substrate is disclosed. The method comprises contacting a sample comprising the methyltransferase substrate and a methyltransferase with a compound of Formula I, which comprises a compound such as

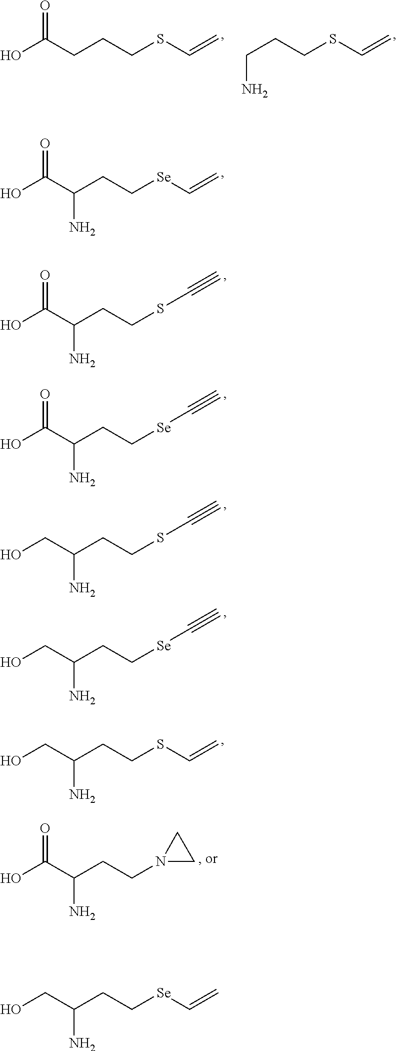

and adenosine triphosphate.

The method further comprises incubating the sample with the compound of Formula Ito allow the methyltransferase to form a bisubstrate adduct comprising the compound of Formula I covalently linked to the methyltransferase substrate. The method further comprises purifying the methyltransferase bound to the bisubstrate adduct from the sample by the techniques disclosed herein. The method also comprises isolating the methyltransferase substrate by cleaving the covalent linkage between the methyltransferase substrate and the compound of Formula I. As described above, purifying the methyltransferase can comprise contacting the methyltransferase bound to the bisubstrate adduct to an antibody attached to a solid support.

For instance, the antibody can be specific for the adenosyl moiety or for the methyltransferase substrate. Useful solid supports include, but are not limited to, glass, metal alloy, silicon, and nylon. The support can be a slide derivatized with substances such as aldehydes, epoxies, poly-lysine, silanes, or amines, all of which are well known in the art and provide better deposition of capture probes to the slide.

Furthermore, the methods disclosed herein relate to increasing and/or maintaining the efficacy of thiopurine. In certain embodiments, the methods comprise administering an effective amount of a compound of Formula I, which comprises a compound such as

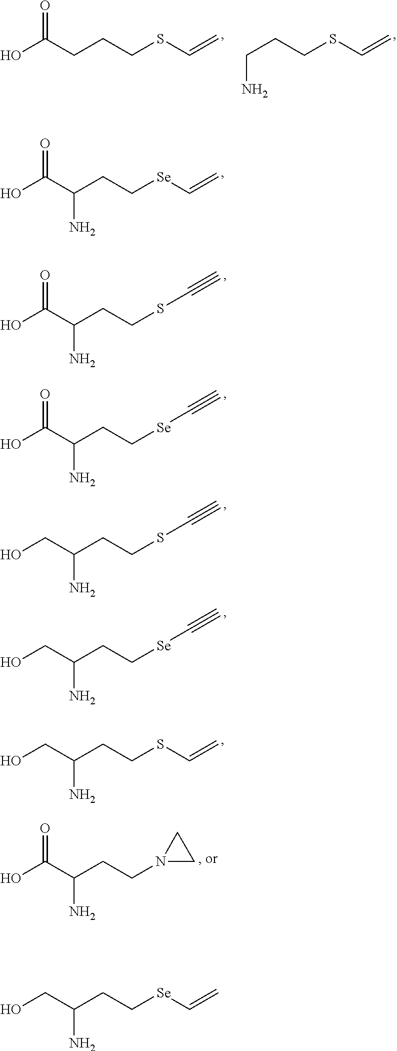

and adenosine triphosphate to a subject who is being administered an effective amount of thiopurine to treat a disease. The administration of the compound of Formula I can be simultaneous to, prior to, and/or subsequent to the administration of thiopurine to the subject. In certain embodiments, the dosage of the compound of Formula I administered to the subject is from about 0.1 mg of compound/kg of subject to about 100 mg of compound/kg of subject. In certain embodiments, the compound of Formula I is administered to the subject in a dosage of about 0.5 mg/kg to about 10 mg/kg. In further embodiments, the compound of Formula I is administered to the subject in a dosage of about 1.0 mg/kg to about 5 mg/kg. Such doses can be provided in concentrations of, for example, about 0.1 ng/mL to about 10.0 g/mL, 10 ng/mL to about 1 g/mL, 100 ng/mL to about 100 mg/mL or about 1 mg/mL to about 10 mg/mL.

### 4. Kits

Further disclosed herein are kits for the analysis (e.g., detection, isolation, and purification) of methyltransferases and their substrates. Such kits can also be used for the inhibition of methyltransferases. The kits can also include the compounds disclosed herein. Such compounds can be stored in any medium that allows for the storage of the compounds. The kits can include methyltransferase substrates disclosed herein. For example, the kits can include proteins comprising one or more isoaspartyl residues for analyses of protein isoaspartylmethyltransferase.

The kits can also include reagents to be used in the analyses described herein. Such reagents include agents for targeting the compositions disclosed herein to particular cells or tissues in a subject. For instance, the kits can include liposomes or immunoliposomes for the targeting of the compositions. In addition, the kits can include reagents for the detection of methyltransferases and their substrates. Such reagents include labeled ATP or any other labeled portion of the compound. Also, the reagents include reagents for detecting the labeled compositions. Such a kit can comprise, e.g., one or more antibodies capable of binding specifically to at least a portion of an methyltransferases and the compositions disclosed herein.

The compositions and reagents can be packaged in a suitable container. The kit can further comprise instructions for using the kit to detect methyltransferases and their substrates.

S-Demethyl-5-vinyl-L-methionine (aka S-vinylhomocysteine or vinthionine) is cell permeable and actively transported into cells where it is enzymatically converted to AdoVin by methionine adenosyltransferase (MAT, or AdoMet synthetase), the S-demethyl-S-vinyl analog of AdoMet that is a frequently substrate for biological methylation reactions. In contrast, AdoMet is not actively transported into mammalian cells. However, AdoMet is actively transported into trypanosomes (Goldberg 1997, Goldberg 1997) and Leishmania promastigotes (Lawrence 1993, Avila 1993), the pathogenic organism responsible for African sleeping sickness and the highly tissue destructive disease, respectively. These related disease organisms do not have completely effective medical cures. AdoMet is also actively transported into Candida albicans and various Aspergillus species that cause fungal infections.

S-Adenosyl-vinthionine (AdoVin), more specifically its vinyl sulfonium functional group, is intrinsically more reactive than AdoMet and its methyl sulfonium group. Additionally, reaction with the vinyl sulfonium is likely to be an electrophilic addition of thiol substrates, catalyzed by methyltransferase enzymes (such as thiopurine methyltransferase (TMPT, EC 2.1.1.67) and homocysteine-5-methyltransferase (HMT, EC 2.1.1.10)), yielding a covalently linked product with the AdoVin. In contrast, AdoMet, generally transfers its methyl group from the alkyl sulfonium moiety in the presence of methyltransferase enzymes and substrates in a group transfer (substitution reaction) that yields two products that dissociate from the enzyme. As a result, methyltransferases catalyze AdoVin coupling to form bi-substrate coupling products that subsequently inactivate the methyltransferase enzymes. Proposed mechanisms for the enzyme-assisted suicide inhibition are the high affinity of the bi-substrate adducts or covalent reactions of the bi-substrate adducts with the methyltransferase enzymes.

In some cases, active uptake pathways for AdoMet exist for some parasites, but not for mammalian cells. Based on this differential uptake, in some embodiments the disclosed AdoVin analogs are used as therapeutic agents for parasitic diseases, as these compounds are selectively taken up by parasites and subsequently inactivate targets in the parasites.

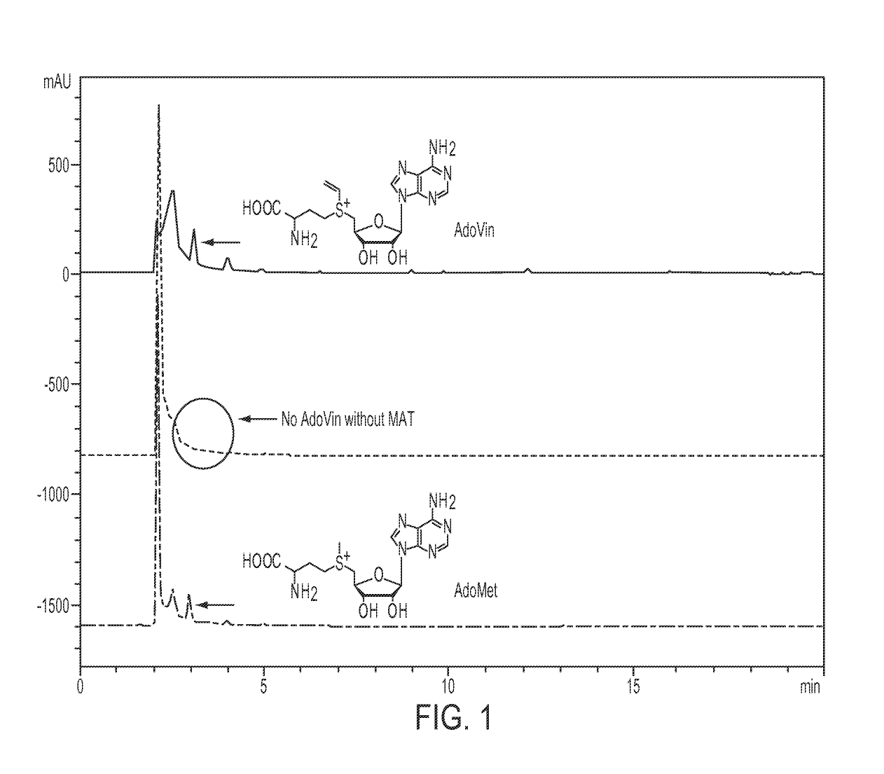

FIG. 1 shows reverse-phase HPLC chromatograms of AdoVin synthesis reactions monitored at 260 nm with 0.1% aqueous TFA at 1 mL/min. The top trace shows an AdoVin synthesis reaction in the presence of MAT. The middle trade shows an AdoVin synthesis reaction in the absence of MAT. The bottom trace shows an AdoMet standard.

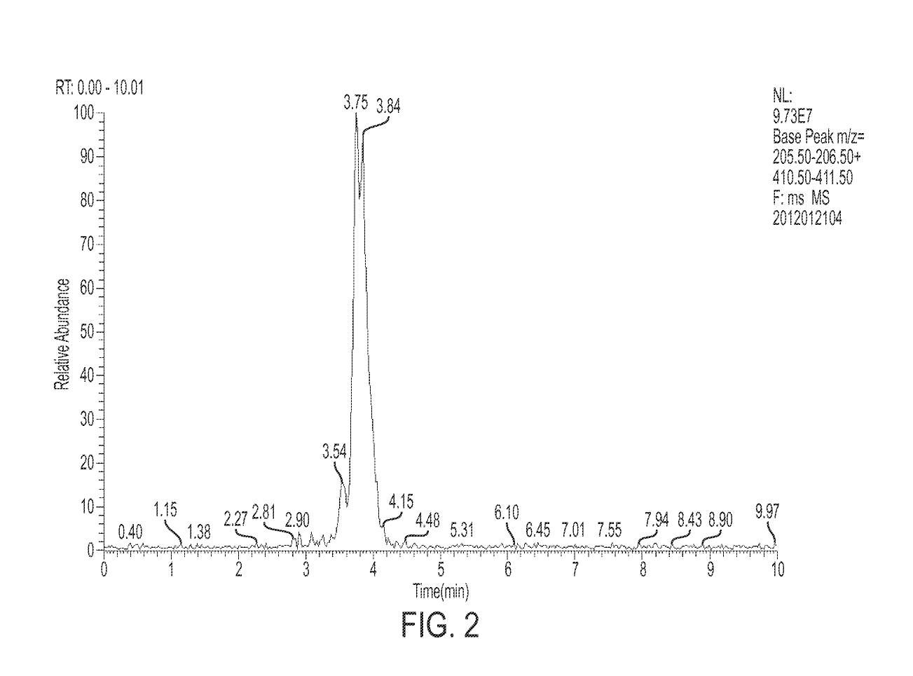

FIG. 2 shows an extracted ion chromatogram of an AdoVin synthesis reaction monitored by reverse-phase HPLC (monitored at 260 nm) eluting with 0.1% aqueous formic acid at a flow rate of 1 mL/min. The two diastereomers at sulfur are not fully resolved under these conditions.

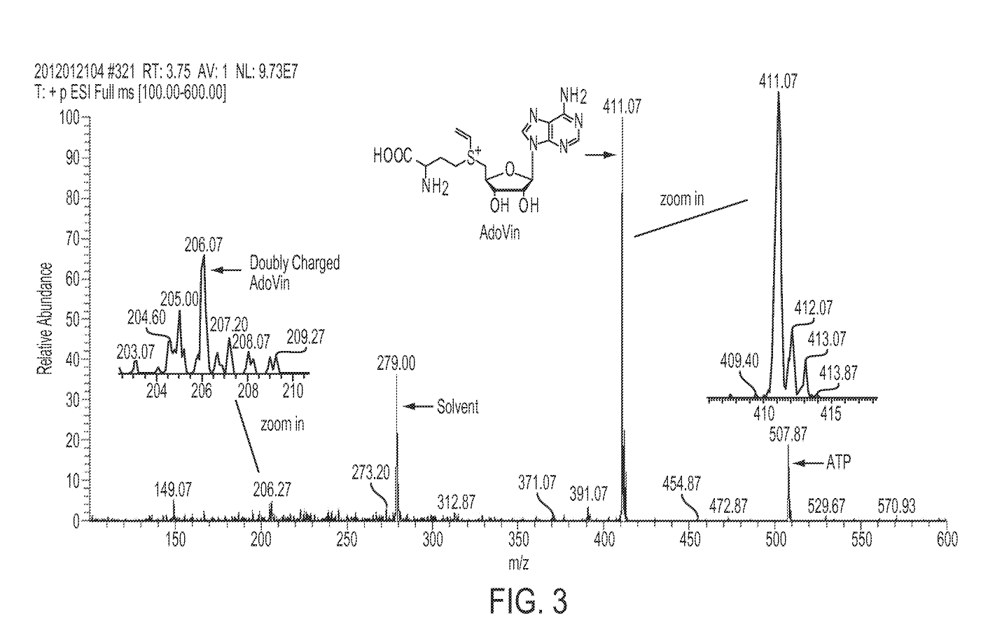

FIG. 3 shows a mass spectrum of an AdoVin synthesis reaction.

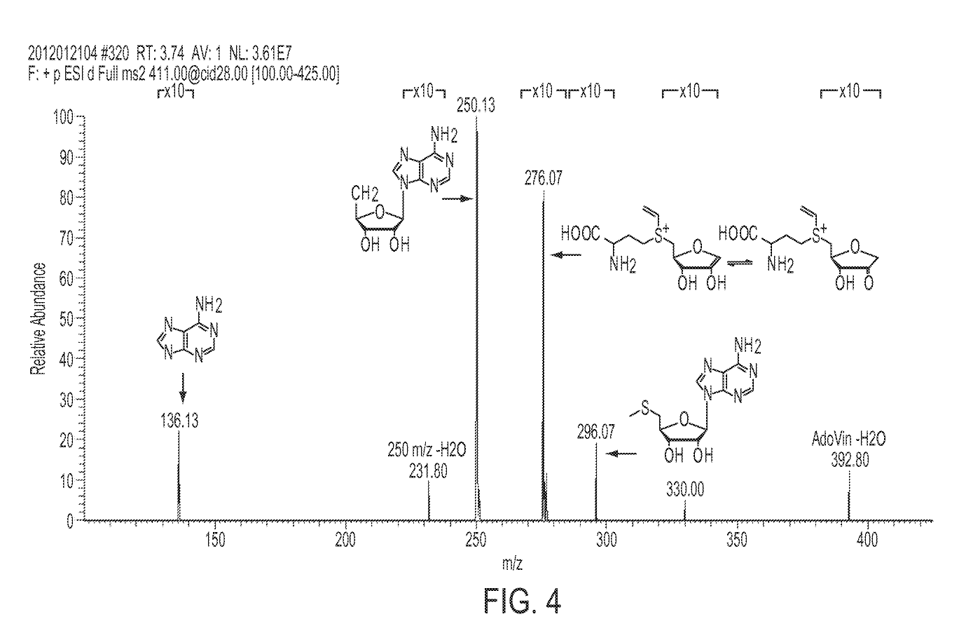

FIG. 4 shows a MS/MS spectrum of the precursor ion of AdoVin.

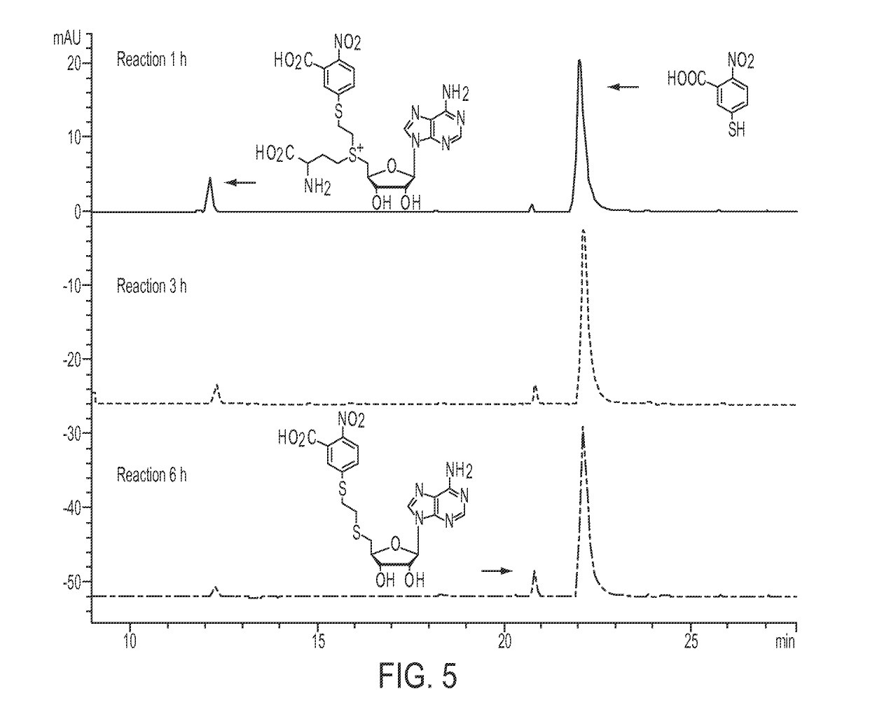

FIG. 5 shows an HPLC chromatogram of a reaction between AdoVin and TNB catalyzed by TPMT at 1, 3 and 6 hours. The reactions were monitored at 350 nm.

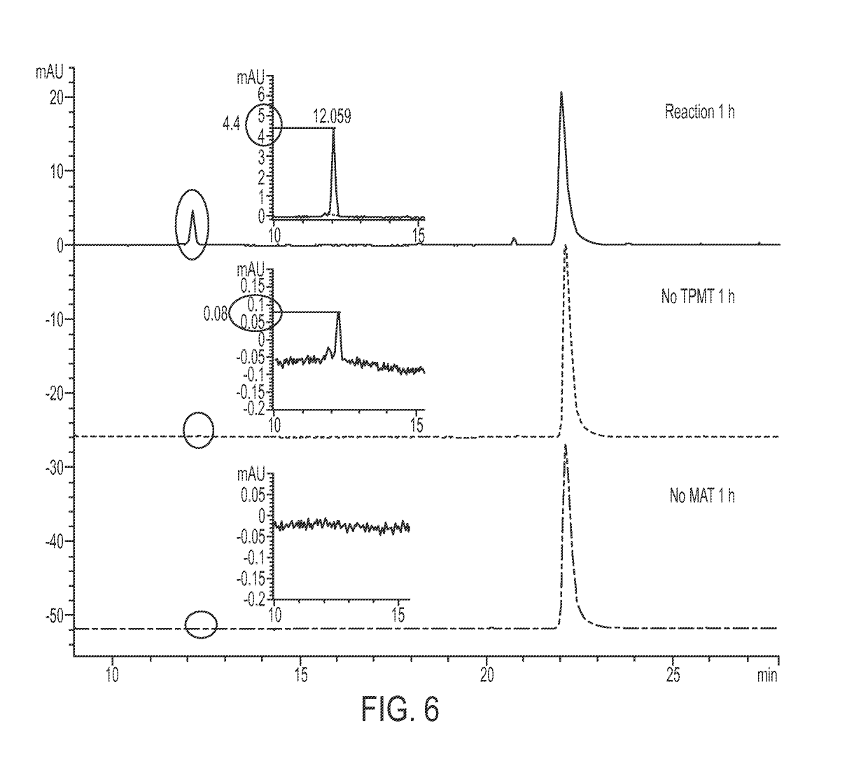

FIG. 6 shows HPLC chromatograms of a reaction between AdoVin and TNB with and without TPMT and without AdoMet synthetase, monitored at 350 nm. The top chromatogram shows a reaction solution containing Vin, MAT, TNB and TPMT after 1 hour. The middle chromatogram shows a reaction solution containing VIN, MAT and TNB after 1 hour. The bottom trace shows a reaction solution containing VIN, TNB and TPMT after 1 hour.

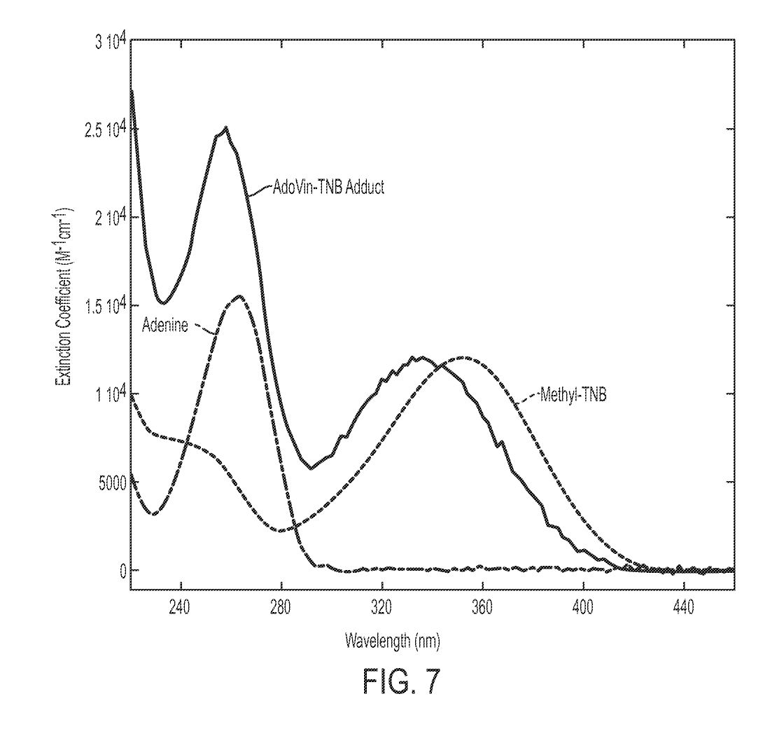

FIG. 7 shows overlayed UV spectra of adenosine (red), methyl-TNB (blue) and AdoVin-TNB (green) adduct. The extinction coefficient of Me-TNB is 12,040 M⁻¹ cm⁻¹. Cannon Anal. Biochem. 2002, 308, 358-363.

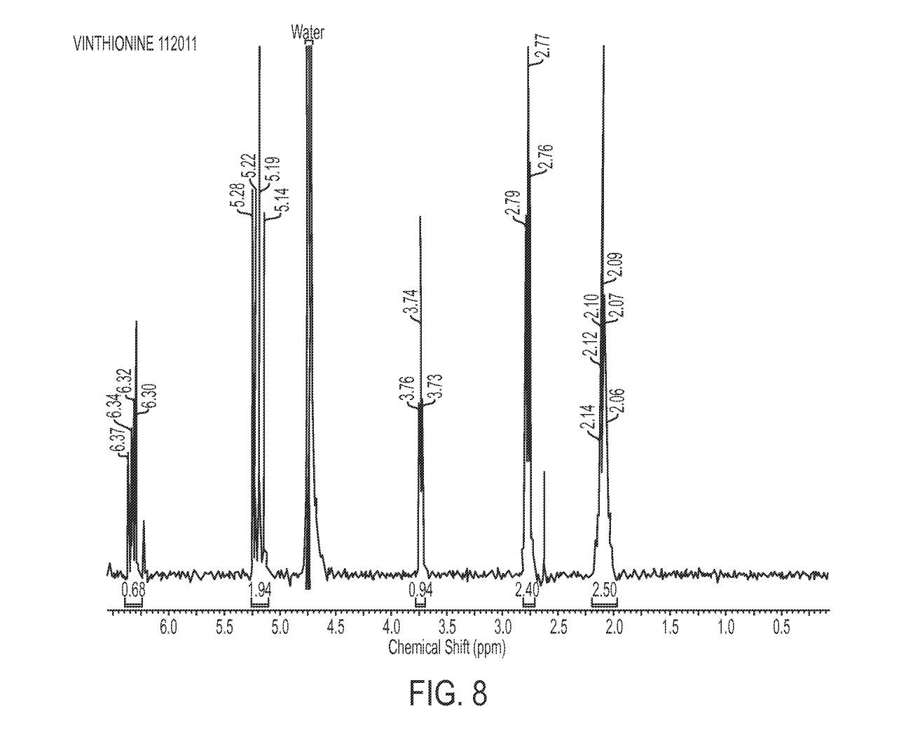

FIG. 8 shows a ¹H Nuclear Magnetic Resonance (NMR) spectrogram of vinthionine.

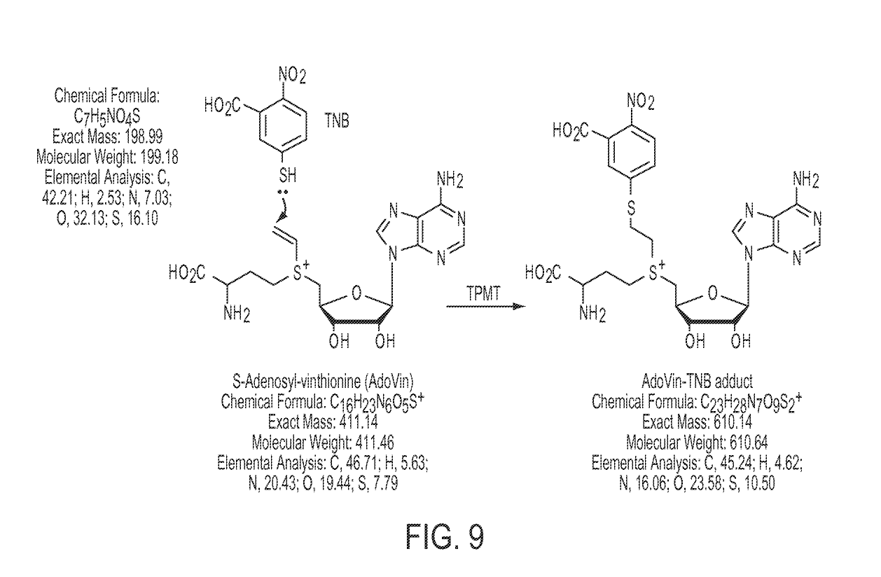

FIG. 9 shows the calculated mass for TNB, AdoVin and their adduct.

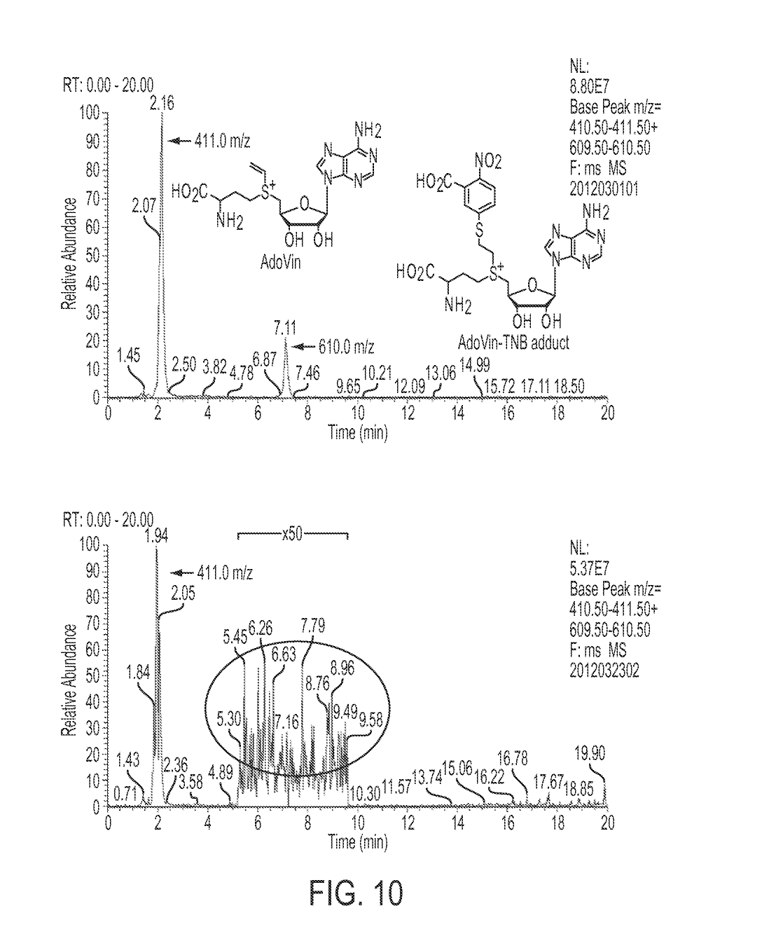

FIG. 10 shows an extracted ion chromatogram (XIC) of reactions between AdoVin and TNB with TPMT (top chromatogram) or without TPMT (bottom chromatogram, negative control).

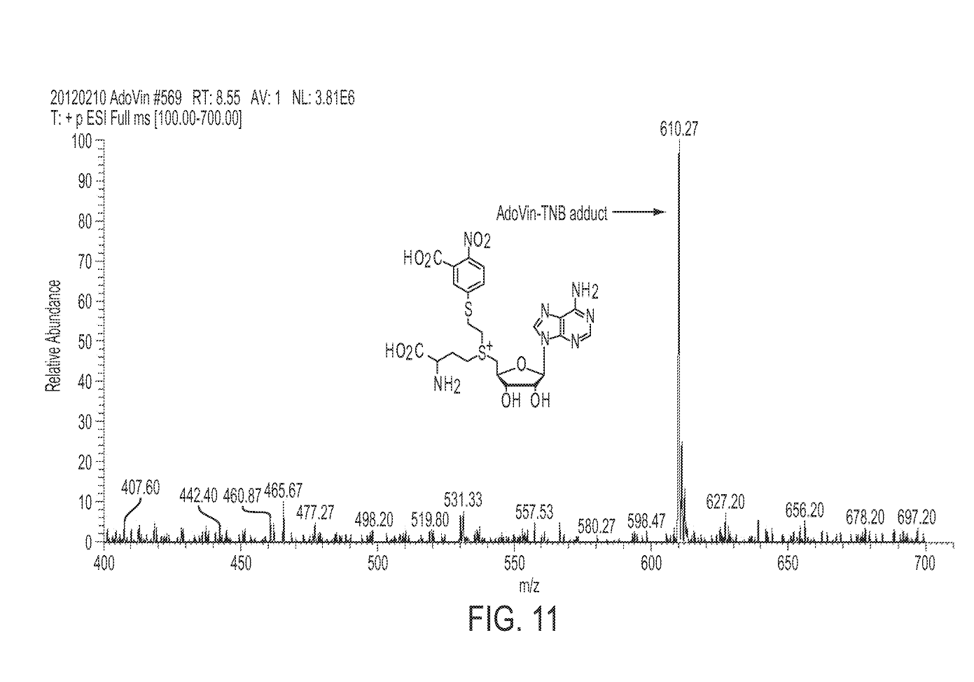

FIG. 11 shows a tandem mass spectrum of the precursor ion of 610.0 m/z and assigned fragmentation pattern of the AdoVin-TNB adduct.

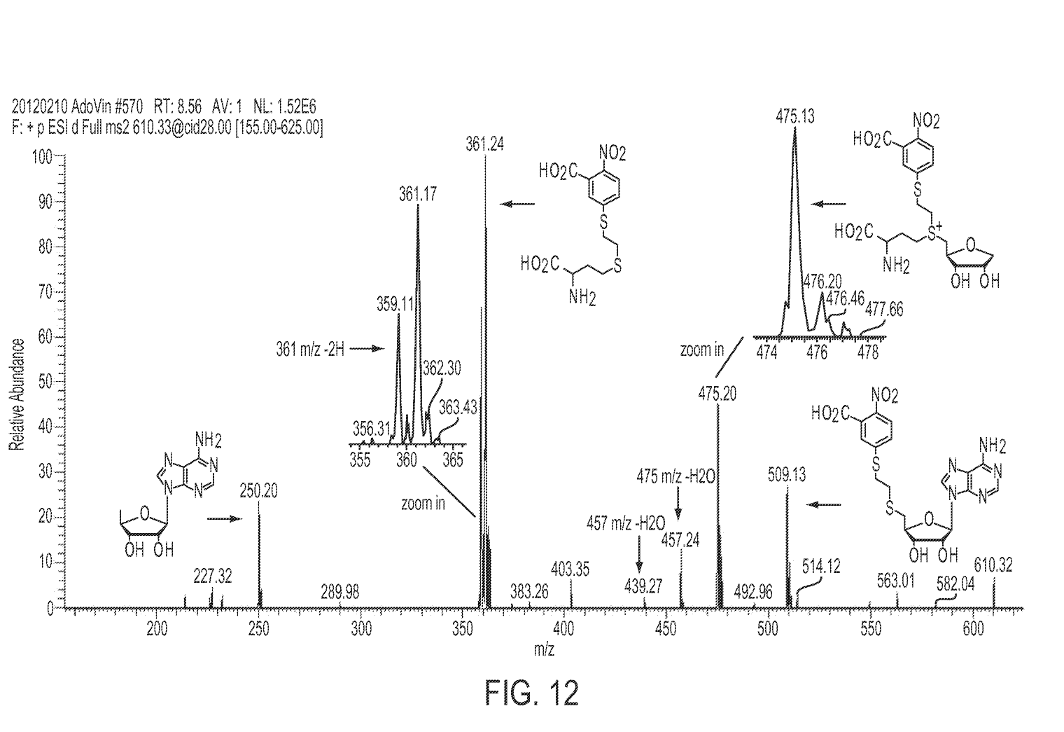

FIG. 12 shows an MS/MS spectrum of the precursor ion of an AdoVin-TNB adduct.

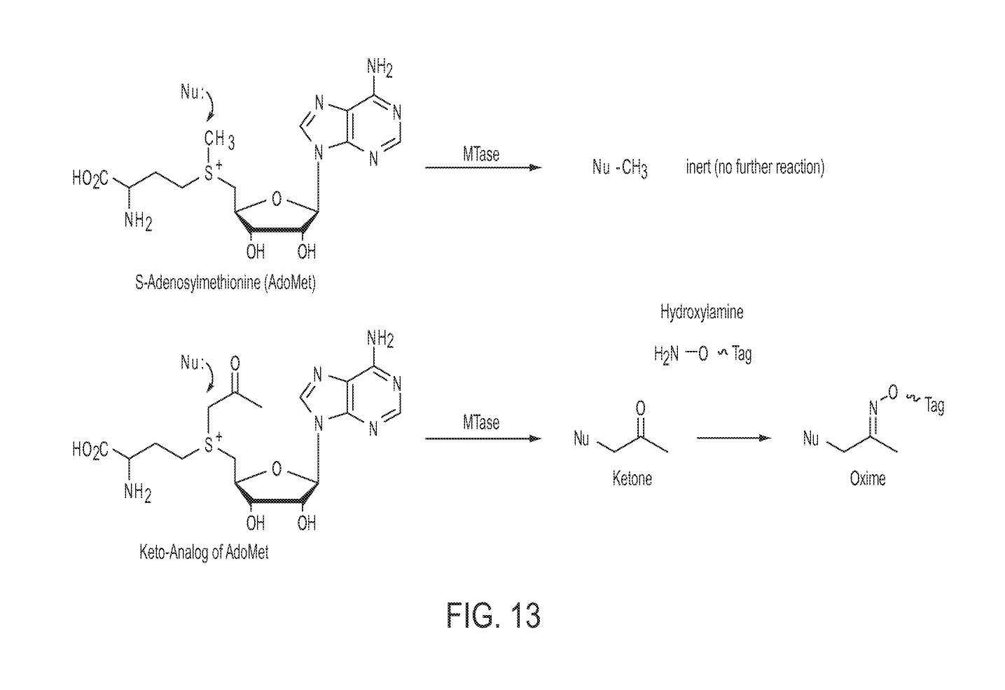

FIG. 13 shows a scheme demonstrating the reactions between methyltransferase and AdoMet and an analog or derivative of AdoMet.

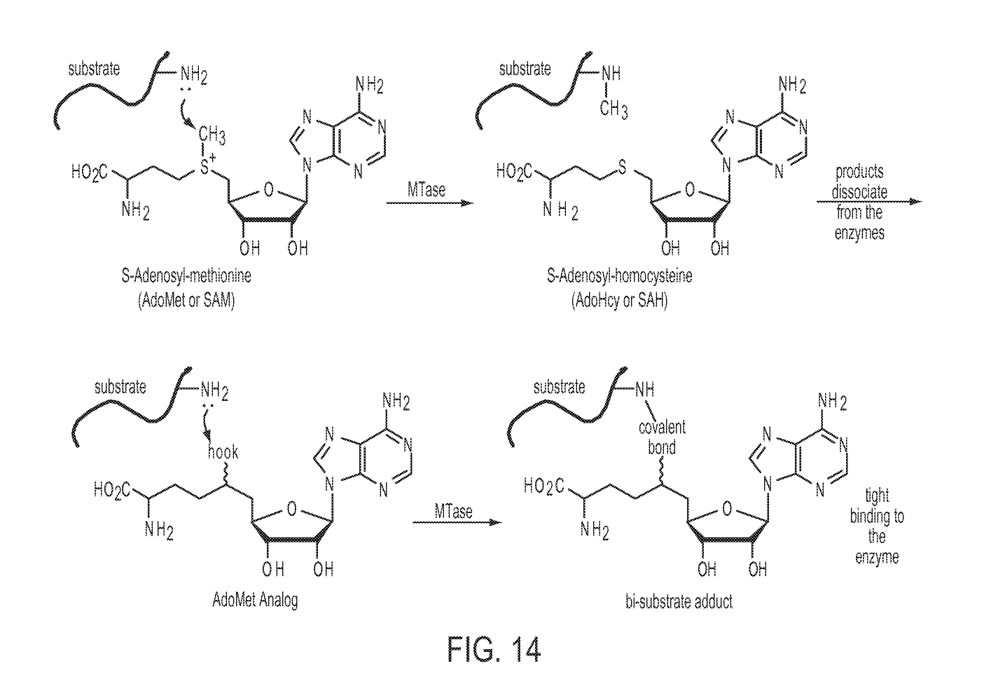

FIG. 14 shows the formation of a bisubstrate adduct for both AdoMet and an AdoMet analog or derivative.

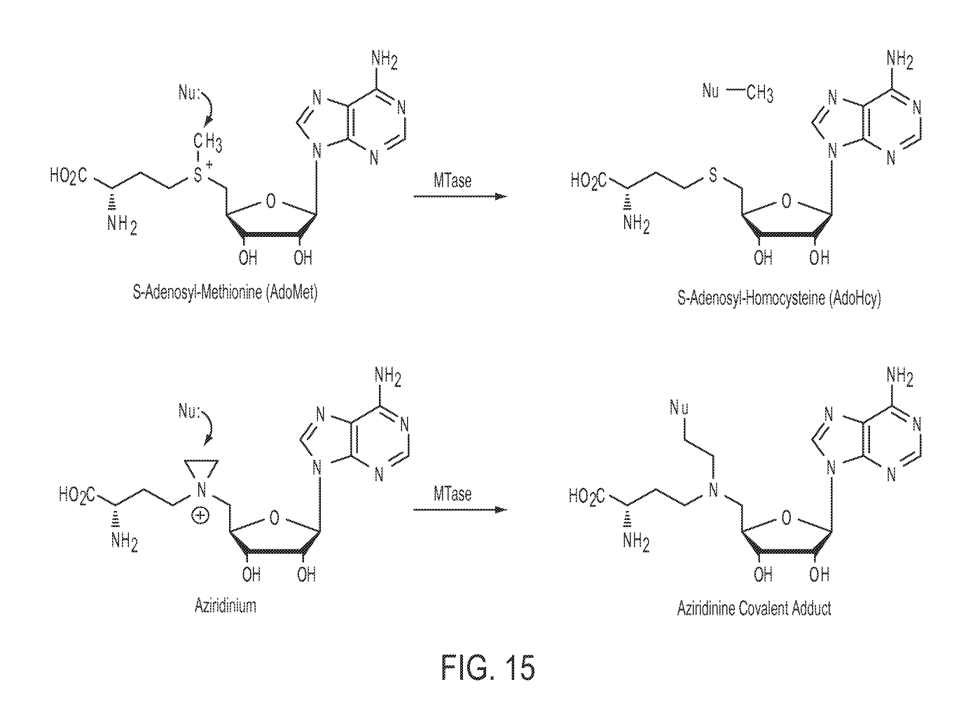

FIG. 15 shows reactions between AdoMet and an AdoMet aziridinium analog or derivative and methyltransferase.

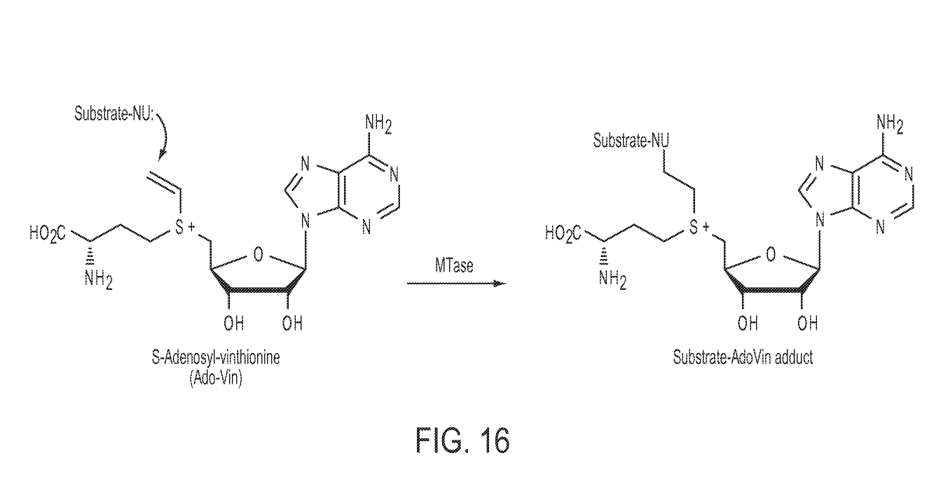

FIG. 16 shows a reaction between methyltransferase and AdoVin to form a bisubstrate adduct.

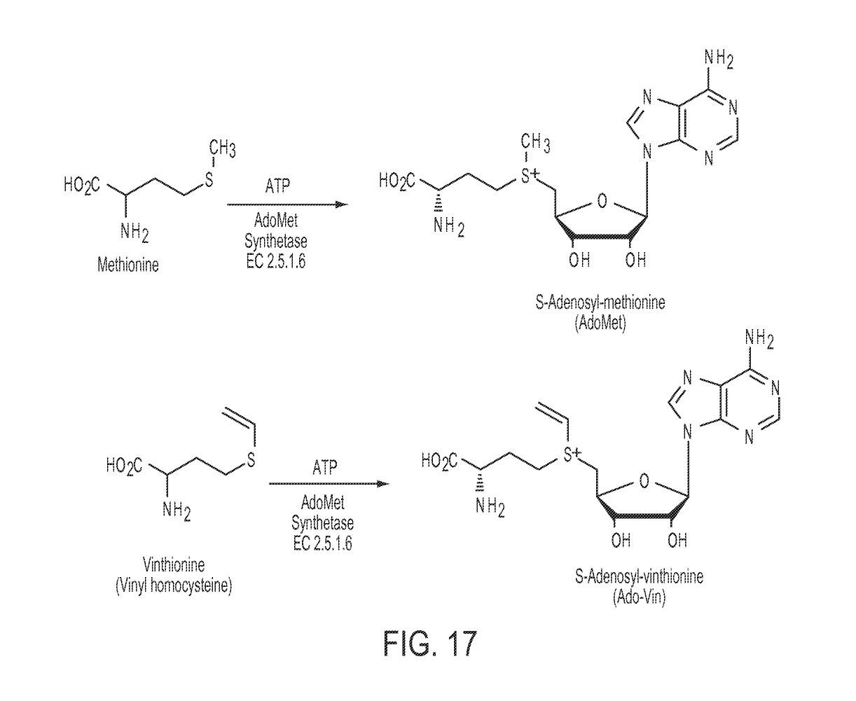

FIG. 17 shows a synthesis of AdoMet from methionine and ATP and AdoVin from vinthionine and ATP catalyzed by AdoMet synthetase.

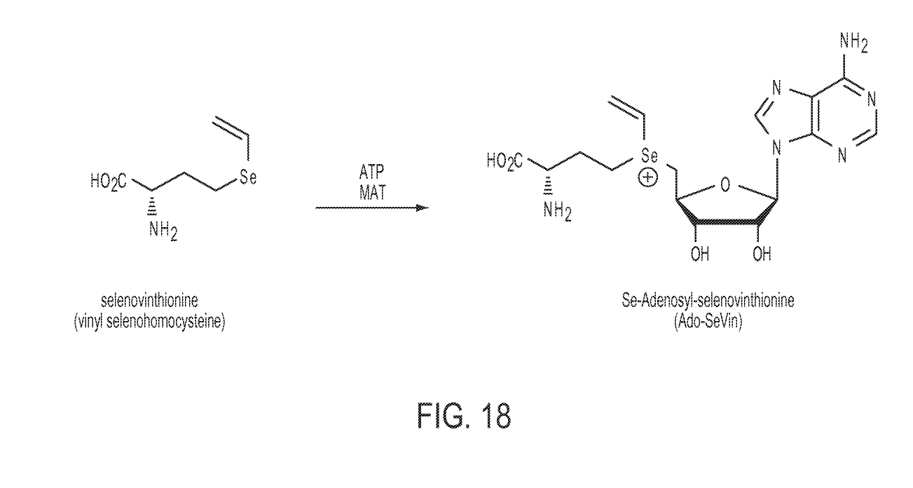

FIG. 18 shows a synthesis of AdoMet from methionine and ATP and AdoVin from vinthionine and ATP, which is catalyzed by AdoMet synthetase (MAT, also known as methionine adenosyltransferase, EC 2.5.1.6).

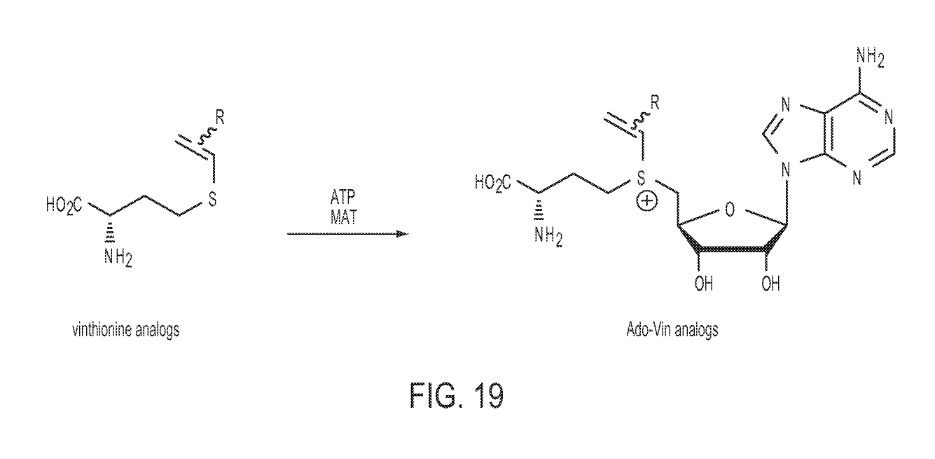

FIG. 19 shows a generic scheme depicting synthesis of AdoVin analogs or derivatives having substituted or unsubstituted alkene groups.

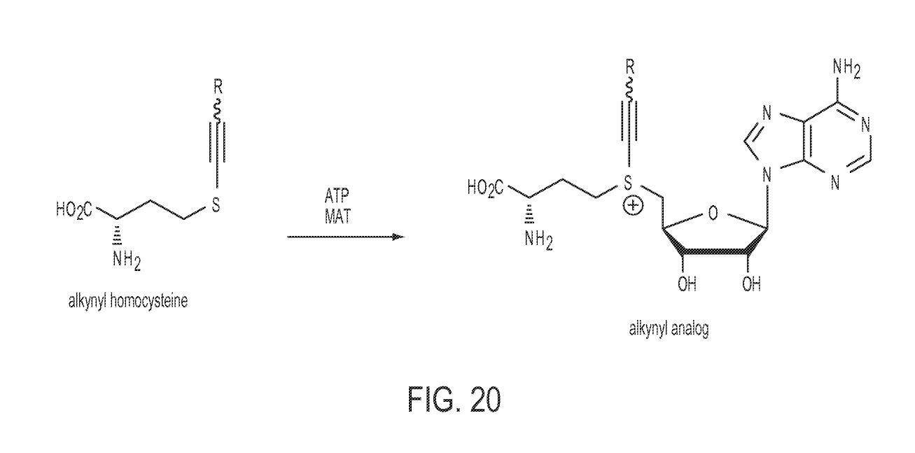

FIG. 20 shows a generic scheme depicting synthesis of AdoVin analogs or derivatives having substituted or unsubstituted alkyne groups.

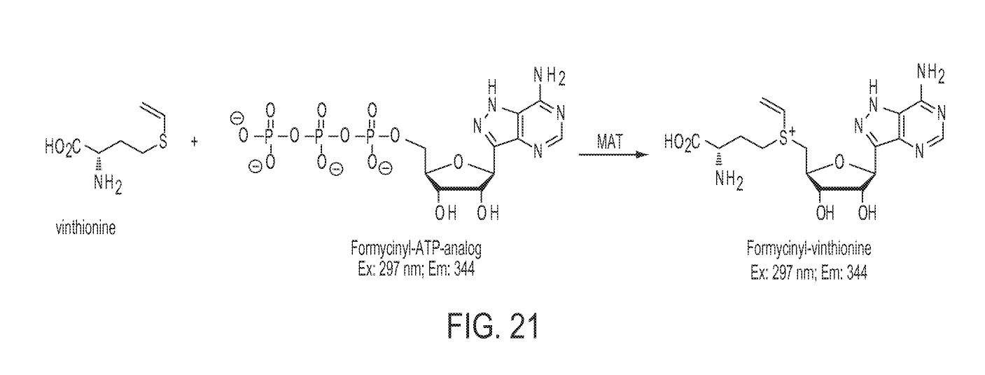

FIG. 21 shows a generic scheme depicting synthesis of AdoVin analogs or derivatives having a formycinyl group.

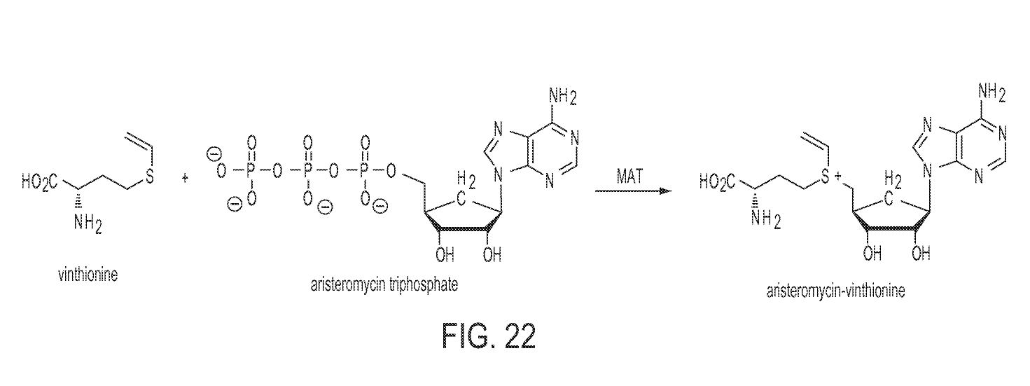

FIG. 22 shows a generic scheme depicting synthesis of AdoVin analogs or derivatives having an aristeromycin group.

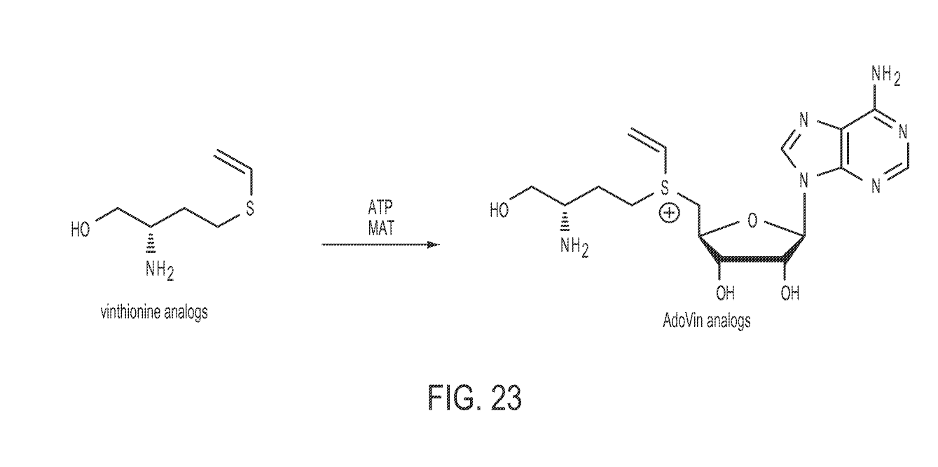

FIG. 23 shows a generic scheme depicting synthesis of AdoVin analogs or derivatives having an amino alcohol group.

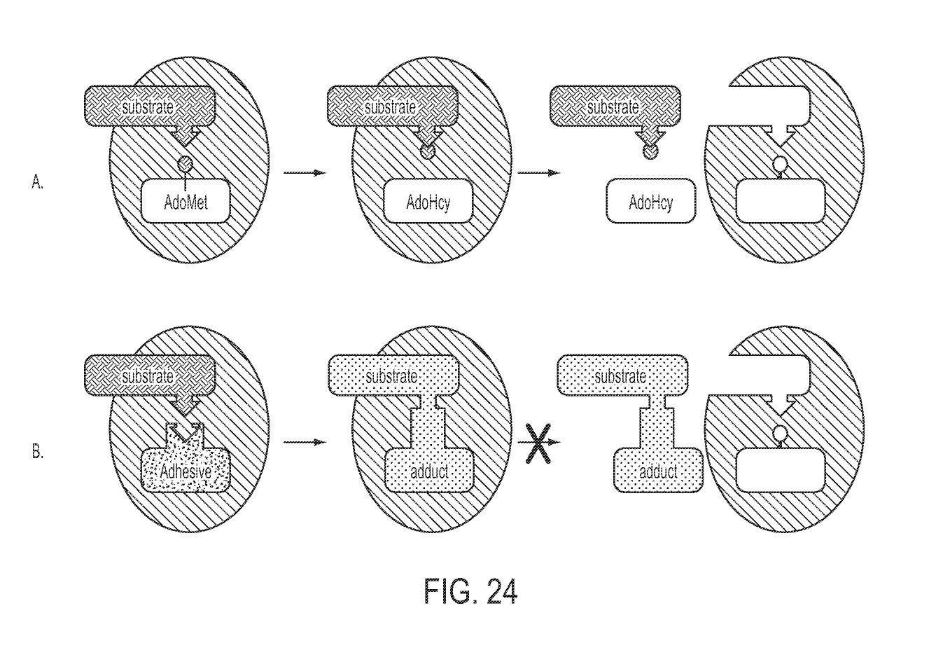

FIG. 24 shows illustrations of transmethylation reactions of AdoMet and AdoVin and its analogs. FIG. 24A illustrates the transfer of a methyl group from AdoMet to a nucleophilic enzyme substrate catalyzed by a methyltransferase (represented by an oval). Subsequently, the methylated products and S-adenosyl-homocysteine (AdoHcy) dissociate from the enzyme. This highlights the transient interaction between a methyltransferase with its substrate and products. FIG. 24B illustrates the initial binding of AdoVin and AdoVin analogs (denoted by “adhesive”) to a methyltransferase, forming covalent adducts with the nucleophilic substrate via addition reactions. The resulting bisubstrate-adducts then bind strongly (with little dissociation) the corresponding methyltransferases with markedly enhanced binding affinity compared to each substrate or product, for the bisubstrate-adducts contain both moieties of the substrates, hence interact with the methyltransferases more extensively and synergistically. As a result, such bisubstrate-adducts inhibit the methyltransferases with high specificity and binding affinity.

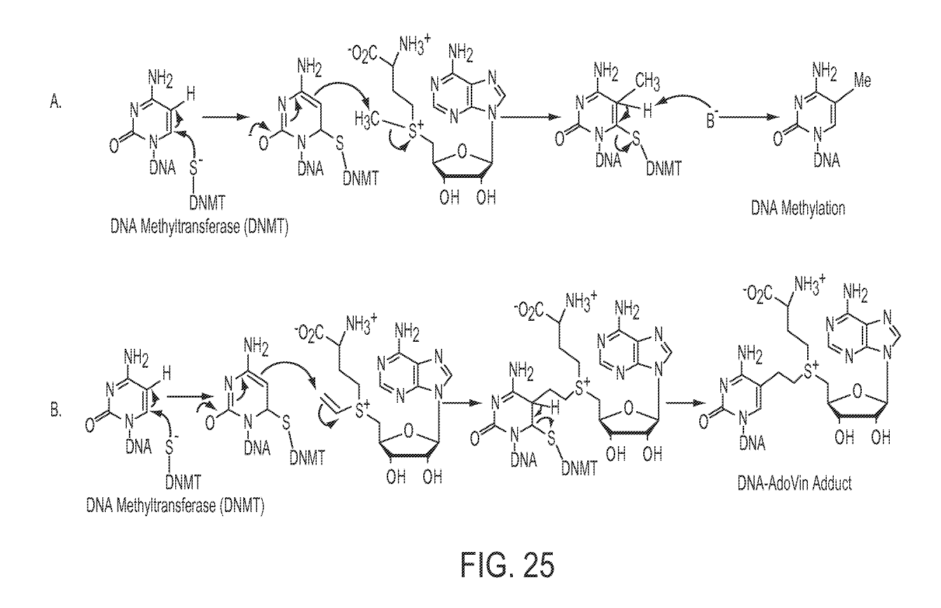

FIG. 25A illustrates the mechanism for the transfer of a methyl group from AdoMet to DNA substrate catalyzed by DNA methyltransferase and the role of the catalytic cysteine in the active site. Siddique 2011 shows that this methylation reaction occurs slowly, as the normal function of the cysteine is to attach the DNA base. FIG. 25B illustrates the mechanism by which AdoVin forms a bisubstrate-adduct with the DNA substrate. Based on the larger size and greater reactivity of AdoVin and its analogs, these compounds are able to covalently modify DNA and proteins. Accordingly, the present disclosure provides for a method of inactivating or modifying AdoMet-binding proteins via direct covalent modification with AdoVin or its analogs.

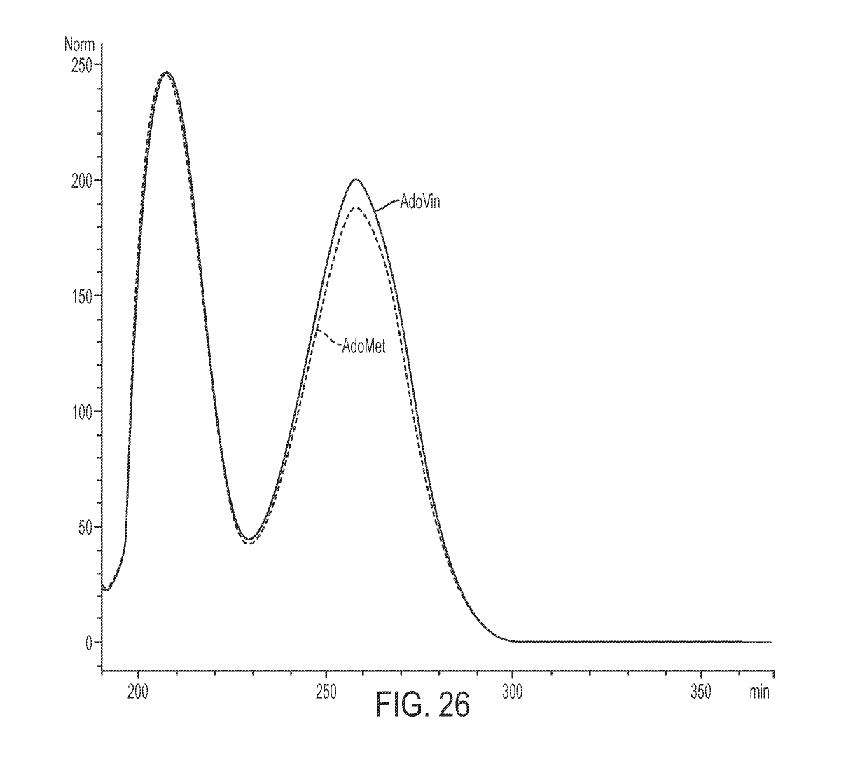

FIG. 26 shows overlaid UV/Vis absorbance spectra of an authentic sample of AdoMet and synthesized AdoVin. As shown in FIG. 26, these spectra correlate closely. The absorbance maximum for AdoVin is 260 nm.

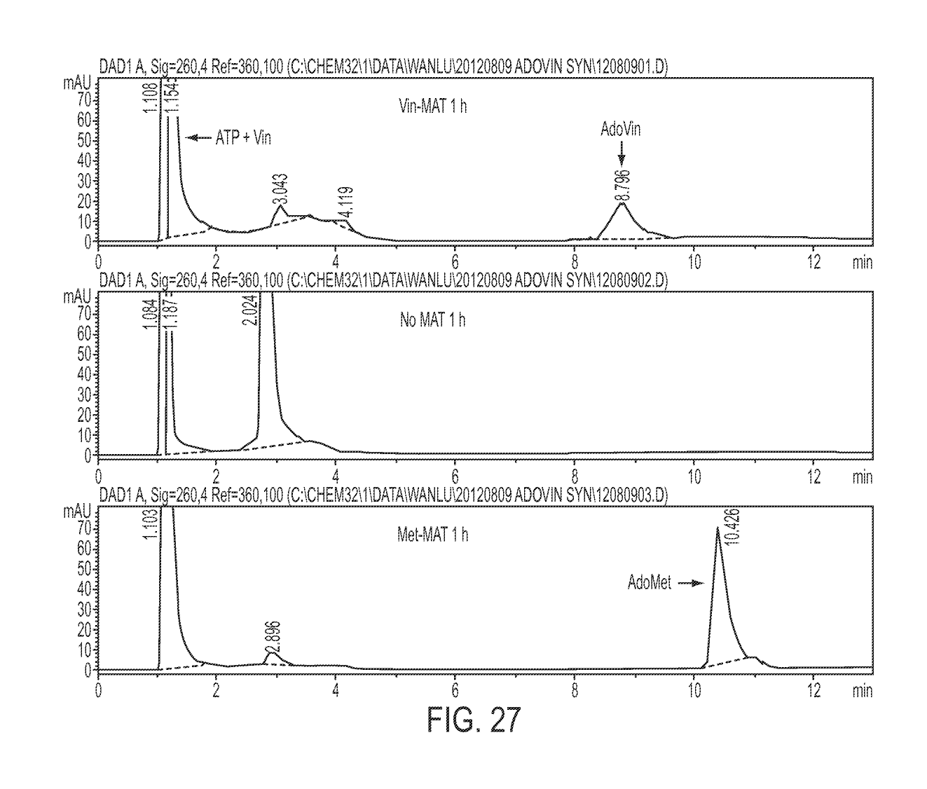

FIG. 27 shows a strong cation exchange (SCX) HPLC chromatogram of AdoVin and AdoMet synthesis reactions. The top trace shows synthesis of AdoVin with MAT, the middle trace shows synthesis of AdoVin without MAT, and the bottom trace shows a synthesis of AdoMet using methionine (“Met”).

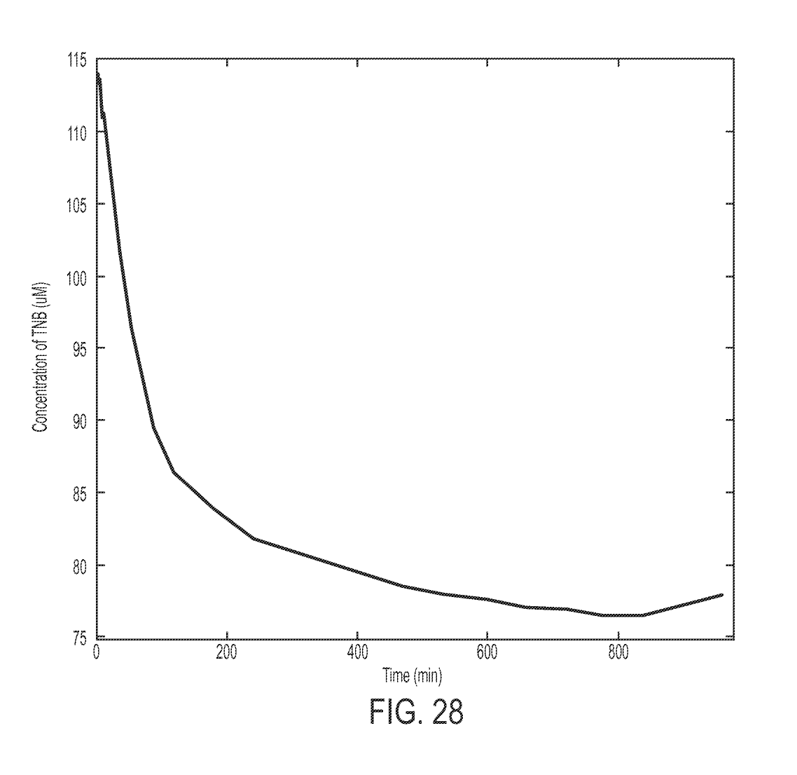

FIG. 28 shows the time-dependent conversion of TNB to a AdoVin-TNB bisubstrate adduct catalyzed by TPMT, as monitored by absorbance changes at 411 nm. The reaction contained 50 mM NH₄HCO₃ (pH 8.0), 10 mM KCl, 4 mM MgCl₂, 2 mM ATP, 400 μM vinthionine (vinyl homocysteine), 22 μM TNB, 23 μM thiopurine methyltransferase (TPMT, EC 2.1.1.67), and were initiated with 79.3 μM S-adenosyl-methionine synthetase (MAT, EC 2.5.1.6) and incubated at 37° C.

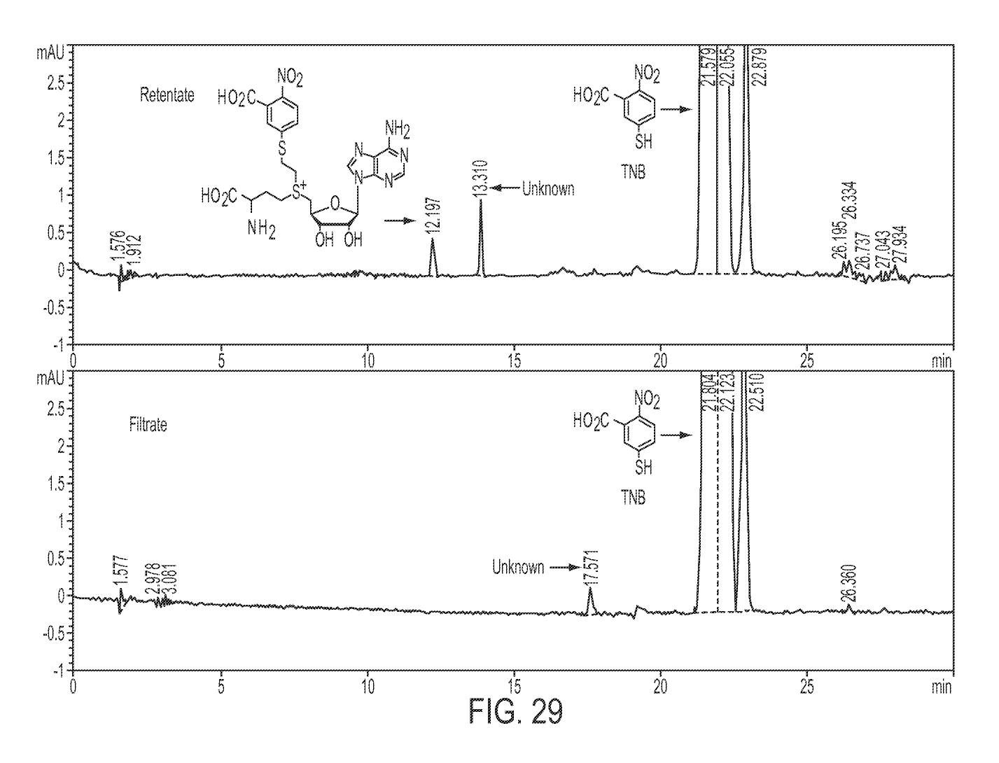

FIG. 29 shows a bisubstrate-adduct and TPMT complex binding assay. The reaction contained 50 mM NH₄HCO₃ (pH 8.0), 10 mM KCl, 4 mM MgCl₂, 2 mM ATP, 300 μM vinthionine (vinyl homocysteine), 166 μM TNB, 83 μM thiopurine methyltransferase (TPMT, EC 2.1.1.67), and were initiated with 42.5 μM S-adenosyl-methionine synthetase (MAT, EC 2.5.1.6) and incubated at 37° C. The reaction sample was filtered using an ultra-filtration centrifugal filter membrane with a molecular weight cutoff of 10 k (Amicon Ultra-2, Pre-launch Centrifugal Filter Devices). The top panel shows the TNB-AdoVin bisubstrate adduct formed at 12.197 minutes, as detected in the enzyme complex. The bottom panel shows that the TNB-AdoVin bisubstrate adduct was not observed in the filtrate.

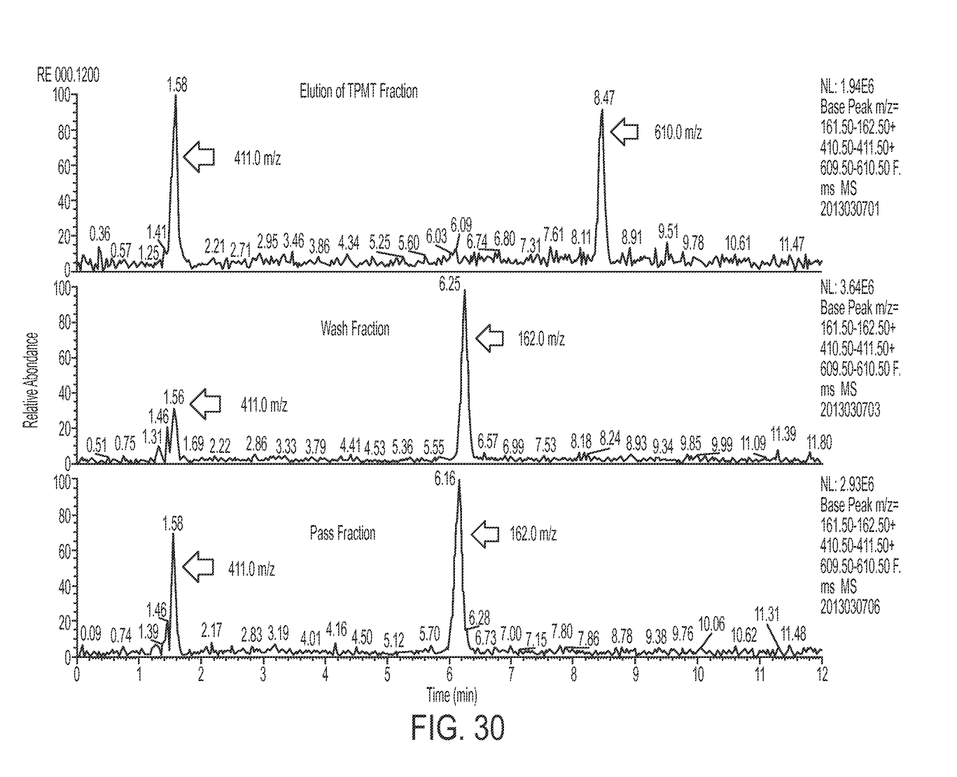

FIG. 30 shows an extracted ion chromatogram (XIC) of AdoVin-TNB binding assays. The histidine-tagged TPMT enzyme was isolated using nickel spin column. 610.0 m/z at 8.6 min is for the TNB-AdoVin adduct (confirmed by tandem mass analysis), 411.0 m/z at 1.6 min is for AdoVin, and 162.0 m/z at 6.2 min is for vinthionine. The top panel shows the TNB-AdoVin bisubstrate adduct at 8.47 min (m/z 611.0) as detected in the enzyme complex. The middle and bottom panels show that the TNB-AdoVin bisubstrate adduct was not observed in the pass and wash fractions. The peak around 1.58 minutes with m/z 411.0 corresponds to AdoVin, while the peak around 6.2 min with m/z 162.0 corresponds to vinthionine. The absence of vinthionine in the top panel indicates that small molecules that do not bind tightly to the TPMT enzyme were washed out of the mixture.

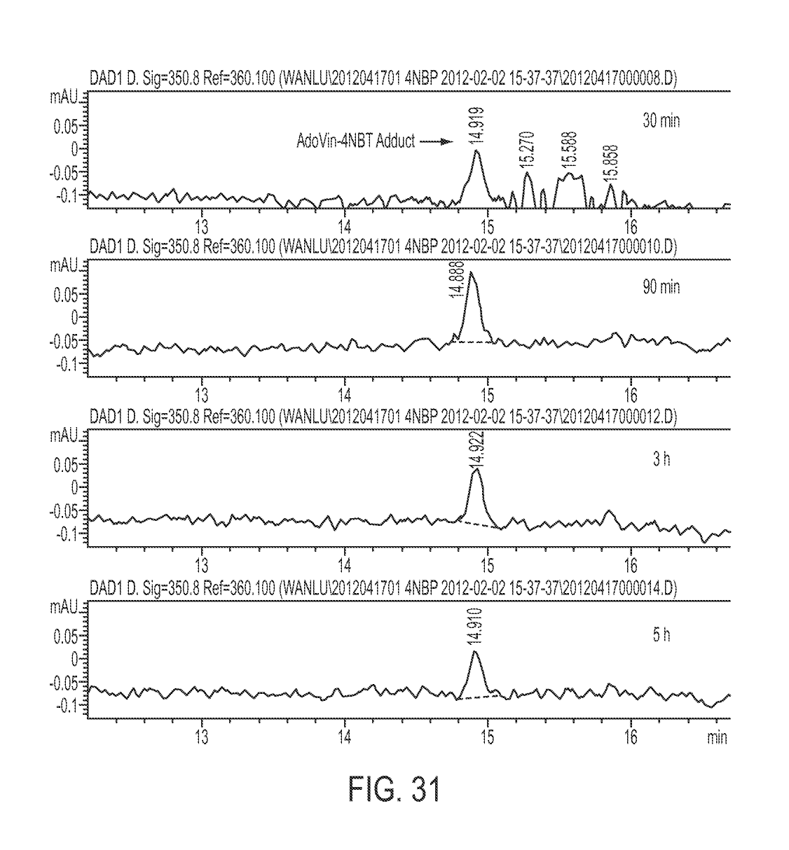

FIG. 31 shows HPLC chromatograms of reactions between AdoVin and 4-nitrobenzenethiol (4-NBT) at 30 minutes, 90 minutes, 3 hours and 5 hours. The reaction contained 50 mM NH₄HCO₃ (pH 8.0), 10 mM KCl, 4 mM MgCl₂, 2 mM ATP, 1.88 mM (tris(2-carboxyethyl)phosphine (TCEP), 4.6 μM 5′-methylthioadenosine nucleosidase (MTAN), EC 3.2.2.9MTAN, 375 μM vinthionine (vinyl homocysteine), 50 μM 4-Nitrobenzenethiol (4-NBT), 50 mM thiopurine methyltransferase (TPMT, EC 2.1.1.67), and were initiated with 64 μM S-adenosyl-methionine synthetase (MAT, EC 2.5.1.6) and incubated at 37° C.

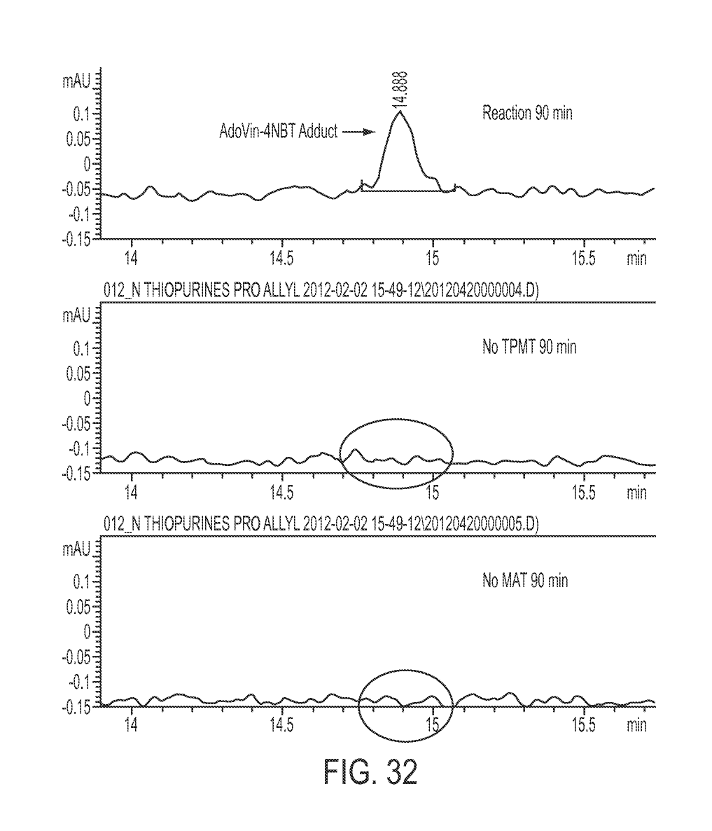

FIG. 32 shows HPLC chromatograms of reactions between AdoVin and 4-nitrobenzenethiol (4-NBT) in the presence and absence of TPMT and MAT as a negative control, monitored at 90 minutes. The top trace shows the presence of the AdoVin-4-NBT adduct when all reagents are present at 90 minutes. The middle trace shows that no AdoVin-4-NBT forms in the absence of TPMT at 90 minutes. The middle trace shows that no AdoVin-4-NBT forms in the absence of MAT at 90 minutes.

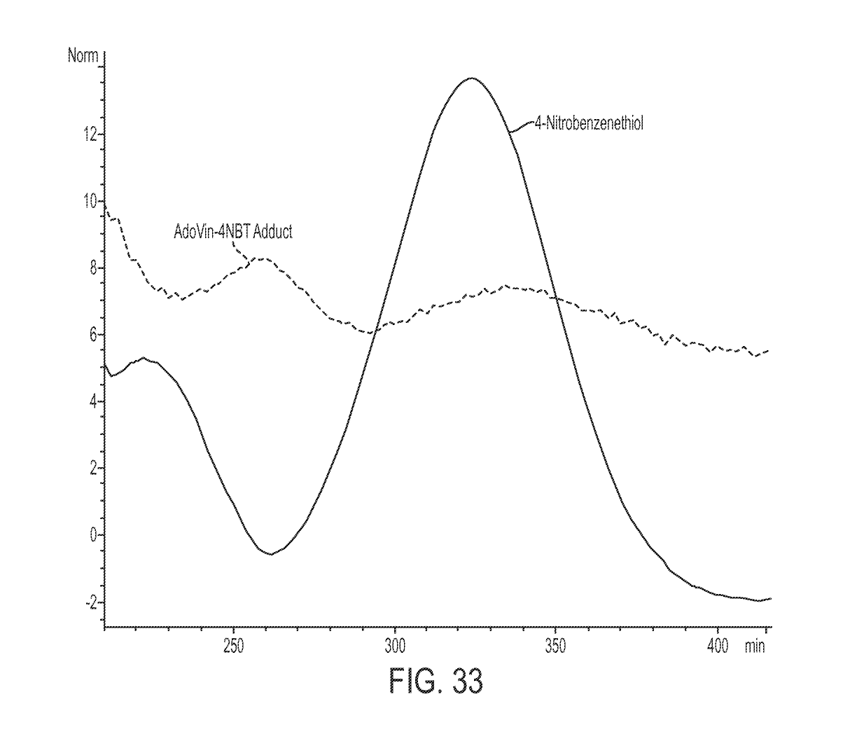

FIG. 33 shows UV spectra of 4-NBT and the AcoVin-4-NBT adduct.

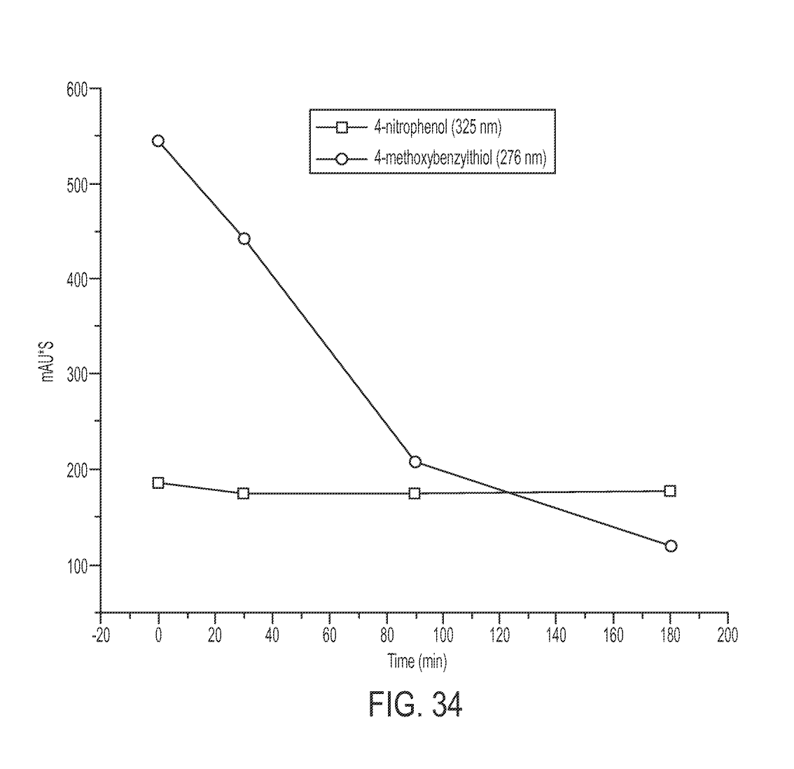

FIG. 34 shows the time-dependent UV absorbance change of 4-methoxybenzylthiol and 4-nitrophenol in a specificity assay with AdoVin. No change was observed for 4-nitrophenol, indicating it does not react with AdoVin. This is consistent with data showing that 4-nitrophenol also does not react with AdoMet (the natural substrate for TPMT). In comparison, the concentration of 4-methoxybenzylthiol decreased in the presence of AdoVin, indicating a reaction with AdoVin. This is consistent with data demonstrating that 4-methoxylbenzylthiol reacts with AdoMet. Taken together, these data suggest that AdoVin displays similar substrate specific to AdoMet.

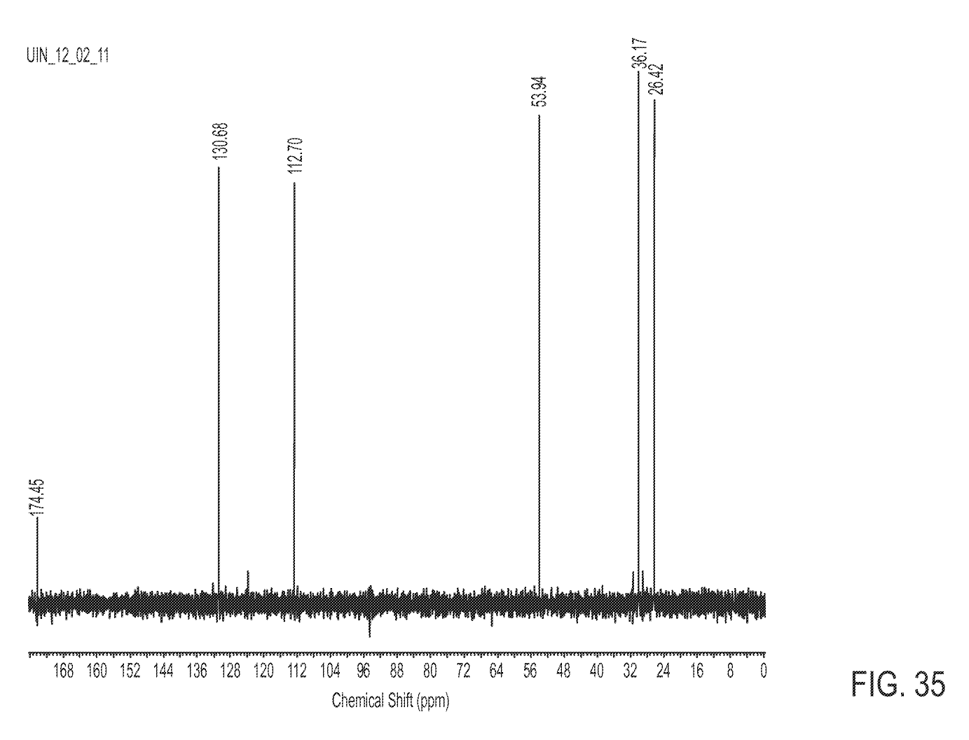

FIG. 35 shows a ¹³C Nuclear Magnetic Resonance (NMR) spectrogram of vinthionine.

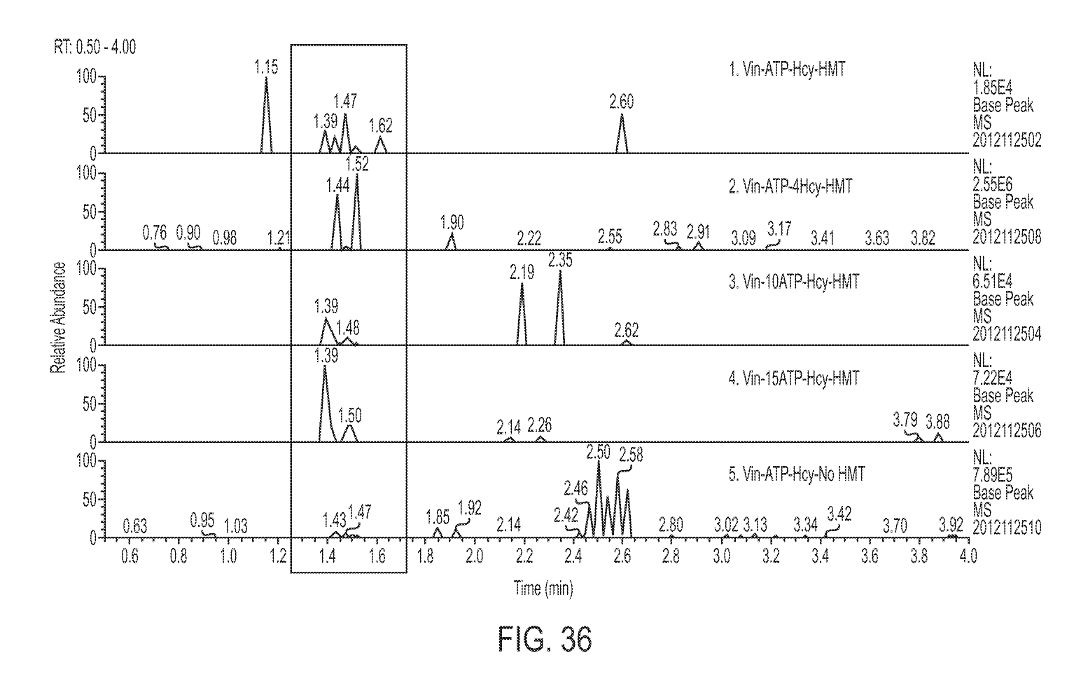

FIG. 36 shows a selected ion monitoring (XIC) chromatogram of m/z 546 for the expected AdoVin-homocysteine (Hcy) adduct, at 1.47 min. Chromatogram 1 shows a reaction between AdoVin and ATP (natural isotopes) in the presence of homocysteine methyltransferase (HMT). Chromatogram 2 shows a reaction between DL-homocysteine-3,3,4,4-D₄ (D denotes deuterium) labeled AdoVin and ATP (natural isotopes) in the presence of HMT. Chromatogram 3 shows a reaction between AdoVin and ¹³C₁₀-labeled ATP with HMT in the presence of HMT. Chromatogram 4 shows a reaction between AdoVin and ¹³C₁₀,¹⁵N₅ labeled ATP in the presence of HMT. Chromatogram 5 shows a reaction between AdoVin and ATP (natural isotopes) in the absence of HMT. These results suggest that the enzyme-catalyzed adduct formation may generate specific stereoisomers that were different than those from the non-enzymatic reactions.

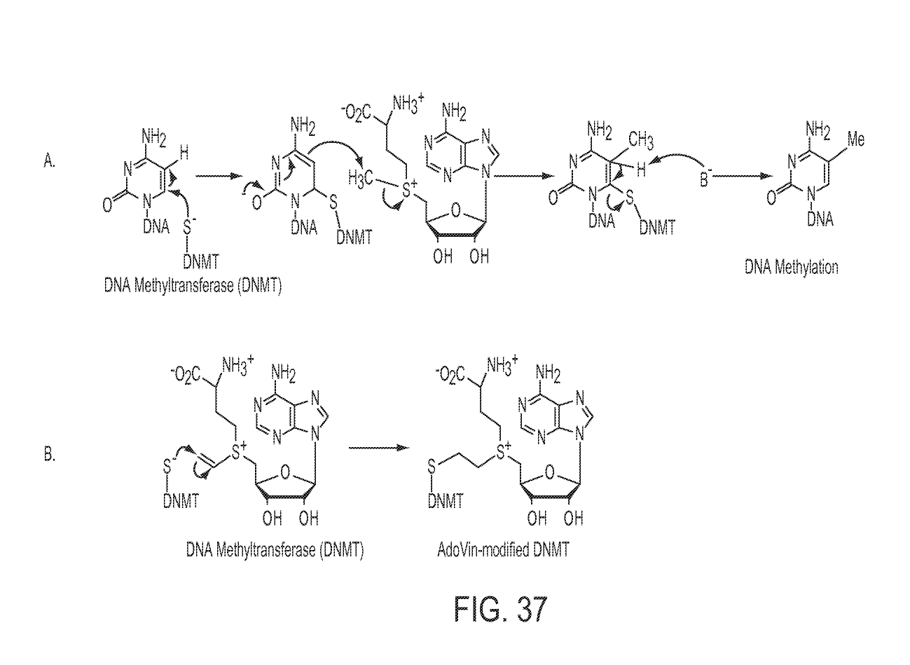

FIG. 37A illustrates the transfer of methyl group from AdoMet to DNA substrate catalyzed by DNA methyltransferase and demonstrates the role of the catalytic cysteine in the active site. FIG. 37B illustrates the direct covalent modification of the catalytic cysteine in the active site by AdoVin.

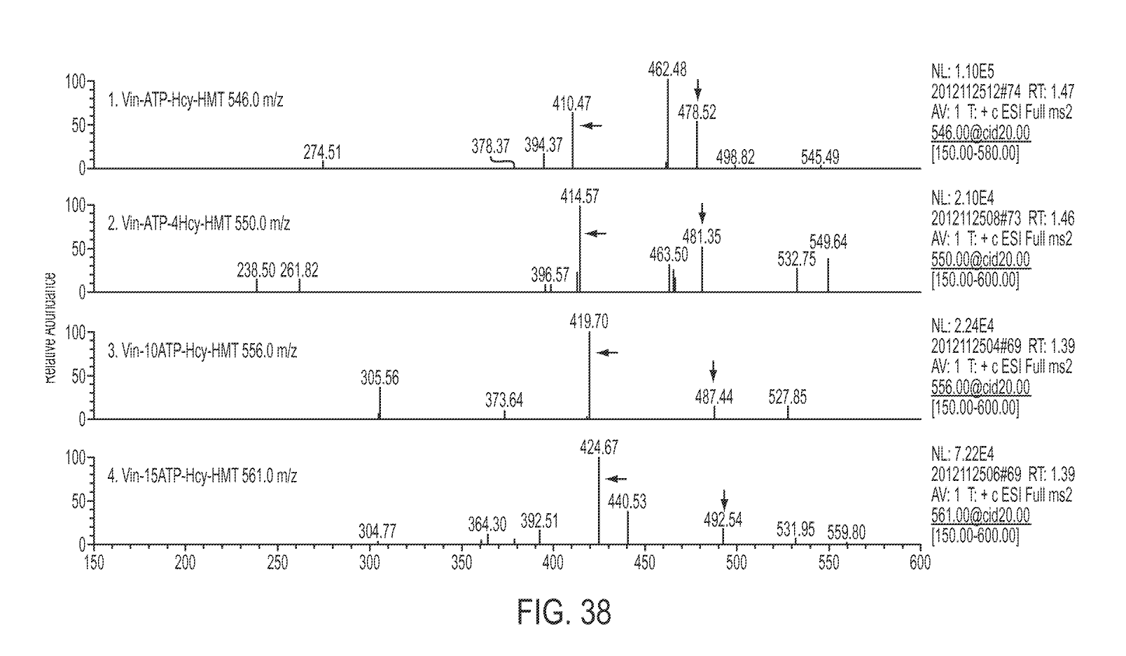

FIG. 38 shows a tandem mass spectra an AdoVin-Hcy adduct. Spectrum 1 is from a reaction between AdoVin and ATP (natural isotopes) in the presence of homocysteine methyltransferase (HMT); the precursor ion is 546.0 m/z. Spectrum 2 is from a reaction between DL-homocysteine-3,3,4,4-D₄ labeled AdoVin and ATP (natural isotopes) in the presence of HMT; the precursor ion is 550.0 m/z. Spectrum 3 is from a reaction between AdoVin and ¹³C₁₀-labeled ATP with HMT in the presence of HMT; the precursor ion is 556.0 m/z. Spectrum 4 is from a reaction between AdoVin and ¹³C₁₀,¹⁵N₅ labeled ATP in the presence of HMT; the precursor ion is 561.0 m/z. The fragmentation patterns and isotope patterns are consistent with the structures of homocysteine-AdoVin adduct.

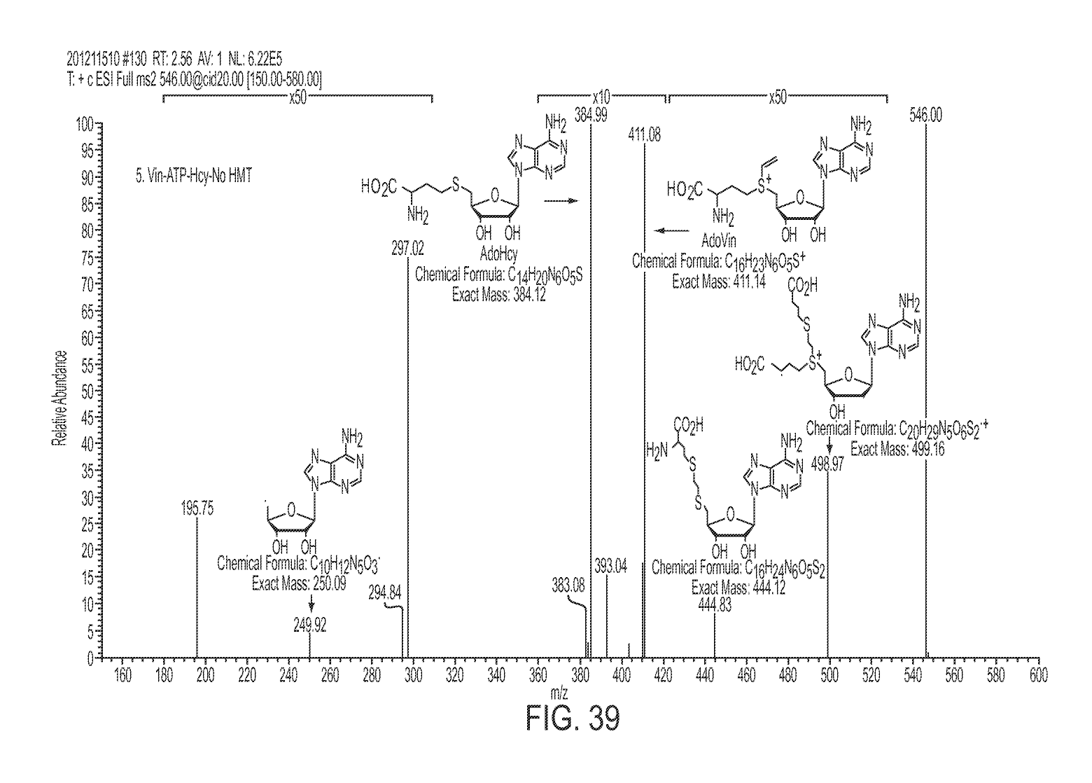

FIG. 39 shows a tandem mass spectrum of an AdoVin-Hcy adduct in the absence of methyltransferase. The precursor ion is 546.0 m/z. The fragmentation patterns and isotope patterns are consistent with the structure of the homocysteine-AdoVin adduct.

### EXAMPLES

The following examples are presented for the purpose of illustration only and are not intended to be limiting.

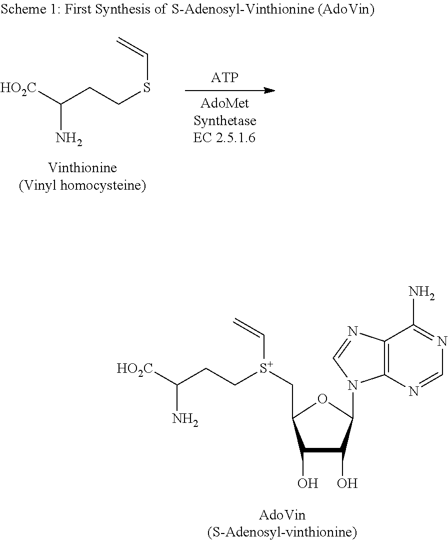

L-S-Vinylhomocysteine (L-vinthionine) was prepared by the method reported by Leopold, W. R. et al. Biochem. Biophys. Res. Commun. 1979, 88, 395 and Jiracek, J. et al. J. Med. Chem. 2006, 49, 3982. L- and DL-S-Vinylhomocysteine are mutagenic, carcinogenic, and hepatotoxic (Leopold 1979, 1982), though DL-S-vinylhomocysteine was shown not to be an inhibitor of human betaine-homocysteine S-methyltransferase (Jiracek 2006). Like the essential amino acid L-methionine, the synthetic amino acid analog L-vinthionine is actively transported into cells where it is enzymatically (methionine S-adenosyl transferase) converted with ATP to the S-demethyl-5-vinyl analog of S-adenosylmethionine (SAM, AdoMet).

S-Adenosyl vinthionine (AdoVin) was synthesized from vinthionine (vinyl homocysteine) and ATP catalyzed by S-adenosyl-methionine synthetase (MAT, EC 2. 5.1.6). The reaction contained 20 mM NH₄HCO₃ (pH 8.0), 25 mM KCl, 10 mM MgCl₂, 5 mM ATP and 1.05 mM vinthionine, and were initiated with 114 μM S-adenosyl-methionine synthetase (MAT, EC 2.5.1.6) and incubated at 37° C. About 300 μM AdoVin was synthesized after 2 h incubation.

HPLC-UV Analysis was conducted as follows. Aliquots of reaction mixture (10 μL) were removed (typically at 0.5, 1 or 2 h) and analyzed by HPLC (monitored at 260 nm). The chromatography was performed on a reverse-phase column (Apollo, C₁₈, 5μ, ID 4.6 mm, length 150 mm) using 0.1% aqueous TFA (mobile phase A) and 0.1% TFA in acetonitrile (mobile phase B) at a flow rate of 1 mL/min. The gradient program was initiated with 2% mobile phase B, followed by a linear increase to 10% mobile phase B over 8 min, then a return to 2% mobile phase B over 1 min, and finally a hold at 2% mobile phase B over 3 min.

HPLC-UV-MS Analysis was conducted as follows. An aliquot of reaction mixture (20 μL) at 2 h incubation was injected into LC-UV-MS (Ion Trap MS). The chromatography was performed on a reverse-phase column (Apollo, C₁₈, 5μ, ID 4.6 mm, length 150 mm) using 0.1% aqueous formic acid (mobile phase A) and 0.1% formic acid in acetonitrile (mobile phase B) at a flow rate of 1 mL/min. The gradient program was the same with HPLC-UV analysis described above.

FIG. 1 shows an HPLC chromatogram (monitored at 260 nm) of AdoVin synthesis with (top trace) or without (middle trace) MAT and S-adenosyl methionine (AdoMet) synthesis (bottom trace).

FIG. 2 shows an extracted ion chromatogram (XIC) of 411 m/z (AdoVin) and 206.0 m/z (doubly charged AdoVin).

FIG. 3 shows a mass spectrum at 3.75 min (411.07 m/z is for AdoVin).

FIG. 4 shows a MS/MS spectrum of the precursor ion of 411.07 m/z (AdoVin).

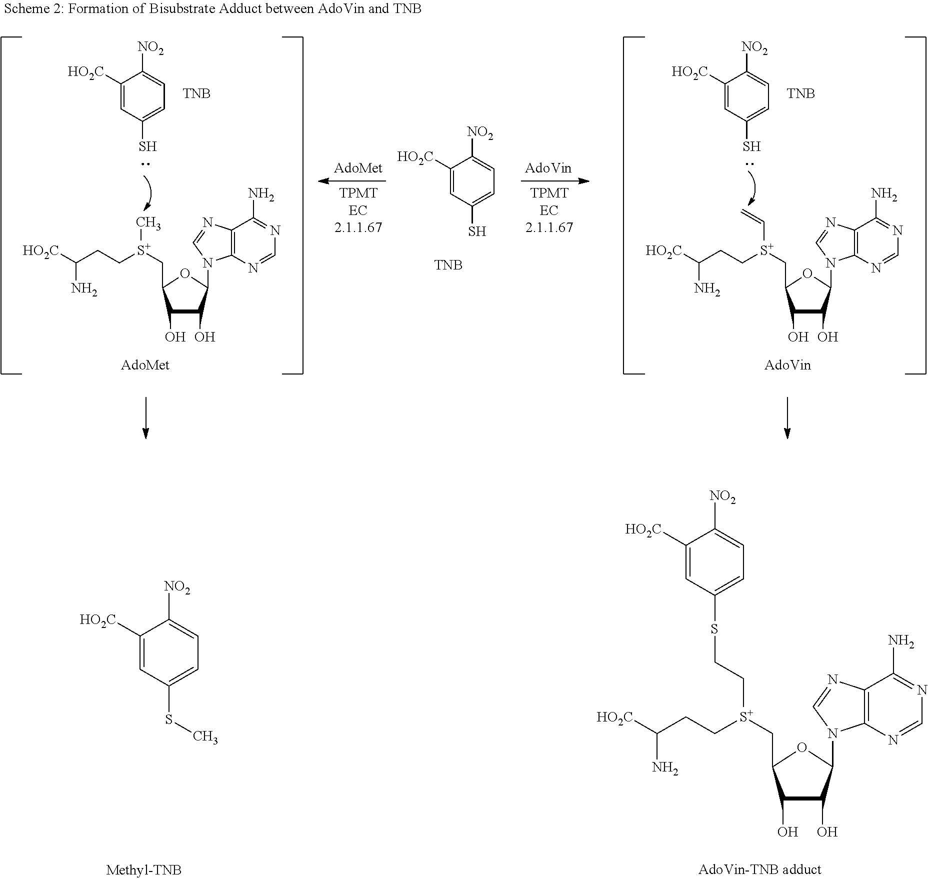

Methylation of 5-Thio-2-Nitrobenzoic Acid (TNB) (left) and formation of bisubstrate-adduct between AdoVin and TNB (right) that are catalyzed by thiopurine methyltransferase (TPMT)

The reaction contained 50 mM NH₄HCO₃ (pH 8.0), 10 mM KCl, 4 mM MgCl₂, 2 mM ATP, 600 μM vinthionine (vinyl homocysteine), 480 μM TNB, 83 μM thiopurine methyltransferase (TPMT, EC 2.1.1.67), and were initiated with 120 μM 5-adenosyl-methionine synthetase (MAT, EC 2.5.1.6) and incubated at 37° C. About 6.54 μM of AdoVin-TNB adduct formed after 1 h incubation (determined by UV absorption of the adenine chromophore with ε260 nm=15,400 M⁻¹ cm⁻¹).

The reaction was monitored by taking aliquots (10 μL) of reactions (typically 1, 3 or 6 h) and injecting and analyzing by HPLC (monitored at 260 nm and 350 nm, based on the absorbance for AdoMet and substituted TNB). The chromatography was performed on reverse phase column (Apollo, C₁₈, 5μ, 4.6 mm×150 mm) using 0.1% aqueous TFA (mobile phase A) and 0.1% TFA in acetonitrile (mobile phase B) at a flow rate of 1 mL/min. The gradient program was initiated with 2% mobile phase B for 5 min, followed by a linear increase to 60% mobile phase B over 22 min, then a return to 2% mobile phase B over 1 min, and finally a hold at 2% mobile phase B over 3 min. Formic acid was used in HPLC-MS assay, instead of TFA.

In the presence of 5-thio-2-nitrobenzoic acid and AdoVin, thiopurine methyltransferase catalyzes formation of a bi-substrate adduct. There are two possible adduct structures. Mass spectral analysis could not distinguish if one, the other, or both are formed.

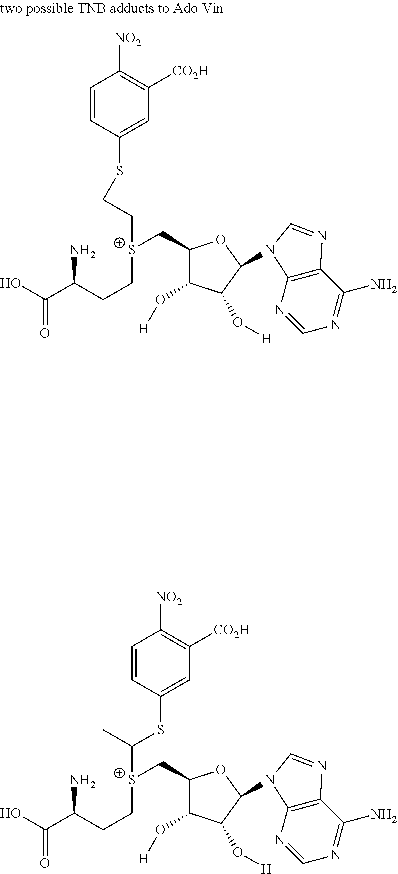 [SMILES: Nc1ncnc2c1ncn2[C@@H]1O[C@H](C[S+](CCSc2ccc([N+](=O)[O-])c(C(=O)O)c2)CC[C@H](N)C(=O)O)[C@@H](O)[C@H]1O ; CC(Sc1ccc([N+](=O)[O-])c(C(=O)O)c1)[S+](CC[C@H](N)C(=O)O)C[C@H]1O[C@@H](n2cnc3c(N)ncnc32)[C@H](O)[C@@H]1O]

Regardless of whether the bi-substrate adduct is the 1,2-ethane disulfide or the acetaldehyde dithioacetal adduct (the latter likely to be a highly reactive intermediate that can covalently modify and inactivate the methyltransferase enzyme), the bi-substrate adduct binds to thiopurine methyltransferase (TPMT, EC 2.1.1.67) with high affinity (with no dissociation observed using filtration assay) and inactivates the enzyme analogously to related high-affinity, transition state-like, competitive enzyme inhibitors. Jiracek, J. et al. J. Med. Chem. 2006, 49, 3982; Nikodejevic, B. et al. J. Pharmacol. Exp. Ther. 1970, 174, 83; Anderson, G. L.; et al. J. Med. Chem. 1981, 24, 1271; Korolev, S. et al. Nat. Struct. Biol. 2002, 9, 27; Nikodejevic, B. et al. J. Pharmacol. Exp. Ther. 1970, 174, 83.

Analogously, bi-substrate adduct(s) of AdoVin and homocysteine inactivate homocysteine S-methyltransferase (HMT, EC 2.1.1.10) enzyme-mediated adduct formation mechanism.

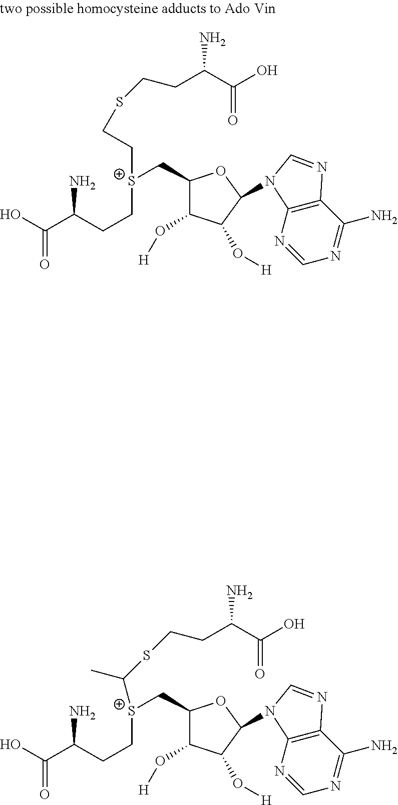 [SMILES: Nc1ncnc2c1ncn2[C@@H]1O[C@H](C[S+](CCSCC[C@H](N)C(=O)O)CC[C@H](N)C(=O)O)[C@@H](O)[C@H]1O ; CC(SCC[C@H](N)C(=O)O)[S+](CC[C@H](N)C(=O)O)C[C@H]1O[C@@H](n2cnc3c(N)ncnc32)[C@H](O)[C@@H]1O]

AdoVin and other analogs of endogenous AdoMet inhibit cysteine methylation of zinc-finger motifs of proteins that regulate eukaryotic pathways by the mechanism of adduct formation with protein cysteine methyltransferases, tight-binding of the bi-substrate ligand, and inhibition of methyltransferase activity (Zhang 2012).

As noted by Zhang 2012, cysteine methylation disrupts ubiquitin-chain sensing in NF-κB activation. NF-κB is crucial for innate immune defense against microbial infection, and inhibition of NF-κB signaling has been observed with various bacterial infections, including by methylation. Accordingly, the present disclosure includes the use of AdoVin and its analogs as antibacterials.

AdoVin may inactivate DNA methyltransferases that are responsible for regulation of DNA translation, particularly DNA methyl transferases with cysteine residues at or near catalytic sites, by the general mechanism of tight-binding bi-substrate adduct inhibition of enzyme activity as described above (Siddique 2011).

HPLC-UV analysis was conducted as follows. Aliquots (10 μL) of reactions (typically 1, 3 or 6 h) were injected and analyzed by HPLC (monitored at 260 nm and 350 nm). The chromatography was performed on reverse phase column (Apollo, C₁₈, 5μ, 4.6 mm×150 mm) using 0.1% aqueous TFA (mobile phase A) and 0.1% TFA in acetonitrile (mobile phase B) at a flow rate of 1 mL/min. The gradient program was initiated with 2% mobile phase B for 5 min, followed by a linear increase to 60% mobile phase B over 22 min, then a return to 2% mobile phase B over 1 min, and finally a hold at 2% mobile phase B over 3 min.

HPLC-Mass Spectrometry analysis was conducted as follows. An aliquot (20 μL) of reaction at 1 h incubation were injected into LC-UV-MS (Ion Trap MS). The chromatography was performed using 0.1% aqueous formic acid (mobile phase A) and 0.1% formic acid in acetonitrile (mobile phase B) at a flow rate of 1 mL/min. The gradient program was the same with HPLC-UV analysis described above.

FIG. 5 shows an HPLC chromatogram (monitored at 350 nm) of the reaction between AdoVin and TNB catalyzed by TPMT at 1, 3 and 6 h.

FIG. 6 shows an HPLC chromatogram (monitored at 350 nm) of the reaction between AdoVin and TNB with (top trace) and without (middle trace) TPMT and without AdoMet synthetase (MAT, negative control) at 1 h.

FIG. 7 shows a UV spectra of adenosine, methyl-TNB and AdoVin-TNB adduct.

FIG. 8 shows a calculation of extinction coefficient of the AdoVin-TNB Adduct.

FIG. 9 shows the calculated mass for TNB, AdoVin and their adduct.

FIG. 10 shows an extracted ion chromatogram (XIC) of 411.0 m/z (AdoVin), and 610.0 m/z (AdoVin-TNB adduct) m/z for reactions between AdoVin and TNB with (top) or without TPMT (bottom).

FIG. 11 shows a mass spectrum at 8.55 min of (610.0 m/z is for the AdoVin-TNB adduct).

FIG. 12 shows an MS/MS spectrum of the precursor ion of 610.0 m/z (for the AdoVin-TNB adduct).

### Scheme 3: Inactivation of AdoMet-Binding Proteins by AdoVin

AdoVin is generated in situ from vinthionine and ATP using methionine adenosyltransferases (MAT) as a catalyst. AdoVin is allowed to incubate with AdoMet-binding proteins. The competitive binding of AdoVin against AdoMet is sufficient to inactivate AdoMet-binding proteins.

### Scheme 4: Inactivation of AdoMet-Binding Proteins by AdoVin

AdoVin is supplied as a purified compound. AdoVin is incubated with AdoMet-binding proteins over a period of time to allow the residues at or near the AdoMet binding sites in the proteins to chemically react with AdoVin. This results in modification and inactivation of the proteins.

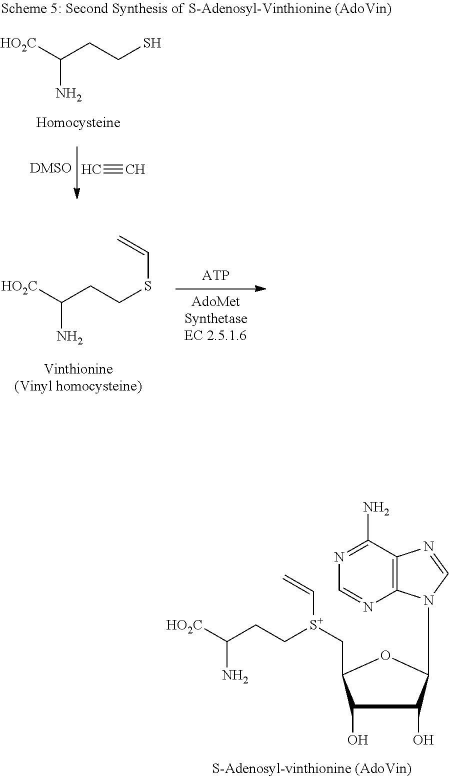

Reaction complexes were analyzed by reversed-phase HPLC (Apollo 5 μm, C18 150×4.6 mm column) using a Agilent 1100 HPLC system. Mass spectral data were acquired on a Thermo LCQ Deca Ion trap mass spectrometer attached to an Agilent 1200 HPLC, and processed by using Xcalibar Data System 2.0.7 (Thermo Fisher Scientific Inc. Waltham, Mass.). Graphics were constructed by using the Kaleidagraph software package 4.1 (Synergy Software, Reading, Pa.).

Recombinant histidine-tagged human thiopurine methyltransferase (TPMT, EC 2.1.1.67) and S-adenosylhomocysteine nucleosidase (MTAN, EC 3.2.2.9) were purified as described. See Cannon, L. M. et al. Anal. Biochem. 2002, 308, 35/-363; Lee, J. E. et al. Acta Crystallogr. D. Biol. Crystallogr. 2001, 57, 150-152. The recombinant E. coli homocysteine S-methyltransferase (HMT, EC 2.1.1.10) was a gift from Steven G Clark (UCLA Department of Chemistry and Biochemistry, Los Angeles, Calif.), and purified as described. See Vinci, C. R.; Clarke, S. G. J. Biol. Chem. 2010, 285(27), 20526-20531; Vinci, C. R.; Clarke, S. G. J. Biol. Chem. 2007, 282(12), 8604-8612.

The concentration of 3-carboxy-4-nitro-benzenethiol (TNB) was determined using ε412 nm=13,600 M⁻¹ cm⁻¹ and the concentration of S-adenosyl vinthionine (AdoVin)was determined using ε260 nm=15,400 M⁻¹ cm⁻¹ based on the values for AdoMet and AdoHcy. Cannon, L. M. et al. Anal. Biochem. 2002, 308, 358-363.

To a 250 mL, three-necked, round-bottom flask over a dry-ice/ethanol bath, homocysteine (1.01 g, 7.47 mmol, racemic mixture) was added. The sealed system was evacuated and then purged with anhydrous nitrogen several times Anhydrous ammonia was then condensed into the round-bottom flask until the volume of liquid ammonia was approximately 50 mL. Small pieces of sodium metal were added slowly with stirring until the solution remained blue for ten minutes. NH⁴Cl was added slowly until the blue color faded, and the solution was allowed to warm to room temperature. After all ammonia was evaporated, the flask was cooled to 0° C. and 15 mL of anhydrous DMSO was added to dissolve the residue. Dried acetylene gas was then bubbled though the solution for 4 hours, during which the flask was allowed to return room temperature. The syrup was then neutralized to pH near 7 with 1N HCl. The mixture was cooled to −20° C. overnight to allow complete participate before the mixture was filtered and washed with cold ethanol. The light-yellow powder residue was dried under vacuum. This material was used in next step without further purification. ¹H NMR (D₂O/K₂CO₃, 400 MHz): δ 6.33 (dd, 1H, J=16.9 Hz, 10.3 Hz), 5.24 (d, 1H, J=10.3 Hz), 5.16 (d, 1H, J=16.9 Hz), 3.74 (t, 1H, J=6.2 Hz), 2.77 (t, 2H, J=7.3 Hz), 2.10 (m, 2H); ¹³C NMR (D2O, 100 MHz): δ 174.45, 130.68, 112.70, 53.94, 30.17, 26.42.

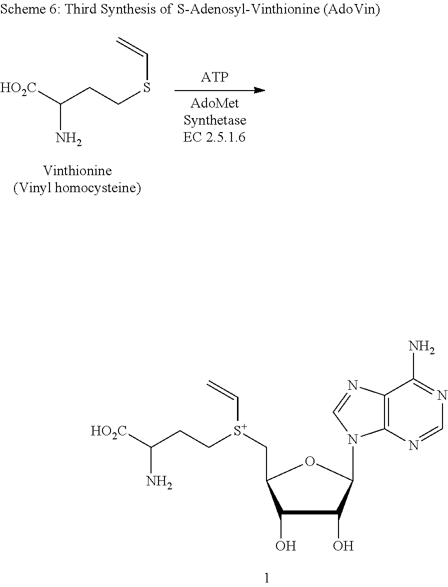

AdoVin was prepared, catalyzed by AdoMet synthetase (methionine adenosyltransferase, MAT, EC 2.5.1.6). The concentration of S-adenosyl vinthionine (AdoVin) was determined using ε260 nm=15,400 M⁻¹ cm⁻¹. The reaction was monitored by HPLC-MS/MS. The reaction contained 20 mM NH₄HCO₃ (pH 8.0), 25 mM KCl, 10 mM MgCl₂, 5 mM ATP and 1.05 mM vinthionine, and were initiated with 114 μM S-adenosyl-methionine synthetase (MAT, EC 2.5.1.6) and incubated at 37° C. About 300 μM AdoVin was synthesized after 2 h incubation. Aliquots of reaction mixture (10 μL) were removed at 0.5, 1 and 2 hours and analyzed by reverse-phase HPLC (Apollo, C18, 5μ, ID 4.6 mm, length 150 mm, monitored at 260 nm, using 0.1% aqueous TFA (mobile phase A) and 0.1% TFA in acetonitrile (mobile phase B) at a flow rate of 1 mL/min). The gradient program was initiated with 2% mobile phase B, followed by a linear increase to 10% mobile phase B over 8 min, then a return to 2% mobile phase B over 1 min, and finally a hold at 2% mobile phase B over 3 min. Formic acid was used in HPLC-MS assay, instead of TFA. Monitoring of this reaction is shown in FIG. 27.

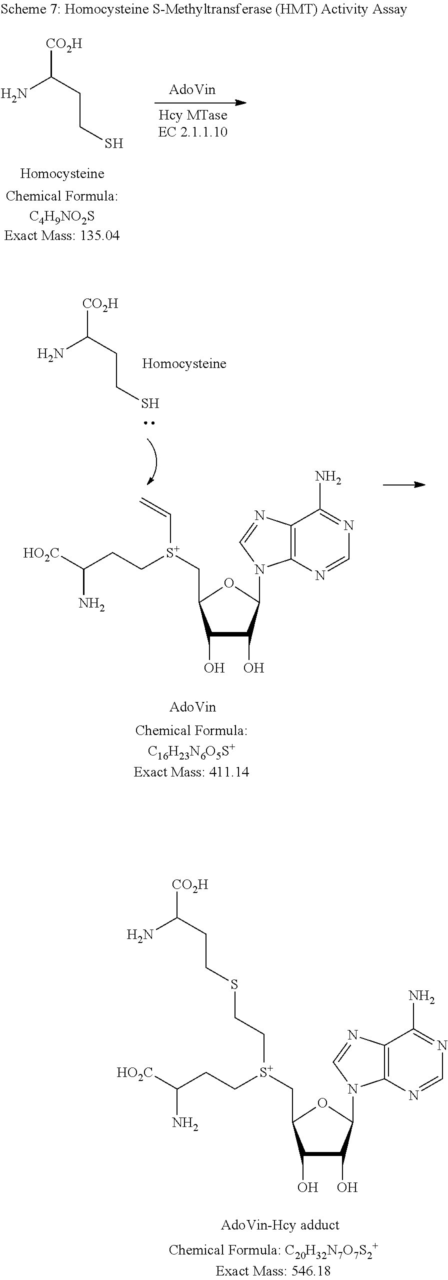

Scheme 4 shows the formation of bisubstrate adduct between AdoVin and homocysteine catalyzed by Homocysteine S-Methyltransferase (HMT, EC 2.1.1.10).

The reaction contained 50 mM NH₄HCO₃ (pH 7.5), 5 mM KCl, 2 mM MgCl₂, 1 mM ATP, 300 μM vinthionine (vinyl homocysteine), 375 μM homocysteine, 123 μM homocysteine S-methyltransferase (HMT, EC 2.1.1.10), and was initiated with 65.2 μM S-adenosyl-methionine synthetase (MAT, EC 2.5.1.6) and incubated at 37° C. Aliquots (10 μL) of reactions (typically 4 and 8 h) were injected and analyzed by HPLC (monitored at 260 nm, based on the absorbance for AdoMet). The chromatography was performed on reverse phase column (Apollo, C₁₈, 5μ, 4.6 mm×150 mm) using 0.1% aqueous formic acid (mobile phase A) and 0.1% formic acid in acetonitrile (mobile phase B) at a flow rate of 1 mL/min. The gradient program was initiated with 100% mobile phase A for 5 min, followed by a linear increase to 20% mobile phase B over 15 min, then a return to 100% mobile phase A over 1 min, and finally a hold at 100% mobile phase A over 4 min. Isotopic reagents, adenosine-¹³C₁₀,¹⁵N₅ 5′-triphosphate sodium salt solution (Sigma-Aldrich, catalog number 645702), adenosine-¹³C₁₀ 5′-triphosphate sodium salt solution (Sigma-Aldrich, catalog number 710695) and DL-homocystine-3,3,3′,3′,4,4,4′,4′-d₈ (Sigma-Aldrich, catalog number 724955) were used to verify the adducts.

Those skilled in the art would readily appreciate that all parameters and configurations described herein are meant to be exemplary and that actual parameters and configurations will depend upon the specific application for which the systems and methods of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that the invention may be practiced otherwise than as specifically described. Accordingly, those skilled in the art would recognize that the use of an electrochemical device in the examples should not be limited as such. The present invention is directed to each individual feature, system, or method described herein. In addition, any combination of two or more such features, systems or methods, if such features, systems or methods are not mutually inconsistent, is included within the scope of the present invention.

### REFERENCES

The following documents are incorporated herein by reference in their entireties.
1. Leopold, W. R.; Miller, J. A.; Miller, E. C. Cancer Res. 1982, 42, 4364.
2. Kenton, N. T. “Design and synthesis of methionine analogues for the enzymatic synthesis of S-adenosyl-methionine analogues: tools for the analysis of methyltransferase substrate specificity” (2012), Chemistry Master's Theses, Paper 22, available at http://hdl.handle.net/2047/d20002527 (last visited Mar. 29, 2013).
3. Leopold, W. R.; Miller, J. A.; Miller, E. C. Biochem. Biophys. Res. Commun. 1979, 88, 395.
4. Jiracek, J.; Collinsova, M.; Rosenberg, I.; Budesinsky, M.; Protivinska, E.; Netusilova, H.; Garrow, T. A. J. Med. Chem. 2006, 49, 3982.
5. Goldberg, B.; Yarlett, N.; Rattendi, D.; Lloyd, D.; Bacchi, C. J. J. Eukaryot. Microbiol. 1997, 44, 345.
6. Goldberg, B.; Yarlett, N.; Sufrin, J.; Lloyd, D.; Bacchi, C. J. FASEB J. 1997, 11, 256.
7. Lawrence, F.; Robert-Gero, M. J. Eukaryot. Microbiol. 1993, 40, 581.
8. Avila, J. L.; Polegre, M. A. Mol. Biochem. Parasitol. 1993, 58, 123.
9. Nikodejevic, B.; Senoh, S.; Daly, J. W.; Creveling, C. R. J. Pharmacol. Exp. Ther. 1970, 174, 83.
10. Anderson, G. L.; Bussolotti, D. L.; Coward, J. K. J. Med. Chem. 1981, 24, 1271.
11. Korolev, S.; Ikeguchi, Y.; Skarina, T.; Beasley, S.; Arrowsmith, C.; Edwards, A.; Joachimiak, A.; Pegg, A. E.; Savchenko, A. Nat. Struct. Biol. 2002, 9, 27.
12. Zhang, L.; Ding, X.; Cui, J.; Xu, H.; Chen, J.; Gong, Y.-N.; Hu, L.; Zhou, Y.; Ge, J.; Lu, Q.; Liu, L.; Chen, S.; Shao, F. Nature 2012, 481, 204.
13. Siddique, A. N.; Jurkowska, R. Z.; Jurkowski, T. P.; Jeltsch, A. FEBS J. 2011, 278, 2055.
14. Markham, G. D S-Adenosylmethionine. In: Encyclopedia of Life Sciences (ELS), John Wiley & Sons, Ltd: Chichester, April 2010, pp. 1-8.
15. Fontecave, M. et al. Trends Biochem. Sci. 2004, 29(5), 243-249.
16. Markham, G. D. et al. Biochemistry 2002, 41(24),7636-7646.
17. Dowling, D. P. et al. Biochim. Biophys. Acta 2012, 1824(11), 1178-1195.
18. Lanz, N. D.; Booker, S. J. Biochim. Biophys. Acta 2012, 1824(11), 1196-212.
19. Bandarian, V. Biochim. Biophys. Acta. 2012, 1824(11), 1245-1253.
20. Zhang, Q. et al. Acc. Chem. Res. 2012, 45(4), 555-564.
21. Lin, H. Bioorg. Chem. 2011, 39(5-6), 161-170.
22. Vey, J. L.; Drennan, C. L. Chem. Rev. 2011, 1144), 2487-2506.
23. Li, Y. et al. Chem. Biol. Interact. 2005, 152(2-3), 151-163.
24. Cotton, N. J. et al. J. Biol. Chem. 2004, 279(22), 23710-23718.
25. Wang, S. C.; Frey, P. A. Trends Biochem. Sci. 2007, 32(3), 101-110; Erratum in: Trends Biochem. Sci. 2007, 32(5), 209.
26. Sohn, Y. W. et al. Carcinogenesis 1996, 17(6), 1361-1364.
27. Kim, H. J. et al. Anal. Biochem. 1992, 207(1), 68-72.
28. Koechel, D. A. et al. Fundam. Appl. Toxicol. 1991, 17(1), 17-33.
29. Leopold, W. R. et al. Cancer Res. 1982, 42(11), 4364-4374.
30. Warner, D. R.; Hoffman, J. L. Biochemistry 1996, 35(14), 4480-4484.
31. Zhao, G.; Zhou, Z. S. Bioorg. Med. Chem. Lett. 2001, 11(17), 2331-2335.
32. Horowitz, S. et al. J. Biol. Chem. 2011, 286(21), 18658-18663.
33. Schubert, H. L. et al. Trends Biochem. Sci. 2003, 28(6), 329-33.
34. Cochrane, J. C.; Strobel, S. A. RNA 2008, 14(6), 993-1002.
35. Boyapati, V. K. et al. RNA 2012, 18(6), 1230-1243.
36. Edwards, A. L. et al. RNA 2010, 16(11), 2144-2155.
37. Ottink, O. M. et al. Anal. Biochem. 2010, 396(2), 280-283.
38. Baykov, A. A. et al. ACS Chem. Biol. 2011, 6(11), 1156-1163.
39. Hnizda, A. et al. Biochemistry 2010, 49(49), 10526-10534.
40. Pey, A. L. et al. Biochem. J. 2013, 449(1), 109-121
41. Le Calvez, P. B. et al. J. Enzyme Inhib. Med. Chem. 2009, 24(6), 1291-1318.
42. Korolev, S. et al. Nat. Struct. Biol. 2002, 9(1), 27-31.
43. Wall, M. et al. J. Med. Chem. 1999, 42(18), 3421-3424.
44. Burns, M. R.; Coward, J. K. Bioorg. Med. Chem. 1996, 4(9), 1455-1470.
45. Lakanen, J. R. J. Med. Chem. 1995, 38(14), 2714-27.
46. Pegg, A. E. et al. Int. J. Biochem. Cell. Biol. 1995, 27(5), 425-442.
47. Anderson, G. L. et al. J. Med. Chem. 1981, 24(11), 1271-1277.
48. Tang, K. C. et al. J. Med. Chem. 1981, 24(11), 1277-1284.

1. A compound of Formula I: 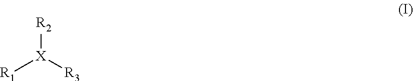
or a salt, hydrate or solvate thereof;
wherein
X is C, CR, N, NR, NOR, P, PR, POR, P(R)R′, P(OR)R′, P(OR)OR′, P(O), S, SR, SOR, S(R)R′, S(OR)R′, S(OR)OR′, S(O), S(R)(R′)R″, S(R)(R′)OR″, S(R)(OR′)OR″, S(OR)(OR′)OR″, S(O)R, S(O)R, Se, SeR, SeOR, Se(R)R′, Se(OR)R′, Se(OR)OR′, Se(O), Se(R)(R′)R″, Se(R)(R′)OR″, Se(R)(OR′)OR″, Se(OR)(OR′)OR″, Se(O)R, Se(O)OR, SS, SeSe, SSe or SeS;
R₁ is —R, a substituted or unsubstituted amino acid, C₁₋₁₂amino alcohol, C₁₋₁₂carboxylic acid, —OR, ═O, or R₁ and X taken together are: 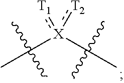
wherein T₁ and T₂ are each independently —OR or ═O;
R₂ is an electrophile;
wherein X and R₂ taken together can form a 3-to-10-membered ring;
R₃ is a nucleotide, nucleoside or a derivative thereof; and
R, R′ and R″ are each independently H or a substituted or unsubstituted C₁₋₁₀alkyl, C₂₋₁₀alkenyl, C₂₋₁₀alkynyl, C₆₋₁₀aryl, C₃₋₈cycloalkyl, C₄₋₆heterocyclyl or C₅₋₆heteroaryl.

2. The compound of claim 1, wherein R₃ is selected from the group consisting of adenosine, deoxyadenosine, guanosine, deoxyguanosine, 5-methyluridine, thymidine, uridine, deoxyuridine, cytidine, deoxycytidine, formycin, aristeromycin, didanosine, inosine, acyclovir, deoxyinosine, abacavir, N4-acetylcytidine, allopurinol riboside, 2′-O-allyladenosine, 3′-O-allyladenosine, 3′-O-allylcytidine, 2′-O-allylcytidine, 2′-O-allylguanosine, 3′-O-allylguanosine, 2′-O-allyluridine, 3′-O-allyluridine, bromodeoxyuridine, cytarabine, azacitidine, decitabine, pseudouridine, S-adenosyl-L-homocysteine, pentostatin, regadenoson, telbivudine, 8-oxo-2′-deoxyguanosine, CGS-21680, floxuridine, 5-methyluridine, dihydrouridine, nelarabine, xanthosine, maribavir, 8-hydroxyguanosine, N4-chloroacetylcytosine arabinoside, sapacitabine, orotidine, queuosine, lysidine, fialuridine, CP-532,903, cordycepin, tezacitabine, dexelvucitabine, N6-cyclopentyladenosine, iododeoxyuridine, PSI-6130, 5,6-dichloro-1-beta-D-ribofuranosylbenzimidazole, S-adenosylmethioninamine, FV-100 and 5-ethynyl-2′-deoxyuridine, 9-β-D-allopyranosyl-9H-Purin-6-amine, (S)-9-(2,3-dihydroxypropyl)adenine (DHPA), D-eritadenine, 9-(2-bromo-4-hydroxy-3-hydroxymethyl-2-butenyl)adenine, 1-(6-amino-9H-purin-9-yl)-1,5-dideoxy-D-Arabinitol, S-8-aza-adenosylmethionine (8-aza-SAM), S-2-aminopurinylmethionine (2AP-SAM), S-2,6-diaminopurinylmethionine (DAPSAM), and 2,6-diaminopurine (DAP).

3. The compound of any preceding claim, wherein the electrophile is a substituted or unsubstituted C₂₋₁₀alkene, C₂₋₁₀alkyne, C₂₋₁₀ketone, C₁₋₁₀aldehyde or C₁₋₁₀alkyl halide.

4. The compound of any preceding claim, wherein the nucleotide, nucleoside or derivative thereof is bound through the pentose ring, hexose ring, or through the open-chain.

5. The compound of any preceding claim, wherein the nucleotide, nucleoside or derivative thereof is bound through the 5′ position of the pentose ring.

6. The compound of any preceding claim, wherein the compound is a compound of Formula II or Formula III: 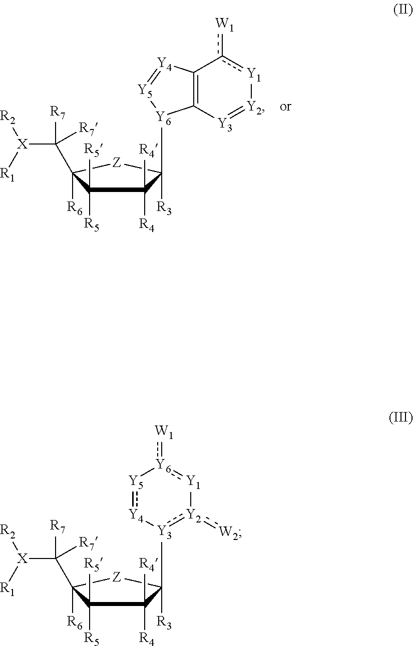
wherein
W₁ and W₂ are each independently selected from R, O, OR, OC(O)R, OC(O)OR, OC(O)N(R)R′, N(R)R′, NC(O)R, NC(O)OR, NC(O)N(R)R′, P, PR, POR, P(R)R′, P(OR)R′, P(OR)OR′, P(O), P(R)(R′)R″, P(R)(R′)OR″, P(R)(OR′)OR″, P(OR)(OR′)OR″, P(O)R, P(O)OR, P(R)(R′)(R″)R′″, P(R)(R′)(R″)OR′″, P(R)(R′)(OR″)OR′″, P(R)(OR′)(OR″)OR′″, P(OR)(OR′)(OR″)OR′″, P(O)(R)R′, P(O)(R)OR′, P(O)(OR)OR″, PO₂, S, SR, SOR, S(R)R′, S(OR)R′, S(OR)OR′, S(O), S(R)(R′)R″, S(R)(R′)OR″, S(R)(OR′)OR″, S(OR)(OR′)OR″, S(O)R, S(O)R, S(R)(R′)(R″)R′″, S(R)(R′)(R″)OR′″, S(R)(R′)(OR″)OR′″, S(R)(OR′)(OR″)OR′″, S(OR)(OR′)(OR″)OR′, S(O)(R)R′, S(O)(R)OR′, S(O)(OR)OR″, SO₂, S(R)(R′)(R″)(R′″)R″″, S(R)(R′)(R″)(R′″)OR″″, S(R)(R′)(R″)(OR′″)OR′, S(R)(R′)(OR″)(OR′″)OR″″, S(R)(OR′)(OR″)(OR′″)OR″″, S(OR)(OR′)(OR″)(OR′″)OR″″, S(O)(R)(R′)R″, S(O)(R)(R′)OR″, S(O)(R)(OR′)OR″, S(O)(OR)(OR′)OR″, SO₂R, SO₂OR, Se, SeR, SeOR, Se(R)R′, Se(OR)R′, Se(OR)OR′, Se(O), Se(R)(R′)R″, Se(R)(R′)OR″, Se(R)(OR′)OR″, Se(OR)(OR′)OR″, Se(O)R, Se(O)OR, Se(R)(R′)(R″)R′″, Se(R)(R′)(R″)OR′″, Se(R)(R′)(OR″)OR′″, Se(R)(OR′)(OR″)OR′″, Se(OR)(OR′)(OR″)OR′, Se(RO)(R)R′, Se(RO)(R)OR′, Se(O)(OR)OR″, SeO₂, Se(R)(R′)(R″)(R′″)R″″, Se(R)(R′)(R″)(R′″)OR″″, Se(R)(R′)(R″)(OR′″)OR′, Se(R)(R′)(OR″)(OR′″)OR′, Se(R)(OR′)(OR″)(OR′″)OR″″, Se(OR)(OR′)(OR″)(OR′″)OR″″, Se(O)(R)(R′)R″, Se(O)(R)(R′)OR″, Se(O)(R)(OR′)OR″, Se(O)(OR)(OR′)OR″, SeO₂R, SeO₂OR, SSR, SeSeR, SSeR, or SeSR;
Y₁, Y₂, Y₃, Y₄, Y₅ and Y₆ are each independently selected from C, CR, CC(O)R, CC(O)OR, CC(O)N(R)R′, CN(R)R′, N, NR, NC(O)R, or NC(O)OR;
Z is R, O, N(R)R′, S, S(O), or SO₂;
R₃, R₄, R_{4′}, R₅, R_{5′}, R₆, R₇ and R_{7′} are each independently selected from —R, —OR, —N(R)R′, —C(O)R, —C(O)OR, —C(O)N(R)R′, a substituted or unsubstituted amino acid, C₁₋₁₂amino alcohol, or C₁₋₁₂carboxylic acid; and
R, R′ R″, R′ and R″″ are each independently H or a substituted or unsubstituted C₁₋₁₀alkyl, C₂₋₁₀alkenyl, C₂₋₁₀alkynyl, C₆₋₁₀aryl, C₃₋₈cycloalkyl, C₄₋₆heterocyclyl or C₅₋₆heteroaryl.

7. The compound of any one of claims 1 to 5, wherein the compound is a compound of Formula V, Formula VI or Formula VII: 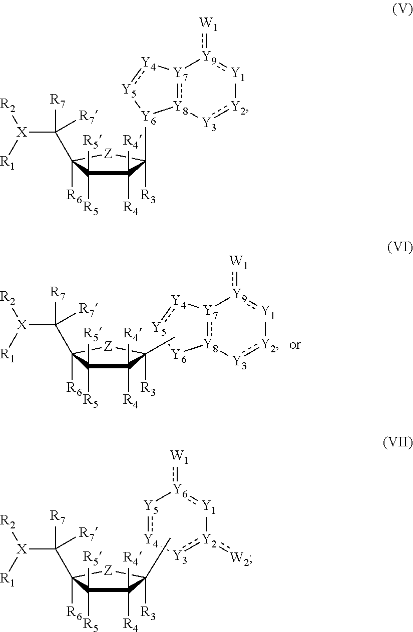
wherein
W₁ and W₂ are each independently selected from R, O, OR, OC(O)R, OC(O)OR, OC(O)N(R)R′, N(R)R′, NC(O)R, NC(O)OR, NC(O)N(R)R′, P, PR, POR, P(R)R′, P(OR)R′, P(OR)OR′, P(O), P(R)(R′)R″, P(R)(R′)OR″, P(R)(OR′)OR″, P(OR)(OR′)OR″, P(O)R, P(O)OR, P(R)(R′)(R″)R′″, P(R)(R′)(R″)OR′″, P(R)(R′)(OR″)OR′, P(R)(OR′)(OR″)OR′″, P(OR)(OR′)(OR″)OR′″, P(O)(R)R′, P(O)(R)OR′, P(O)(OR)OR″, PO₂, S, SR, SOR, S(R)R′, S(OR)R′, S(OR)OR′, S(O), S(R)(R′)R″, S(R)(R′)OR″, S(R)(OR′)OR″, S(OR)(OR′)OR″, S(O)R, S(O)OR, S(R)(R′)(R″)R′″, S(R)(R′)(R″)OR′″, S(R)(R′)(OR″)OR′″, S(R)(OR′)(OR″)OR′″, S(OR)(OR′)(OR″)OR′, S(O)(R)R′, S(O)(R)OR′, S(O)(OR)OR″, SO₂, S(R)(R′)(R″)(R′″)R″″, S(R)(R′)(R″)(R′″)OR″″, S(R)(R′)(R″)(OR′″)OR′, S(R)(R′)(OR″)(OR′″)OR″″, S(R)(OR′)(OR″)(OR′″)OR″″, S(OR)(OR′)(OR″)(OR′″)OR″″, S(O)(R)(R′)R″, S(O)(R)(R′)OR″, S(O)(R)(OR′)OR″, S(O)(OR)(OR′)OR″, SO₂R, SO₂OR, Se, SeR, SeOR, Se(R)R′, Se(OR)R′, Se(OR)OR′, Se(O), Se(R)(R′)R″, Se(R)(R′)OR″, Se(R)(OR′)OR″, Se(OR)(OR′)OR″, Se(O)R, Se(O)OR, Se(R)(R′)(R″)R′″, Se(R)(R′)(R″)OR′″, Se(R)(R′)(OR″)OR′″, Se(R)(OR′)(OR″)OR′″, Se(OR)(OR′)(OR″)OR′″, Se(RO)(R)R′, Se(RO)(R)OR′, Se(O)(OR)OR″, SeO₂, Se(R)(R′)(R″)(R′″)R″″, Se(R)(R′)(R″)(R′″)OR″″, Se(R)(R′)(R″)(OR′″)OR′, Se(R)(R′)(OR″)(OR′″)OR′, Se(R)(OR′)(OR″)(OR′″)OR″″, Se(OR)(OR′)(OR″)(OR′″)OR″″, Se(O)(R)(R′)R″, Se(O)(R)(R′)OR″, Se(O)(R)(OR′)OR″, Se(O)(OR)(OR′)OR″, SeO₂R, SeO₂OR, SSR, SeSeR, SSeR, or SeSR;
Y₁, Y₂, Y₃, Y₄, Y₅, Y₆, Y₇, Y₈ and Y₉ are each independently selected from C, CR, CC(O)R, CC(O)OR, CC(O)N(R)R′, CN(R)R′, N, NR, NC(O)R, or NC(O)OR;
Z is R, O, N(R)R′, S, S(O), or SO₂;
R₃, R₄, R_{4′}, R₅, R_{5′}, R₆, R₇ and R_{7′} are each independently selected from —R, —OR, —N(R)R′, —C(O)R, —C(O)OR, —C(O)N(R)R′, a substituted or unsubstituted amino acid, C₁₋₁₂amino alcohol, or C₁₋₁₂carboxylic acid; and
R, R′ R″, R′″ and R″″ are each independently H or a substituted or unsubstituted C₁₋₁₀alkyl, C₂₋₁₀alkenyl, C₂₋₁₀alkynyl, C₆₋₁₀aryl, C₃₋₈cycloalkyl, C₄₋₆heterocyclyl or C₅₋₆heteroaryl.

8. The compound of claim 7, wherein the compound is a compound of Formula VI, and the carbon atom bound to R₃ is bound to an atom selected from the group consisting of Y₁, Y₂, Y₃, Y₄, Y₅, Y₆, Y₇, Y₈ and Y₉.

9. The compound of claim 7, wherein the compound is a compound of Formula VII, and the carbon atom bound to R₃ is bound to an atom selected from the group consisting of Y₁, Y₂, Y₃, Y₄, Y₅ and Y₆.

10. The compound of any preceding claim, wherein R₂ is selected from —C(R)═C(R)R′, —C≡CR, or wherein R₂ and X taken together form: 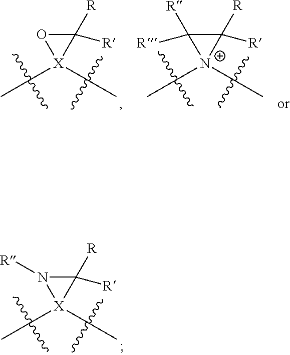
wherein R, R′ R″ and R′″ are each independently H or a substituted or unsubstituted C₁₋₁₀alkyl, C₂₋₁₀alkenyl, C₂₋₁₀alkynyl, C₆₋₁₀aryl, C₃₋₈cycloalkyl, C₄₋₆heterocyclyl or C₅₋₆heteroaryl.

11. The compound of any preceding claim, wherein the compound is not:

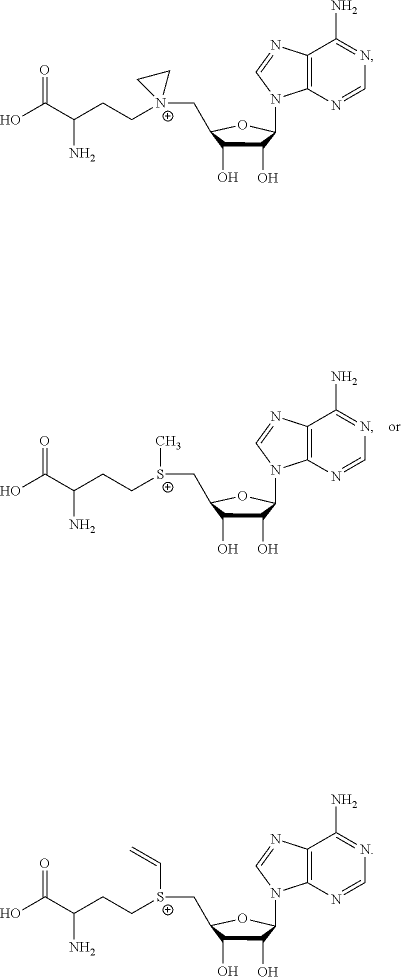

12. The compound of any preceding claim, wherein the compound is selected from the group consisting of: 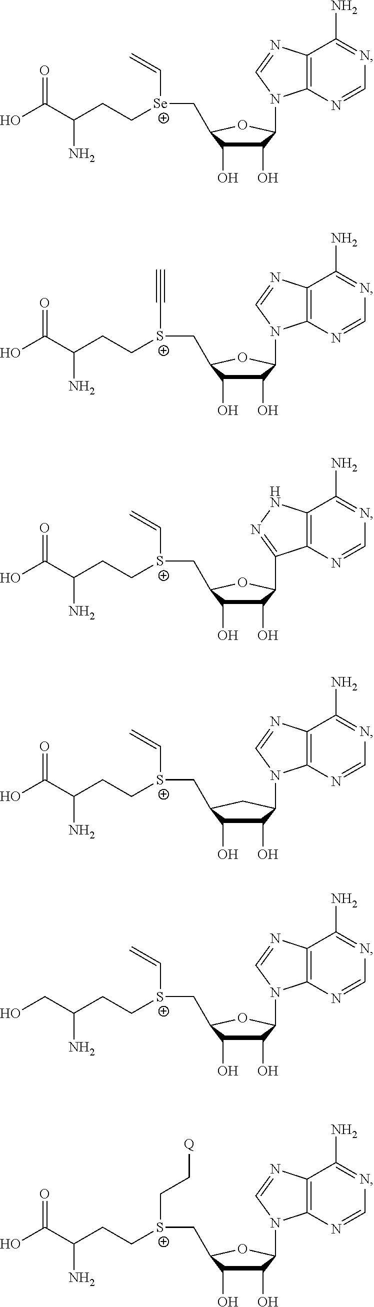 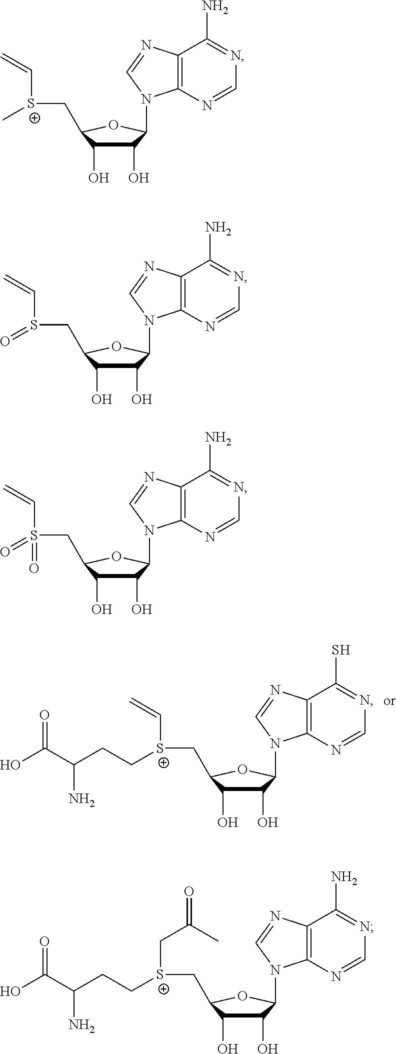
wherein Q is a halogen.

13. A compound of Formula IV: 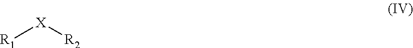
or a salt, hydrate or solvate thereof;
wherein
X is C, CR, N, NR, NOR, N(R)OR′, N(OR)OR′, P, PR, POR, P(R)R′, P(OR)R′, P(OR)OR′, P(O), P(R)(R′)R″, P(R)(R′)OR″, P(R)(OR′)OR″, P(OR)(OR′)OR″, P(O)R, P(O)OR, S, SR, SOR, S(R)R′, S(OR)R′, S(OR)OR′, S(O), S(R)(R′)R″, S(R)(R′)OR″, S(R)(OR′)OR″, S(OR)(OR′)OR″, S(O)R, S(O)OR, S(R)(R′)(R″)R′″, S(R)(R′)(R″)OR′″, S(R)(R′)(OR″)OR′″, S(R)(OR′)(OR″)OR′″, S(OR)(OR′)(OR″)OR′, S(O)(R)R′, S(O)(R)OR′, S(O)(OR)OR″, SO₂, Se, SeR, SeOR, Se(R)R′, Se(OR)R′, Se(OR)OR′, Se(O), Se(R)(R′)R″, Se(R)(R′)OR″, Se(R)(OR′)OR″, Se(OR)(OR′)OR″, Se(O)R, Se(O)OR, Se(R)(R′)(R″)R′″, Se(R)(R′)(R″)OR′″, Se(R)(R′)(OR″)OR′″, Se(R)(OR′)(OR″)OR′″, Se(OR)(OR′)(OR″)OR′, Se(RO)(R)R′, Se(RO)(R)OR′, Se(O)(OR)OR″, SeO₂, SS, SSe, SeS, or SeSe;
R₁ is —R, a substituted or unsubstituted amino acid, C₁₋₁₂amino alcohol, C₁₋₁₂carboxylic acid, —OR, ═O, or R₁ and X taken together are: 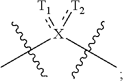
wherein T₁ and T₂ are each independently —OR or ═O;
R₂ is an electrophile;
wherein X and R₂ taken together can form a 3-to-10-membered ring; and
R, R′, R″ and R′″ are each independently H or a substituted or unsubstituted C₁₋₁₀alkyl, C₂₋₁₀alkenyl, C₂₋₁₀alkynyl, C₆₋₁₀aryl, C₃₋₈cycloalkyl, C₄₋₆heterocyclyl or C₅₋₆heteroaryl.

14. The compound of claim 13, wherein the compound is selected from:

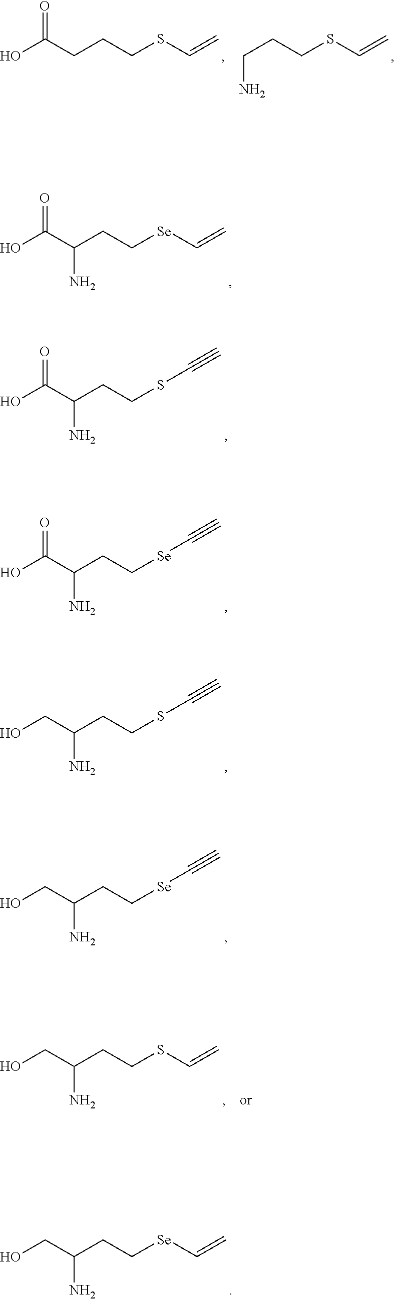

15. The compound of claim 13, wherein the compound is not:

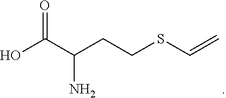

16. A method of making the compound of claim 1, comprising the steps of:
(a) providing a compound of Formula IV: 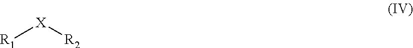
wherein
X is C, CR, N, NR, NOR, N(R)OR′, N(OR)OR′, P, PR, POR, P(R)R′, P(OR)R′, P(OR)OR′, P(O), P(R)(R′)R″, P(R)(R′)OR″, P(R)(OR′)OR″, P(OR)(OR′)OR″, P(O)R, P(O)OR, S, SR, SOR, S(R)R′, S(OR)R′, S(OR)OR′, S(O), S(R)(R′)R″, S(R)(R′)OR″, S(R)(OR′)OR″, S(OR)(OR′)OR″, S(O)R, S(O)OR, S(R)(R′)(R″)R′″, S(R)(R′)(R″)OR′″, S(R)(R′)(OR″)OR′″, S(R)(OR′)(OR″)OR′″, S(OR)(OR′)(OR″)OR′″, S(O)(R)R′, S(O)(R)OR′, S(O)(OR)OR″, SO₂, Se, SeR, SeOR, Se(R)R′, Se(OR)R′, Se(OR)OR′, Se(O), Se(R)(R′)R″, Se(R)(R′)OR″, Se(R)(OR′)OR″, Se(OR)(OR′)OR″, Se(O)R, Se(O)OR, Se(R)(R′)(R″)R′″, Se(R)(R′)(R″)OR′″, Se(R)(R′)(OR″)OR′″, Se(R)(OR′)(OR″)OR′″, Se(OR)(OR′)(OR″)OR′″, Se(RO)(R)R′, Se(RO)(R)OR′, Se(O)(OR)OR″, SeO₂, SS, SSe, SeS, or SeSe;
R₁ is —R, a substituted or unsubstituted amino acid, C₁₋₁₂amino alcohol, C₁₋₁₂carboxylic acid, —OR, ═O, or R₁ and X taken together are: 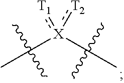
wherein T₁ and T₂ are each independently —OR or ═O;
R₂ is an electrophile;
wherein X and R₂ taken together can form a 3-to-10-membered ring; and
R, R′, R″ and R′″ are each independently H or a substituted or unsubstituted C₁₋₁₀alkyl, C₂₋₁₀alkenyl, C₂₋₁₀alkynyl, C₆₋₁₀aryl, C₃₋₈cycloalkyl, C₄₋₆heterocyclyl or C₅₋₆heteroaryl;
and
(b) contacting the compound of Formula IV with a nucleotide, nucleoside or derivative thereof.

17. The method of claim 16, wherein the compound of Formula IV is methionine and the nucleotide, nucleoside or derivative thereof is adenosine triphosphate.

18. The method of claim 16, wherein the compound of Formula IV is vinyl homocysteine or vinthionine and the nucleotide, nucleoside or derivative thereof is adenosine triphosphate or a derivative thereof.

19. The method of claim 16, wherein the reaction is catalyzed by methionine adenosyltransferase or a derivative thereof.

20. The method of claim 16, wherein the contacting step takes place in a cell.

21. A method of inhibiting a methyltransferase, the method comprising contacting the methyltransferase with a bisubstrate adduct comprising a methyltransferase substrate and a compound of claim 1, the compound of claim 1 comprising a compound of claim 13 and an adenosine triphosphate; wherein the bisubstrate adduct inhibits the methyltransferase.

22. The method of claim 21, wherein the methyltransferase substrate is a peptide comprising one or more isoaspartyl residues.

23. The method of claim 21, wherein the methyltransferase substrate is an amino acid, peptide, a protein, a DNA, an RNA, a carbohydrate, a lipid, a metabolite, a xenobiotic, a drug, or a small molecule.

24. The method of claim 21, wherein the compound is a compound of claim 12.

25. The method of claim 21, wherein the methyltransferase is in a cell.

26. The method of claim 21, wherein the compound of claim 13 and adenosine triphosphate are provided to a cell.

27. The method of claim 21, wherein the compound of claim 1 is generated from the compound of claim 13 and adenosine triphosphate in vivo.

28. The method of claim 21, wherein the bisubstrate adduct is formed by the methyltransferase in vivo.

29. A method of detecting a methyltransferase substrate, the method comprising:
(a) contacting a sample comprising the methyltransferase substrate with a compound of claim 1, the compound of claim 1 comprising a compound of claim 13 and adenosine triphosphate;
(b) generating a bisubstrate adduct comprising the methyltransferase substrate and the compound of claim 1; and
(c) detecting the methyltransferase substrate in the sample by detecting the bisubstrate adduct in the sample.

30. The method of claim 29, wherein the adenosine triphosphate is a formycin analog.

31. The method of claim 29, wherein the adenosine triphosphate is a fluorescent analog.

32. The method of claim 29, wherein the adenosine triphosphate is labeled with one or more of the group consisting of deuterium, tritium, ¹¹C, ¹²C, ¹³C, ¹⁴C, ¹⁶C, ¹⁷O, and ¹⁸O.

33. The method of claim 29, wherein the compound of claim 13 is labeled with one or more of the group consisting of fluorescent labels, deuterium, tritium, ¹¹C, ¹²C, ¹³C, ¹⁴C, ¹⁶O, ¹⁷O, ³²S, ³³S, ³⁴S, ³⁵S, ³⁵S, ⁷²Se, ⁷³Se, ⁷⁴Se, ⁷⁵Se, ⁷⁶Se, ⁷⁷Se, ⁷⁸Se, ⁷⁹Se, ⁸⁰Se, and ⁸²Se.

34. The method of claim 29, wherein the sample further comprises a methyltransferase.

35. The method of claim 34, wherein the methyltransferase generates the bisubstrate adduct.

36. The method of claim 29, wherein detecting the methyltransferase substrate comprises detecting a labeled bisubstrate adduct.

37. The method of claim 29 further comprising measuring the amount of methyltransferase substrate in the sample.

38. The method of claim 37, wherein the amount of methyltransferase substrate in the sample is determined by measuring the amount of labeled bisubstrate adduct in the sample.

39. The method of claim 35, wherein the amount of methyltransferase in the sample is determined by measuring the amount of bisubstrate adduct generated in the sample.

40. The method of claim 29, wherein the sample comprises a cell.

41. The method of claim 29, wherein contacting the sample with the compound of claim 1 comprises contacting the sample with the compound of claim 13 and the adenosine triphosphate.

42. The method of claim 41, wherein the compound of claim 1 is synthesized in the sample.

43. A method of isolating a methyltransferase, the method comprising:
(a) contacting a sample comprising the methyltransferase with a bisubstrate adduct comprising a methyltransferase substrate covalently linked to a compound of claim 1, the compound of claim 1 comprising a compound of claim 13 and adenosine triphosphate;
(b) incubating the sample with the bisubstrate adduct to allow binding of the substrate to methyltransferase;
(c) purifying the methyltransferase bound to the bisubstrate adduct from the sample; and
(d) isolating the methyltransferase from the bisubstrate adduct.

44. The method of claim 43, wherein the methyltransferase substrate is a peptide comprising one or more isoaspartyl residues.

45. The method of claim 43, wherein the methyltransferase substrate is an amino acid, peptide, a protein, a histone, a DNA, an RNA, a carbohydrate, a lipid, a metabolite, a xenobiotic, a drug, or a small molecule.

46. The method of claim 43, wherein purifying the methyltransferase comprises contacting the methyltransferase bound to the bisubstrate adduct to an antibody attached to a solid support.

47. The method of claim 46, wherein the antibody is specific for adenosine and its derivatives.

48. The method of claim 47, wherein the antibody is specific for adenosine triphosphate.

49. The method of claim 46, wherein the antibody is specific for the methyltransferase substrate.

50. The method of claim 43, wherein contacting the sample with the bisubstrate adduct comprises contacting the sample with the compound of claim 1 and the methyltransferase substrate to allow the methyltransferase to generate the bisubstrate adduct.

51. A method of isolating a methyltransferase substrate, the method comprising:
(a) contacting a sample comprising the methyltransferase substrate and a methyltransferase with a compound of claim 1, the compound of claim 1 comprising a compound of claim 13 and adenosine triphosphate;
(b) incubating the sample with the compound of claim 1 to allow the methyltransferase to form a bisubstrate adduct comprising the compound of claim 1 covalently linked to the methyltransferase substrate;
(c) purifying the methyltransferase bound to the bisubstrate adduct from the sample; and
(d) isolating the methyltransferase substrate by cleaving the covalent linkage between the methyltransferase substrate and the compound of claim 1.

52. The method of claim 51, wherein the methyltransferase substrate is a peptide comprising one or more isoaspartyl residues.

53. The method of claim 43, wherein the methyltransferase substrate is an amino acid, peptide, a protein (e.g., histone), a DNA, an RNA, a carbohydrate, a lipid, a metabolite, a xenobiotic, a drug, or a small molecule.

54. The method of claim 43, wherein purifying the methyltransferase comprises contacting the methyltransferase bound to the bisubstrate adduct to an antibody attached to a solid support.

55. The method of claim 54, wherein the antibody is specific for adenosine triphosphate.

56. The method of claim 54, wherein the antibody is specific for the methyltransferase substrate.

57. The method of any of claim 21, 29, 43, or 51, wherein the compound of claim 1 is synthesized in the sample.

58. The method of claim 21, wherein the methyltransferase substrate and the compound of claim 1 are covalently linked to form the bisubstrate adduct.

59. A method of treating a disease selected from Parkinson's disease, tropical parasitic diseases, and rheumatoid disease in a patient, the method comprising administering to a patient in need thereof a composition of any one of claims 1 to 15.

60. The method of claim 59, wherein the disease is caused by Leishmania promastigotes.

61. The method of claim 59, wherein the disease is selected from African sleeping sickness and highly tissue destructive disease.
